(19) United States
(12) Patent Application Publication
(10) Pub. No.: US 2011/0182881 A1
(43) Pub. Date: Jul. 28, 2011

(54) SIGNATURE AND DETERMINANTS ASSOCIATED WITH METASTASIS AND METHODS OF USE THEREOF

(75) Inventors: Lynda Chin, Brookline, MA (US); Kenneth L. Scott, Sugar Land, TX (US); Papia Ghosh, na, MA (US)

(73) Assignee: Dana-Farber Cancer Institute, Inc., Boston, MA (US)

(21) Appl. No.: 13/001,203

(22) PCT Filed: Jun. 26, 2009
(86) PCT No.: PCT/US09/48862

§ 371 Date: Mar. 31, 2011

Related U.S. Application Data
(60) Provisional application No. 61/075,933, filed on Jun. 26, 2008.

(51) Int. Cl. A61K 39/395; G01N 33/53; C40B 40/00; A61K 38/20; A61P 35/00; G01N 27/447

(52) U.S. Cl. 424/130.1; 436/501; 435/7.92; 435/7.1; 506/13; 435/7.2; 514/19.3; 204/450

(57) ABSTRACT

The present invention provides methods of detecting cancer using biomarkers.

### RELATED APPLICATION

This application claims the benefit of U.S. Ser. No. 61/075,933, filed Jun. 26, 2008 the contents of which is incorporated herein by reference in its entirety.

### FIELD OF THE INVENTION

The present invention relates generally to the identification of biological signatures associated with and genetic determinants effecting cancer metastasis and methods of using such biological signatures and determinants in the screening, prevention, diagnosis, therapy, monitoring, and prognosis of cancer.

### BACKGROUND OF THE INVENTION

Metastasis is the cardinal feature of most lethal solid tumors and represents a complex multi-step biological process driven by an ensemble of genetic or epigenetic alterations that confer a tumor cell the ability to bypass local control and invade through surrounding matrix, survive transit in vasculature or lymphatics, ultimately colonize on foreign soil and grow (Gaorav P. Gupta and Joan Massagué (2006) Cell). It is the general consensus that such metastasis-conferring genetic events can be acquired stochastically as tumor grows and expands; indeed, total tumor burden is a positive predictor of metastatic risk. On the other hand, mounting evidence has promoted the thesis that some tumors may be endowed (or not) from the earliest stages with the capacity to metastasize. That some tumors are “hard-wired” for metastasis early in their life history is supported by clinical observation of widely varying outcomes among tumors of the equivalent early stage (i.e., similar tumor burden). Correspondingly, it has been shown that transcriptomic state of a metastasis more similar to its matched primary than to other metastasis (Perou et al, 2000). In addition, it has been demonstrated that wholesale genomic aberrations in a cancer genome occurs early at the transition from benign to malignant stage (Chin, K., de Solorzano, C. O., Knowles, D., Jones, A., Chou, W., Rodriguez, E. G., Kuo, W. L., Ljung, B. M., Chew, K., Myambo, K., et al. (2004). In situ analyses of genome instability in breast cancer. Nat Genet 36, 984-988.; Rudolph, K. L., Millard, M., Bosenberg, M. W., and DePinho, R. A. (2001). Telomere dysfunction and evolution of intestinal carcinoma in mice and humans. Nat Genet 28, 155-159. Other authors include Marcus Bosenberg, suggesting that the particular complement of genetic events acquired at that early stage of evolution will ultimately dictate, at least in part, the biological behavior of tumor, including its metastatic potential. Thus, we posit that the genetic determinants of a tumor's metastatic potential are pre-existing in early stage primary malignancies, and such determinants are functionally active in the very processes responsible for metastatic dissemination. Therefore, such metastasis determinants are not only potential therapeutic targets but also determinants of aggressiveness of the cancerous disease, hence the metastatic determinents are also prognosic determinants.

### SUMMARY OF THE INVENTION

The present invention relates in part to the discovery that certain biological markers (referred to herein as “DETERMINANTS”), such as proteins, nucleic acids, polymorphisms, metabolites, and other analytes, as well as certain physiological conditions and states, are present or altered in subjects with an increased risk of developing a metastatic tumor.

Accordingly in one aspect the invention provides a method with a for assessing a risk of development of a metastatic tumor in a subject. Risk of developing a metastatic tumor is determined by measuring the level of an effective amount of a DETERMINANT in a sample from the subject. An increased risk of developing a metastatic tumor in the subject is determined by measuring a clinically significant alteration in the level of the DETERMINANT in the sample. Alternatively, an increased risk of developing a metastatic tumor in the subject is determined by comparing the level of the effective amount DETERMINANT to a reference value. In some aspects the reference value is an index.

In another aspect the invention provides a method for assessing the progression of a tumor in a subject by detecting the level of an effective amount a DETERMINANTS in a first sample from the subject at a first period of time, detecting the level of an effective amount of DETERMINANTS in a second sample from the subject at a second period of time and comparing the level of the DETERMINANTS detected in to a reference value. In some aspects the first sample is taken from the subject prior to being treated for the tumor and the second sample is taken from the subject after being treated for the tumor.

In a further aspect the invention provides a method for monitoring the effectiveness of treatment or selecting a treatment regimen for a metastatic tumor by detecting the level of an effective amount of DETERMINANTS in a first sample from the subject at a first period of time and optionally detecting the level of an effective amount of DETERMINANTS in a second sample from the subject at a second period of time. The level of the effective amount of DETERMINANTS detected at the first period of time is compared to the level detected at the second period of time or alternatively a reference value. Effectiveness of treatment is monitored by a change in the level of the effective amount of DETERMINANTS from the subject.

In yet another aspect the invention provides a method of treating a patient with a tumor, by identifying a patient with a tumor where an effective amount of DETERMINANTS are altered in a clinically significant manner as measured in a sample from the tumor, an treating the patient with a therapeutic regimen that prevents or reduces tumor metastasis.

In one aspect the invention provide a method of selecting a tumor patient in need of adjuvant treatment by assessing the risk of metastasis in the patient by measuring an effective amount of DETERMINANTS where a clinically significant alteration two or more DETERMINANTS in a tumor sample from the patient indicates that the patient is in need of adjuvant treatment.

In a further aspect the invention provides a method of informing a treatment decision for a tumor patient by obtaining information on an effective amount of DETERMINANTS in a tumor sample from the patient, and selecting a treatment regimen that prevents or reduces tumor metastasis in the patient if two or more DETERMINANTS are altered in a clinically significant manner.

In various embodiments the assessment/monitoring is achieved with a predetermined level of predictability. By predetermined level of predictability is meant that that the method provides an acceptable level of clininal or diagnostic accuracy. Clinical and diagnositic accuracy ais determined by methods known in the art, such as by the methods described herein.

A DETERMINANT includes for example DETERMINANT 1-360 described herein. One, two, three, four, five, ten or more DETERMINANTS are measured. Preferably, at least two DETERMINANTS selected from DETERMINANTS 1-25, 41, 61, 62, 63, 66, 74, 96, 99, 103, 126, 135, 137, 138, 177, 190, 210, 212, 217, 218, 227, 239, 261, and 271 are measured. Optionally, the methods of the invention further include measuring at least one standard parameters associated with a tumor.

The level of a DETERMINANT is measured electrophoretically or immunochemically. For example the level of the determinant is detected by radioimmunoassay, immunofluorescence assay or by an enzyme-linked immunosorbent assay.

The subject has a primary tumor, a recurrent tumor, or a metastatic tumor. In some aspects the sample is taken for a subject that has previously been treated for the tumor. Alternatively, the sample is taken from the subject prior to being treated for the tumor. The sample is a tumor biopsy such as a core biopsy, an excisional tissue biopsy or an incisional tissue biopsy, or a blood sample with circulating tumor cells.

Also included in the invention is a metastatic tumor reference expression profile containing a pattern of marker levels of an effective amount of two or more markers selected from DETERMINANTS 1-360. Preferably, the profile contains a pattern of marker levels of DETERMINANTS 1-25, 41, 61, 62, 63, 66, 74, 96, 99, 103, 126, 135, 137, 138, 177, 190, 210, 212, 217, 218, 227, 239, 261, and 271. Also included is a machine readable media containing one or more metastatic tumor reference expression profiles and optionally, additional test results and subject information. In another aspect the invention provides a kit comprising a plurality of DETERMINANT detection reagents that detect the corresponding DETERMINANTS. The detection reagent is for example antibodies or fragments thereof, oligonucleotides or aptamers.

In a further aspect the invention provides a DETERMINANT panel containing one or more DETERMINANTS that are indicative of a physiological or biochemical pathway associated metastasis or the progression of a tumor. The physiological or biochemical pathway includes for example,

In yet another aspect, the invention provides a way of identifying a biomarker that is prognostic for a disease by identifying one or more genes that are differentially expressed in the disease compared to a control to produce a gene target list; and identifying one or more genes on the target list that is associated with a functional aspect of the progression of the disease. The functional aspect is for example, cell migration, angiogenesis, extracellular matrix degradation or anoikis resistance. Optionally, the method includes identifying one or more genes on the gene target list that comprise an evolutionarily conserved change to produce a second gene target list. The disease is for example cancer such as metastatic cancer.

Compounds that modulates the activity or expression of a DETERMINANT are identified by providing a cell expressing the DETERMINANT, contacting (e.g., in vivo, ex vivo or in vitro) the cell with a composition comprising a candidate compound; and determining whether the substance alters the expression of activity of the DETERMINANT. If the alteration observed in the presence of the compound is not observed when the cell is contacted with a composition devoid of the compound, the compound identified modulates the activity or expression of a DETERMINANT.

Cancer is treated in a subject be administering to the subject a compound that modulates the activity or expression of a DETERMINANT or by administering to the subject an agent that modulates the activity or expression of a compound that is modulated by a DETERMINANT. The compound can be, e.g., (i) a DETERMINANT polypeptide; (ii) a nucleic acid encoding a DETERMINANT (iii) a nucleic acid that decreases the expression or activity of a nucleic acid that encodes DETERMINANT such as, and derivatives, fragments, analogs and homologs thereof (iv a polypeptide that decreases the expression or activity if a DETERMINANT such as an antibody specific for the DETERMINANT. The term “antibody” (Ab) as used herein includes monoclonal antibodies, polyclonal antibodies, multispecific antibodies (e.g., bispecific antibodies), humanized or human antibodies, Fv antibodies, diabodies and antibody fragments, so long as they exhibit the desired biological activity. For example the compound is TGFβ and the agent is a TGFβ inhibitor. Another example is CXCR4 and the agent is a CXCR4 antagonist.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1 shows Melanocyte-specific MET expression promotes formation of cutaneous melanoma. (A) Melanocytes were harvested from the indicated animals and adapted to culture. Total RNA was extracted from cultured melanocytes grown in the presence or absence of doxycycline (DOX), and expression of MET (Tg MET) was assayed by RT-PCR using transgene-specific primers. R15, ribosomal protein R15 internal control; −RT, no reverse transcriptase PCR control (B) Primary tumors (T1-T6) were harvested from iMet animals on doxycycline and assessed for expression of the melanocytic markers Tyrosinase, TRP1 and Dct by RT-PCR using gene-specific primers. XB2, mouse keratinocyte cell line; B16F10, mouse melanoma cell line; R15, ribosomal protein R15 internal control; −RT, no reverse transcriptase PCR control. (C) Melanocyte-specific immunohistochemical staining of S100 in a MET-induced primary melanoma. t, tumor; f, folicule; fm, folicular melanocytes; a, adipocytes. (D) Immunohistochemical staining of total c-Met and phosphorylated c-Met in a MET-induced primary melanoma. (E) RT-qPCR was performed to analyze HGF expression in MET-induced primary melanomas (T1-T6). Tumor expression data is normalized to expression in two Ink4a/Arf^{-/-} melanocyte cell lines.

FIG. 2. shows Met activation drives development of metastatic melanomas and promotes lung seeding. (A). Boyden chambers were seeded with 5×10⁴ iMet tumor cells (line BC014) in serum-free media. Chambers were placed in chemo-attractant (media containing 10% serum) without and with 50 ng/ml recombinant HGF and incubated for 24 hrs. Invasive cells were visualized by staining with crystal violet. (B) H&E stained sections of a primary cutaneous spindle cell melanoma in the dorsal skin of an iMet transgenic mouse induced with doxycycline and distal metastases residing in lymph node adrenal gland and lung. (C) 5×10⁵ cells were injected in the tail vein of SCID and mice followed for formation of lung nodules, a correlate of metastatic seeding. Left panel: H&E stained section of nodule-free lung tissue harvested from SCID animals tail vein injected with an HRAS* melanoma cell line ( 0/4 mice); Right panel: H&E stained section of nodule-infiltrated lung tissue harvested from SCIDs tail vein-injected with the MET-driven BC014 cell line (iMet) (¾ mice). t, tumor.

FIG. 3. shows multi-dimensional cross species genomic analyses coupled with a low-complexity functional genetic screen for cell invasion identifies metastasis determinants (A) Differentially expressed genes (1597 probe sets) by SAM analyses of expression profiles generated from iHRAS* and iMet cutaneous melanomas were intersected by ortholog mapping with genes resident within regions of amplifications and deletions in human metastatic melanoma, or with differentially expressed genes between human primary and metastatic melanoma to define 360 candidates. (B) Ingenuity Pathway Analysis of differentially expressed genes between iHRAS* and iMet mouse melanomas (1597 probe sets, top) and cross-species integrated gene list (360 filtered gene list, bottom) were compared to 9 randomly drawn gene sets of equal size. Top 4 significant functional classifications are shown. Dashed lines represent significance by IPA. (C) Flowchart depicting the low-complexity genetic screen for invasion. 230 clones representing 199 of the 295 up-regulated/amplified candidates expressed in a lentiviral system were individually transduced into TERT-immortalized human primary melanocytes (HMEL468) and assayed for invasion in a 96-well matrigel invasion plate. Invasiveness was measured via florescence-mediated quantitation and values were normalized to GFP controls. Candidates scoring greater than 2× standard deviations away from vector control in two independent screens (n=45) were selected for secondary validation screen in HMEL468 or in WM3211 using standard 24-well matrigel invasion chambers. (D) Histogram summary of the low-complexity genetic screen for pro-invasion genes. HMEL468 primed melanocytes were transduced with individual pro-metastasis candidate cDNA virus, followed by loading onto 96-well transwell invasion assay plates. Invasiveness was measured via florescence-mediated quantitation and values were normalized to empty vector control. Candidate cDNAs driving invasion 2× standard deviations from the GFP controls in two independent screening efforts were considered primary screen hits (n=45). (E) Summary histogram of fold-increase in invasive activity relative to control 31 validated metastasis determinants.

FIG. 4 shows Automated Quantitative Analysis (AQUA®) of protein expression for representative determinants (A) Fascin1 (FSCN1) and (B) HSF1 performed on tissue micrraorrays (TMA) of nevi, primary and metastatic melanoma tumor specimens as described [Camp, R. L., Chung, G. G., & Rimm, D. L., Automated subcellular localization and quantification of protein expression in tissue microarrays. Nat Med 8 (11), 1323-1327 (2002)]. Informative cores were assessed for AQUA® scores for FSCN1 and HSF1 staining in the cytoplasmic and nuclear cellular compartments, respectively. Significance (S; 5%) based on Fisher's test. See Table 2 for results summary

FIG. 5 shows (A) K-means hierarchal clustering and (B) Kaplan-Meier analysis for overall (top) and metastasis-free (bottom) survivals of two subclasses from above in a cohort of 295 Stage I-II breast cancers [breast cancer data from: van de Vijver, M. J. et al., A gene-expression signature as a predictor of survival in breast cancer. N Engl J Med 347 (25), 1999-2009 (2002); van't Veer, L. J. et al., Gene expression profiling predicts clinical outcome of breast cancer. Nature 415 (6871), 530-536 (2002)]

FIG. 6 show the in vitro anoikis screen methodology. (A) In vitro anoikis screen strategy. (B) Rat intestinal epithelial (RIE) cells have reduced viability when plated on low-attachment plates. RIE cells were plated on either 96-well ULC plates or adherent plates for 24 hrs. ATP levels were measured for cell viability and given as a ratio of level at time 24 hr/0 hr. (C) RIE express V5-mTrkB. RIE cells were infected and at 48 hrs cell lysate was isolated and resolved by SDS-PAGE. Western blot analysis was done with α-V5 antibody.

FIG. 7 shows various genes confer anoikis resistance to RIE cells. RIE cells were infected with retrovirus expressing one of the candidate genes, plated on ultra-low cluster plates and viability of cells was measured 24 hrs post-plating. Values are given relative to 0 hr viability. All readings were done in triplicate. Highlightedare readings of empty vector, BDNF or mTrkB (positive controls).

FIG. 8 shows the twenty candidate genes that conferred anoikis resistance to Rat Intestinal Epitheal (RIE) cells greater than two standard deviations from the median. Nine candidate genes (HNRPR, CDC20, PRIM2A, HRSP12, ENY2, MGC14141, RECQL, STK3, and MX2) gave greater than 1 standard deviation from the median on two independent screens. These genes are located on the indicated chromosomes.

FIG. 9 Genes confer ability of RIE cells to attach after maintenance in suspension. RIE cells expressing a candidate gene were plated on ULC plates for 24 hrs. Cells in suspension were transferred to adherent plates and 24 hrs hours later attached cells were stained with crystal violet. Cell viability is given as 24 hr/0 hr. All readings were done in triplicate.

FIG. 10 shows metastasis determinants promote tumorigenicity (A) HMEL468 cells stably expressing either GFP or the indicated metastasis determinants were injected subcutaneously into SCID mice (n=6), which were monitored for tumor formation by clinical exam. Shown are representative H&E stained primary tumors (t) exhibiting local invasion through surrounding muscle fiber (m) and adipocytes (a). (B) Table summarizing data collected form determinant-driven tumorigenesis assays.

FIG. 11 illustrates that determinant HOXA1 promotes cell invasion and lung seeding capacity. (A) Ectopic expression of HOXA1 in HMEL468 led to increased activation of FAK (Tyr397; left panel) and corresponding increase in invasion through matrigel in transwell invasion assays (right panel; quantitated in FIG. 11C) (B) Western blot analysis for HOXA1-V5 to confirm HOXA1 expression in WM115 and WM3211 transduced cell lines (left panel) and representative images of the transwell invasion assays (right panel) quantitated in FIG. 11C. (C) Quantitation of invasion chamber data presented in FIG. 11A-B. (D) HMEL468 cells stably expressing either GFP or HOXA1 were injected intravenously into the tail vein of SCID mice (n=6) and examined on necropsy for lung nodules at 12 weeks post injection. Macroscopic (and microscopic) lung nodules were detected in 50% of HOXA1 cohort (n=3) but in none of the control. Representative H&E photomicrographs of lung nodule (t) and surrounding lung parenchyma (1) excised from one HMEL468-HOXA1-injected animal. (E)WM115 melanoma cells expressing either empty vector (EV) or HOXA1 were injected subcutaneously into nude mice and measured 46 days post-injection. (F) Representative metastases of the lung isolated from a nude mouse bearing intradermally-injected WM115-HOXA1 cells.

FIG. 12 shows HOXA1-driven transcriptome analysis identified a Smad3 network defined by Ingenuity Pathway Analysis. A molecule network generated using Ingenuity Pathways Analysis (Ingenuity Systems Inc.). The network is displayed graphically as nodes (genes) and edges (the biological relationships between nodes). Solid lines represent direct interactions and dashed lines represent indirect interactions. Red and green colors denote genes that were over-expressed or under-expressed in the transcriptome analysis, respectively. The shapes of the objects represent the functional families to which the proteins belong. Refer Supplementary table s3 for gene family and descriptions. (B). The indicated HOXA1-transduced cell lines were assessed for SMAD3 expression using RT-qPCR. Values were calculated relative to GAPDH internal control and GFP experimental control. Error bars represent standard error.

FIG. 13 shows ectopic expression of HOXA1 enhances cell invasion through up-regulation of the TGFβ signaling response. (A)WM115 cells ectopically expressing HOXA1 were transfected with the TGFβ-inducible 3TP-Lux luciferase reporter, followed by treatment with or without TGFβ to assess responsiveness compared to the GFP-expressing control. Error bars represent standard error; Two-tailed t-test: −TGFβ p=0.003; +TGFβ, p<0.0001. (B). Whole-cell lysates from GFP or HOXA1 stably expressing WM115 cells propagated in either 10% serum or 1% serum with or without TGFβ were analyzed by Western blot using the indicated antibodies. (C). WM115 cells stably expressing HOXA1 were transduced with either SMAD3 shRNA (shSMAD3) or non-targeting shRNA (shNT) and loaded onto matrigel transwell invasion chambers to assay cell invasion in comparison to the WM115 parental cell line transduced with GFP control virus (GFP). Representative images of invasion chambers are shown in right panels. Two-tailed t-test: GFP vs. shNT, p=0.0008; shNT vs. shSMAD3, p=0.0022. (D) WM115 melanoma cells expressing either empty vector (EV) or HOXA1 were injected subcutaneously into nude mice. Resulting xenograft tumor sections were immunostained with anti-phospho-SMAD3 to confirm SMAD3 activation in HOXA1 tumor specimens.

FIG. 14. Ink4a/Arf^{-/-} mouse-derived melanocytes transduced with HRAS* (M3HRAS) over-expressing FSCN1 or HOXA1 exhibit (A) enhanced invasion through matrigel in transwell invasion assays (B) enhanced subcutaneous tumor growth in nude mice and (C) increased lung nodule formation following intravenous tail vein injection into SCID mice. Note that in C, the lung/body mass index difference for the FSCN1 cohort is not significant due to the relative good health of those animals at the assay endpoint that was mandated by the extremely ill HOXA1 cohort.

FIG. 15 RNA extracted from (A) WM115 melanoma cells and (B) transformed human melanocytes (HMEL468) expressing either empty vector (control group) or HOXA1 (Group 1) was used for quantitative qPCR analysis using RT² Profiler PCR Arrays (Supperarray) to analyze expression of a panel of genes associated with metastasis. Resulting xenograft tumor sections were immunostained with anti-CXCR4 to confirm over-expression in HOXA1 tumor specimens (FIG. 13). Shown are genes meeting threshold differential expression between control and experimental groups.

FIG. 16 WM115 melanoma cells and transformed human melanocytes (HMEL468) expressing either empty vector (EV) or HOXA1 were injected subcutaneously into nude mice. Resulting xenograft tumor sections were immunostained with anti-CXCR4 to confirm over-expression in HOXA1 tumor specimens.

FIG. 17 (A) QuantiGene analysis of RNA expression. of UBE2C in a cohort of Spitz nevi and melanoma FFPE specimen. (B) Primary Ink4a/Arf-deficient MEFs were transfected with the indicated vectors expressing HRASV12, MYC and UBE2C. Vec=LacZ vector control; bars indicate±S.D.

FIG. 18 (A) WM3211 melanoma cells stably-expressing empty vector (ev), Geminin or Nedd9 (positive control) were assayed for invasion through matrigel in transwell invasion assays. (B) Immunoblot analysis of total cell lysates extracted from WM3211 cells stably-expressing empty vector (ev), Geminin or Caveolin1 (negative control). Anti-phospho FAK and anti-phospho ERK represent activated FAK and ERK species, respectively. (C) WM3211 cells stably-expressing empty vector (EV) or Geminin (GEMN) were immunostained for phospho-FAK (P-FAK; red) to confirm increased FAK activation observed in FIG. 18B.

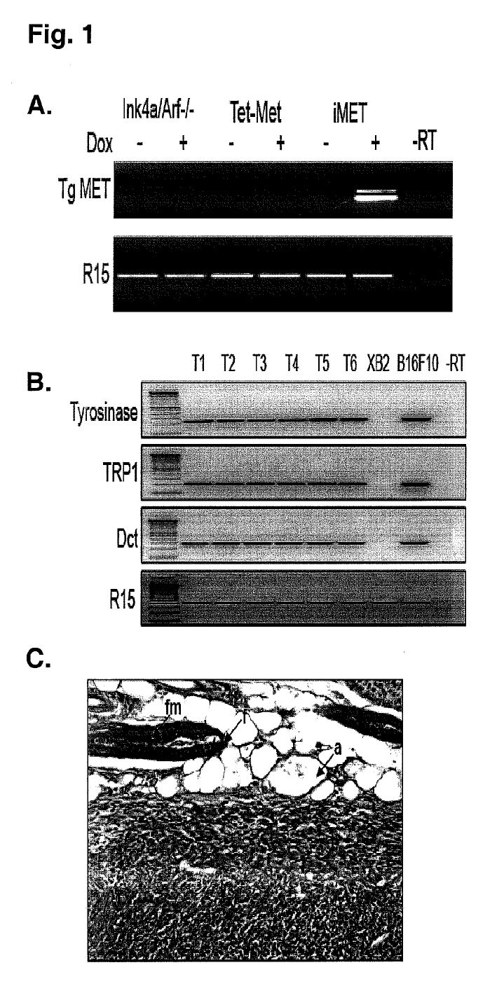

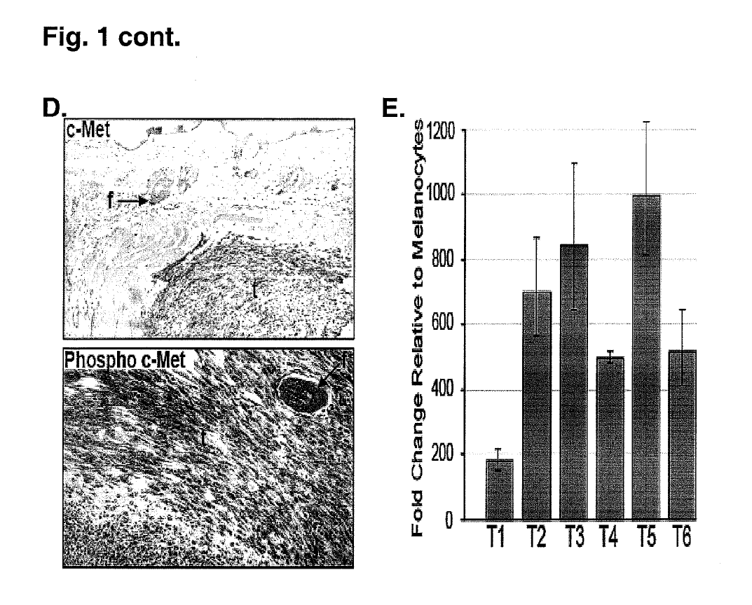

### DETAILED DESCRIPTION OF THE INVENTION

The present invention relates to the identification of signatures associated with and determinants conferring subjects with a metastatic tumor or are at risk for developing a metastatic tumor.

Cross-species comparison between human and mouse datasets has proven to provide a biological filter for the identification of causal cancer genes relevant to human melanoma biology. In the present study, two mouse models of melanomas whose primary tumors exhibit distinct potential to metastasize were used to identify genes that were differentially expressed in metastasis. An expression profile comparison of primary tumors originating from metastatic (iMet) and non-metastatic (iHRAS*) GEM models identified a list of 1597 differentially expressed genes that were prioritized by biological filtering by cross-species analysis and overlap with patterns of amplification and deletion obtained by array-CGH. It was hypothesized that evolutionarily conserved changes (e.g. in mouse and human) are more likely to be essential; therefore, triangulation of expression data from the GEM models (with advantages of defined genetic backgrounds and clear phenotype correlation) with genomic data from huma metastatic tumors allowed for prioritization and assigned human relevance to the 1597 candidates.

A phenotype-driven evolutionarily-conserved metastasis candidates list of 295 upregulated/amplified and 65 downregulated/deleted genes were identified by comparing the transcriptomes of two genetically engineered mouse models of cutaneous melanomas with differential metastatic potential, followed by triangulating with genomic and transcriptomic profiles of human primary and metastatic melanomas. These candidates were enlisted into low-complexity genetic screens for invasion, anoikis resistance or survival in circulation and colonization, corresponding to three major steps in metastatic spread (i.e. escaping the primary tumor site, circulation, lastly colonize and proliferate at distal foreign site. Thus far, the invasion screen has defined thirty-one (31) validated metastasis determinants capable of conferring pro-invasion activity to TERT-immortalized human melanocyte and melanoma cells. It is expected that independent subsets of the metastasis candidates will be defined as additional determinants from anoikis resistance or colonization screens that are only partially, if at all, overlapping with determinants from the invasion screen. Thus far, the anoikis resistant screen has defined nine (9) validated determinants capable of conferring survival in suspension, without overlap with the invasion determinants. These determinants together or a subset of will cover major steps involved in metastatic dissemination.

It is recognized that primary tumors are genetically heterogeneous. If metastasis determinants in a sub-population within a primary tumor confers it a proliferative advantage and ultimately drive its dissemination to distal sites, it is then expected that the metastatic derivative will be more homogeneous relative to its primary counterpart and therefore manifesting a progression-correlated pattern of expression for such metastasis determinants. To assess the expression pattern of 25 of these determinants, we took advantage of the compendium of expression profiling data on Oncomine (Rhodes D. R. et al. ONCOMINE: a cancer microarray database and integrated data-mining platform. Neoplasia 6, 1-6. 2004). However, although the majority of these 25 metastasis determinants have not been specifically implicated in invasion or metastasis, every one of them exhibit an expression pattern significantly correlated with advancing tumor grade or prognosis in both melanoma and non-melanoma solid tumors. For examples, 12 of the 25 determinants show increased expression in metastasis relative primary disease. In brain (gliomas) tumors, another mesenchymal tumor like melanoma, 13 of the metastasis determinants exhibited progression correlated expression pattern, namely, increasing expression in higher grade gliomas. Of these, six showed positive correlation with outcome. In prostate adenocarcinoma, ten of the metastasis determinants exhibited significant increase in expression from primary to metastasis. In lung, five exhibited correlation with increasing tumor grades. The most significant overlap was observed with breast adenocarcinoma, where 13 of the 25 metastasis determinants showed correlation with stages or grades of tumor progression; moreover, 13 of the determinants were reported to be correlated with prognosis.

Accordingly, the invention provides methods for identifying subjects who have a metastatic tumor, or who at risk for experiencing a metastatic tumor by the detection of determinants associated with the metatstatic tumor, including those subjects who are asymptomatic for the metastatic tumor. These signatures and determinants are also useful for monitoring subjects undergoing treatments and therapies for cancer, and for selecting or modifying therapies and treatments that would be efficacious in subjects having cancer, wherein selection and use of such treatments and therapies slow the progression of the tumor, or substantially delay or prevent its onset, or reduce or prevent the incidence of tumor metastasis.

### DEFINITIONS

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, specificity, positive predictive values (PPV) or negative predictive values (NPV), or as a likelihood, odds ratio, among other measures.

“Determinant” in the context of the present invention encompasses, without limitation, proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures. Determinants can also include mutated proteins or mutated nucleic acids. Determinants also encompass non-blood borne factors or non-analyte physiological markers of health status, such as “clinical parameters” defined herein, as well as “traditional laboratory risk factors”, also defined herein. Determinants also include any calculated indices created mathematically or combinations of any one or more of the foregoing measurements, including temporal trends and differences. Where available, and unless otherwise described herein, determinants which are gene products are identified based on the official letter abbreviation or gene symbol assigned by the international Human Genome Organization Naming Committee (HGNC) and listed at the date of this filing at the US National Center for Biotechnology Information (NCBI) web site (http://www.ncbi.nlm.nih.gov/sites/entrez?db=gene), also known as Entrez Gene.

“DETERMINANT” OR “DETERMINANTS” encompass one or more of all nucleic acids or polypeptides whose levels are changed in subjects who have a metastatic tumor or are predisposed to developing a metastatic tumor, or at risk of a metastatic tumor. Individual DETERMINANTS are summarized in Table 1 and are collectively referred to herein as, inter alia, “metastatic tumor-associated proteins”, “DETERMINANT polypeptides”, or “DETERMINANT proteins”. The corresponding nucleic acids encoding the polypeptides are referred to as “metastatic tumor-associated nucleic acids”, “metastatic tumor-associated genes”, “DETERMINANT nucleic acids”, or “DETERMINANT genes”. Unless indicated otherwise, “DETERMINANT”, “metastatic tumor-associated proteins”, “metastatic tumor-associated nucleic acids” are meant to refer to any of the sequences disclosed herein. The corresponding metabolites of the DETERMINANT proteins or nucleic acids can also be measured, as well as any of the aforementioned traditional risk marker metabolites.

Physiological markers of health status (e.g., such as age, family history, and other measurements commonly used as traditional risk factors) are referred to as “DETERMINANT physiology”. Calculated indices created from mathematically combining measurements of one or more, preferably two or more of the aforementioned classes of DETERMINANTS are referred to as “DETERMINANT indices”.

“Clinical parameters” encompasses all non-sample or non-analyte biomarkers of subject health status or other characteristics, such as, without limitation, age (Age), ethnicity (RACE), gender (Sex), or family history (FamHX).

“Circulating endothelial cell” (“CEC”) is an endothelial cell from the inner wall of blood vessels which sheds into the bloodstream under certain circumstances, including inflammation, and contributes to the formation of new vasculature associated with cancer pathogenesis. CECs may be useful as a marker of tumor progression and/or response to antiangiogenic therapy.

“Circulating tumor cell” (“CTC”) is a tumor cell of epithelial origin which is shed from the primary tumor upon metastasis, and enters the circulation. The number of circulating tumor cells in peripheral blood is associated with prognosis in patients with metastatic cancer. These cells can be separated and quantified using immunologic methods that detect epithelial cells.

“FN” is false negative, which for a disease state test means classifying a disease subject incorrectly as non-disease or normal.

“FP” is false positive, which for a disease state test means classifying a normal subject incorrectly as having disease.

A “formula,” “algorithm,” or “model” is any mathematical equation, algorithmic, analytical or programmed process, or statistical technique that takes one or more continuous or categorical inputs (herein called “parameters”) and calculates an output value, sometimes referred to as an “index” or “index value.” Non-limiting examples of “formulas” include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations. Of particular use in combining DETERMINANTS and other determinant are linear and non-linear equations and statistical classification analyses to determine the relationship between levels of DETERMINANTS detected in a subject sample and the subject's risk of metastatic disease. In panel and combination construction, of particular interest are structural and synactic statistical classification algorithms, and methods of risk index construction, utilizing pattern recognition features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, among others. Other techniques may be used in survival and time to event hazard analysis, including Cox, Weibull, Kaplan-Meier and Greenwood models well known to those of skill in the art. Many of these techniques are useful either combined with a DETERMINANT selection technique, such as forward selection, backwards selection, or stepwise selection, complete enumeration of all potential panels of a given size, genetic algorithms, or they may themselves include biomarker selection methodologies in their own technique. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Bootstrap, Leave-One-Out (LOO) and 10-Fold cross-validation (10-Fold CV). At various steps, false discovery rates may be estimated by value permutation according to techniques known in the art. A “health economic utility function” is a formula that is derived from a combination of the expected probability of a range of clinical outcomes in an idealized applicable patient population, both before and after the introduction of a diagnostic or therapeutic intervention into the standard of care. It encompasses estimates of the accuracy, effectiveness and performance characteristics of such intervention, and a cost and/or value measurement (a utility) associated with each outcome, which may be derived from actual health system costs of care (services, supplies, devices and drugs, etc.) and/or as an estimated acceptable value per quality adjusted life year (QALY) resulting in each outcome. The sum, across all predicted outcomes, of the product of the predicted population size for an outcome multiplied by the respective outcome's expected utility is the total health economic utility of a given standard of care. The difference between (i) the total health economic utility calculated for the standard of care with the intervention versus (ii) the total health economic utility for the standard of care without the intervention results in an overall measure of the health economic cost or value of the intervention. This may itself be divided amongst the entire patient group being analyzed (or solely amongst the intervention group) to arrive at a cost per unit intervention, and to guide such decisions as market positioning, pricing, and assumptions of health system acceptance. Such health economic utility functions are commonly used to compare the cost-effectiveness of the intervention, but may also be transformed to estimate the acceptable value per QALY the health care system is willing to pay, or the acceptable cost-effective clinical performance characteristics required of a new intervention.

For diagnostic (or prognostic) interventions of the invention, as each outcome (which in a disease classifying diagnostic test may be a TP, FP, TN, or FN) bears a different cost, a health economic utility function may preferentially favor sensitivity over specificity, or PPV over NPV based on the clinical situation and individual outcome costs and value, and thus provides another measure of health economic performance and value which may be different from more direct clinical or analytical performance measures. These different measurements and relative trade-offs generally will converge only in the case of a perfect test, with zero error rate (a.k.a., zero predicted subject outcome misclassifications or FP and FN), which all performance measures will favor over imperfection, but to differing degrees.

“Measuring” or “measurement,” or alternatively “detecting” or “detection,” means assessing the presence, absence, quantity, activity or amount (which can be an effective amount) of either a given substance within a clinical or subject-derived sample, including the derivation of qualitative or quantitative concentration levels of such substances, or otherwise evaluating the values or categorization of a subject's non-analyte clinical parameters.

“Negative predictive value” or “NPV” is calculated by TN/(TN+FN) or the true negative fraction of all negative test results. It also is inherently impacted by the prevalence of the disease and pre-test probability of the population intended to be tested.

See, e.g., O'Marcaigh A S, Jacobson R M, “Estimating The Predictive Value Of A Diagnostic Test, How To Prevent Misleading Or Confusing Results,” Clin. Ped. 1993, 32(8): 485-491, which discusses specificity, sensitivity, and positive and negative predictive values of a test, e.g., a clinical diagnostic test. Often, for binary disease state classification approaches using a continuous diagnostic test measurement, the sensitivity and specificity is summarized by Receiver Operating Characteristics (ROC) curves according to Pepe et al, “Limitations of the Odds Ratio in Gauging the Performance of a Diagnostic, Prognostic, or Screening Marker,” Am. J. Epidemiol 2004, 159 (9): 882-890, and summarized by the Area Under the Curve (AUC) or c-statistic, an indicator that allows representation of the sensitivity and specificity of a test, assay, or method over the entire range of test (or assay) cut points with just a single value. See also, e.g., Shultz, “Clinical Interpretation Of Laboratory Procedures,” chapter 14 in Teitz, Fundamentals of Clinical Chemistry, Burtis and Ashwood (eds.), 4^{th} edition 1996, W.B. Saunders Company, pages 192-199; and Zweig et al., “ROC Curve Analysis: An Example Showing The Relationships Among Serum Lipid And Apolipoprotein Concentrations In Identifying Subjects With Coronory Artery Disease,” Clin. Chem., 1992, 38(8): 1425-1428. An alternative approach using likelihood functions, odds ratios, information theory, predictive values, calibration (including goodness-of-fit), and reclassification measurements is summarized according to Cook, “Use and Misuse of the Receiver Operating Characteristic Curve in Risk Prediction,” Circulation 2007, 115: 928-935.

Finally, hazard ratios and absolute and relative risk ratios within subject cohorts defined by a test are a further measurement of clinical accuracy and utility. Multiple methods are frequently used to defining abnormal or disease values, including reference limits, discrimination limits, and risk thresholds.

“Analytical accuracy” refers to the reproducibility and predictability of the measurement process itself, and may be summarized in such measurements as coefficients of variation, and tests of concordance and calibration of the same samples or controls with different times, users, equipment and/or reagents. These and other considerations in evaluating new biomarkers are also summarized in Vasan, 2006.

“Performance” is a term that relates to the overall usefulness and quality of a diagnostic or prognostic test, including, among others, clinical and analytical accuracy, other analytical and process characteristics, such as use characteristics (e.g., stability, ease of use), health economic value, and relative costs of components of the test. Any of these factors may be the source of superior performance and thus usefulness of the test, and may be measured by appropriate “performance metrics,” such as AUC, time to result, shelf life, etc. as relevant.

“Positive predictive value” or “PPV” is calculated by TP/(TP+FP) or the true positive fraction of all positive test results. It is inherently impacted by the prevalence of the disease and pre-test probability of the population intended to be tested.

“Risk” in the context of the present invention, relates to the probability that an event will occur over a specific time period, as in the conversion to metastatic events, and can can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion.

“Risk evaluation,” or “evaluation of risk” in the context of the present invention encompasses making a prediction of the probability, odds, or likelihood that an event or disease state may occur, the rate of occurrence of the event or conversion from one disease state to another, i.e., from a primary tumor to a metastatic tumor or to one at risk of developing a metastatic, or from at risk of a primary metastatic event to a more secondary metastatic event. Risk evaluation can also comprise prediction of future clinical parameters, traditional laboratory risk factor values, or other indices of cancer, either in absolute or relative terms in reference to a previously measured population. The methods of the present invention may be used to make continuous or categorical measurements of the risk of a metastatic tumor thus diagnosing and defining the risk spectrum of a category of subjects defined as being at risk for metastatic tumor. In the categorical scenario, the invention can be used to discriminate between normal and other subject cohorts at higher risk for metastatic tumors. Such differing use may require different DETERMINANT combinations and individualized panels, mathematical algorithms, and/or cut-off points, but be subject to the same aforementioned measurements of accuracy and performance for the respective intended use.

A “sample” in the context of the present invention is a biological sample isolated from a subject and can include, by way of example and not limitation, tissue biopies, whole blood, serum, plasma, blood cells, endothelial cells, lymphatic fluid, ascites fluid, interstitital fluid (also known as “extracellular fluid” and encompasses the fluid found in spaces between cells, including, inter alia, gingival crevicular fluid), bone marrow, cerebrospinal fluid (CSF), saliva, mucous, sputum, sweat, urine, circulating tumor cell, circulating endothelial cell or any other secretion, excretion, or other bodily fluids.

“Sensitivity” is calculated by TP/(TP+FN) or the true positive fraction of disease subjects.

“Specificity” is calculated by TN/(TN+FP) or the true negative fraction of non-disease or normal subjects.

By “statistically significant”, it is meant that the alteration is greater than what might be expected to happen by chance alone (which could be a “false positive”). Statistical significance can be determined by any method known in the art. Commonly used measures of significance include the p-value, which presents the probability of obtaining a result at least as extreme as a given data point, assuming the data point was the result of chance alone. A result is often considered highly significant at a p-value of 0.05 or less.

A “subject” in the context of the present invention is preferably a mammal. The mammal can be a human, non-human primate, mouse, rat, dog, cat, horse, or cow, but are not limited to these examples. Mammals other than humans can be advantageously used as subjects that represent animal models of tumor metastasis. A subject can be male or female. A subject can be one who has been previously diagnosed or identified as having primary tumor or a meastatic tumor, and optionally has already undergone, or is undergoing, a therapeutic intervention for the tumor. Alternatively, a subject can also be one who has not been previously diagnosed as having a metastatic tumor. For example, a subject can be one who exhibits one or more risk factors for a metastatic tumor.

“TN” is true negative, which for a disease state test means classifying a non-disease or normal subject correctly.

“TP” is true positive, which for a disease state test means correctly classifying a disease subject.

“Traditional laboratory risk factors” correspond to biomarkers isolated or derived from subject samples and which are currently evaluated in the clinical laboratory and used in traditional global risk assessment algorithms. Traditional laboratory risk factors for tumor metastasis include for example breslow thickness, ulceration. Proliferative index, tumor-infiltrating lymphocytes. Other traditional laboratory risk factors for tumor metastasis are known to those skilled in the art.

Methods and Uses of the Invention

The methods disclosed herein are used with subjects at risk for developing a metastatic tumor, subjects who may or may not have already been diagnosed with a metastatic tumor and subjects undergoing treatment and/or therapies for a primary tumor or a metastatic tumor. The methods of the present invention can also be used to monitor or select a treatment regimen for a subject who has a primary tumor or a metastatic tumor, and to screen subjects who have not been previously diagnosed as having a metastatic tumor, such as subjects who exhibit risk factors for metastatis. Preferably, the methods of the present invention are used to identify and/or diagnose subjects who are asymptomatic for a metastatic tumor. “Asymptomatic” means not exhibiting the traditional symptoms.

The methods of the present invention may also used to identify and/or diagnose subjects already at higher risk of developing a metastatic tumor based on solely on the traditional risk factors.

A subject having a metastatic tumor can be identified by measuring the amounts (including the presence or absence) of an effective number (which can be two or more) of DETERMINANTS in a subject-derived sample and the amounts are then compared to a reference value. Alterations in the amounts and patterns of expression of biomarkers, such as proteins, polypeptides, nucleic acids and polynucleotides, polymorphisms of proteins, polypeptides, nucleic acids, and polynucleotides, mutated proteins, polypeptides, nucleic acids, and polynucleotides, or alterations in the molecular quantities of metabolites or other analytes in the subject sample compared to the reference value are then identified.

A reference value can be relative to a number or value derived from population studies, including without limitation, such subjects having the same cancer, subject having the same or similar age range, subjects in the same or similar ethnic group, subjects having family histories of cancer, or relative to the starting sample of a subject undergoing treatment for a cancer. Such reference values can be derived from statistical analyses and/or risk prediction data of populations obtained from mathematical algorithms and computed indices of cancer metastasis. Reference DETERMINANT indices can also be constructed and used using algorithms and other methods of statistical and structural classification.

In one embodiment of the present invention, the reference value is the amount of DETERMINANTS in a control sample derived from one or more subjects who are not at risk or at low risk for developing metastatic tumor. In another embodiment of the present invention, the reference value is the amount of DETERMINANTS in a control sample derived from one or more subjects who are asymptomatic and/or lack traditional risk factors for a metastatic tumor. In a further embodiment, such subjects are monitored and/or periodically retested for a diagnostically relevant period of time (“longitudinal studies”) following such test to verify continued absence of a metastatic tumor (disease or event free survival). Such period of time may be one year, two years, two to five years, five years, five to ten years, ten years, or ten or more years from the initial testing date for determination of the reference value. Furthermore, retrospective measurement of DETERMINANTS in properly banked historical subject samples may be used in establishing these reference values, thus shortening the study time required.

A reference value can also comprise the amounts of DETERMINANTS derived from subjects who show an improvement in metastatic risk factors as a result of treatments and/or therapies for the cancer. A reference value can also comprise the amounts of DETERMINANTS derived from subjects who have confirmed disease by known invasive or non-invasive techniques, or are at high risk for developing metastatic tumor, or who have suffered from a metastatic tumor.

In another embodiment, the reference value is an index value or a baseline value. An index value or baseline value is a composite sample of an effective amount of DETERMINANTS from one or more subjects who do not have metastatic tumor, or subjects who are asymptomatic a metastatic. A baseline value can also comprise the amounts of DETERMINANTS in a sample derived from a subject who has shown an improvement in metastatic tumor risk factors as a result of cancer treatments or therapies. In this embodiment, to make comparisons to the subject-derived sample, the amounts of DETERMINANTS are similarly calculated and compared to the index value. Optionally, subjects identified as having metastatic tumor, or being at increased risk of developing a metastatic tumor are chosen to receive a therapeutic regimen to slow the progression the cancer, or decrease or prevent the risk of developing a metastatic tumor.

The progression of a metastatic tumor, or effectiveness of a cancer treatment regimen can be monitored by detecting a DETERMINANT in an effective amount (which may be two or more) of samples obtained from a subject over time and comparing the amount of DETERMINANTS detected. For example, a first sample can be obtained prior to the subject receiving treatment and one or more subsequent samples are taken after or during treatment of the subject. The cancer is considered to be progressive (or, alternatively, the treatment does not prevent progression) if the amount of DETERMINANT changes over time relative to the reference value, whereas the cancer is not progressive if the amount of DETERMINANTS remains constant over time (relative to the reference population, or “constant” as used herein). The term “constant” as used in the context of the present invention is construed to include changes over time with respect to the reference value.

For example, the methods of the invention can be used to discriminate the aggressiveness/and or accessing the stage of the tumor (e.g. Stage I, II, II or IV). This will allow patients to be stratified into high or low risk groups and treated accordingly.

Additionally, therapeutic or prophylactic agents suitable for administration to a particular subject can be identified by detecting a DETERMINANT in an effective amount (which may be two or more) in a sample obtained from a subject, exposing the subject-derived sample to a test compound that determines the amount (which may be two or more) of DETERMINANTS in the subject-derived sample. Accordingly, treatments or therapeutic regimens for use in subjects having a cancer, or subjects at risk for developing metastatic tumor can be selected based on the amounts of DETERMINANTS in samples obtained from the subjects and compared to a reference value. Two or more treatments or therapeutic regimens can be evaluated in parallel to determine which treatment or therapeutic regimen would be the most efficacious for use in a subject to delay onset, or slow progression of the cancer.

The present invention further provides a method for screening for changes in marker expression associated with a metastatic tumor, by determining the amount (which may be two or more) of DETERMINANTS in a subject-derived sample, comparing the amounts of the DETERMINANTS in a reference sample, and identifying alterations in amounts in the subject sample compared to the reference sample.

The present invention further provides a method of treating a patient with a tumor, by identifying a patient with a tumor where an effective amount of DETERMINANTS are altered in a clinically significant manner as measured in a sample from the tumor, an treating the patient with a therapeutic regimen that prevents or reduces tumor metastasis.

Additionally the invention provides a method of selecting a tumor patient in need of adjuvant treatment by assessing the risk of metastasis in the patient by measuring an effective amount of DETERMINANTS where a clinically significant alteration two or more DETERMINANTS in a tumor sample from the patient indicates that the patient is in need of adjuvant treatment.

Information regarding a treatment decision for a tumor patient by obtaining information on an effective amount of DETERMINANTS in a tumor sample from the patient, and selecting a treatment regimen that prevents or reduces tumor metastasis in the patient if two or more DETERMINANTS are altered in a clinically significant manner.

If the reference sample, e.g., a control sample, is from a subject that does not have a metastatic cancer, or if the reference sample reflects a value that is relative to a person that has a high likelihood of rapid progression to a metastatic tumor, a similarity in the amount of the DETERMINANT in the test sample and the reference sample indicates that the treatment is efficacious. However, a difference in the amount of the DETERMINANT in the test sample and the reference sample indicates a less favorable clinical outcome or prognosis.

By “efficacious”, it is meant that the treatment leads to a decrease in the amount or activity of a DETERMINANT protein, nucleic acid, polymorphism, metabolite, or other analyte. Assessment of the risk factors disclosed herein can be achieved using standard clinical protocols. Efficacy can be determined in association with any known method for diagnosing, identifying, or treating anmetastatic disease.

The present invention also provides DETERMINANT panels including one or more DETERMINANTS that are indicative of a general physiological pathway associated with a metastatic For example, one or more DETERMINANTS that can be used to exclude or distinguish between different disease states or sequelae associated with metastatis. A single DETERMINANT may have several of the aforementioned characteristics according to the present invention, and may alternatively be used in replacement of one or more other DETERMINANTS where appropriate for the given application of the invention.

The present invention also comprises a kit with a detection reagent that binds to two or more DETERMINANT proteins, nucleic acids, polymorphisms, metabolites, or other analytes. Also provided by the invention is an array of detection reagents, e.g., antibodies and/or oligonucleotides that can bind to two or more DETERMINANT proteins or nucleic acids, respectively. In one embodiment, the DETERMINANT are proteins and the array contains antibodies that bind an effective amount of DETERMINANTS 1-360 sufficient to measure a statistically significant alteration in DETERMINANT expression compared to a reference value. In another embodiment, the DETERMINANTS are nucleic acids and the array contains oligonucleotides or aptamers that bind an effective amount of DETERMINANTS 1-360 sufficient to measure a statistically significant alteration in DETERMINANT expression compared to a reference value.

In another embodiment, the DETERMINANT are proteins and the array contains antibodies that bind an effective amount of DETERMINANTS 1-25, 41, 61, 62, 63, 66, 74, 96, 99, 103, 126, 135, 137, 138, 177, 190, 210, 212, 217, 218, 227, 239, 261, and 271 sufficient to measure a statistically significant alteration in DETERMINANT expression compared to a reference value. In another embodiment, the DETERMINANTS are nucleic acids and the array contains oligonucleotides or aptamers that bind an effective amount of DETERMINANTS 1-25, 41, 61, 62, 63, 66, 74, 96, 99, 103, 126, 135, 137, 138, 177, 190, 210, 212, 217, 218, 227, 239, 261, and 271 sufficient to measure a statistically significant alteration in DETERMINANT expression compared to a reference value.

Also provided by the present invention is a method for treating one or more subjects at risk for developing a metasatic tumor by detecting the presence of altered amounts of an effective amount of DETERMINANTS present in a sample from the one or more subjects; and treating the one or more subjects with one or more cancer-modulating drugs until altered amounts or activity of the DETERMINANTS return to a baseline value measured in one or more subjects at low risk for developing a metastatic disease, or alternatively, in subjects who do not exhibit any of the traditional risk factors formetastatic disease.

Also provided by the present invention is a method for treating one or more subjects having metastatic tumor by detecting the presence of altered levels of an effective amount of DETERMINANTS present in a sample from the one or more subjects; and treating the one or more subjects with one or more cancer-modulating drugs until altered amounts or activity of the DETERMINANTS return to a baseline value measured in one or more subjects at low risk for developing metastatic tumor.

Also provided by the present invention is a method for evaluating changes in the risk of developing a metastatic tumor in a subject diagnosed with cancer, by detecting an effective amount of DETERMINANTS (which may be two or more) in a first sample from the subject at a first period of time, detecting the amounts of the DETERMINANTS in a second sample from the subject at a second period of time, and comparing the amounts of the DETERMINANTS detected at the first and second periods of time.

Diagnostic and Prognostic Indications of the Invention

The invention allows the diagnosis and prognosis of a metatstatic tumor. The risk of developing a metastatic tumor can be detected by measuring an effective amount of DETERMINANT proteins, nucleic acids, polymorphisms, metabolites, and other analytes (which may be two or more) in a test sample (e.g., a subject derived sample), and comparing the effective amounts to reference or index values, often utilizing mathematical algorithms or formula in order to combine information from results of multiple individual DETERMINANTS and from non-analyte clinical parameters into a single measurement or index. Subjects identified as having an increased risk of an a metastatic tumor can optionally be selected to receive treatment regimens, such as administration of prophylactic or therapeutic compounds to prevent or delay the onset of a metastatic tumor.

The amount of the DETERMINANT protein, nucleic acid, polymorphism, metabolite, or other analyte can be measured in a test sample and compared to the “normal control level,” utilizing techniques such as reference limits, discrimination limits, or risk defining thresholds to define cutoff points and abnormal values. The “normal control level” means the level of one or more DETERMINANTS or combined DETERMINANT indices typically found in a subject not suffering from a metstatic tumor. Such normal control level and cutoff points may vary based on whether a DETERMINANT is used alone or in a formula combining with other DETERMINANTS into an index. Alternatively, the normal control level can be a database of DETERMINANT patterns from previously tested subjects who did not develop a ametastatic tumor over a clinically relevant time horizon.

The present invention may be used to make continuous or categorical measurements of the risk of conversion to a metastatic tumor, thus diagnosing and defining the risk spectrum of a category of subjects defined as at risk for having a metastatic event. In the categorical scenario, the methods of the present invention can be used to discriminate between normal and disease subject cohorts. In other embodiments, the present invention may be used so as to discriminate those at risk for having a metastatic event from those having more rapidly progressing (or alternatively those with a shorter probable time horizon to anmetastatic event) to a metastatic event from those more slowly progressing (or with a longer time horizon to a metastatic event), or those having a metastatic tumor from normal. Such differing use may require different DETERMINANT combinations in individual panel, mathematical algorithm, and/or cut-off points, but be subject to the same aforementioned measurements of accuracy and other performance metrics relevant for the intended use.

Identifying the subject at risk of having a metastatic event enables the selection and initiation of various therapeutic interventions or treatment regimens in order to delay, reduce or prevent that subject's conversion to a metastatic disease state. Levels of an effective amount of DETERMINANT proteins, nucleic acids, polymorphisms, metabolites, or other analytes also allows for the course of treatment of a metastatic disease or metastatic event to be monitored. In this method, a biological sample can be provided from a subject undergoing treatment regimens, e.g., drug treatments, for cancer. If desired, biological samples are obtained from the subject at various time points before, during, or after treatment.

By virtue of determinants' being functionally active, by elucidating its function, subjects with high determinants, for example, can be managed with agents/drugs that preferentially target such pathways, e.g. HOXA1 functioning through TGFβ signaling, thus, high HOXA1 subjects can be treated with TGFβ inhibitors. Or HOXA1 activates CXCR4, a chemokine axis known to be involved in metastasis and reported to act upstream of TGFb, thus, agents/drugs antagonizing CXCR4 can be used.

The present invention can also be used to screen patient or subject populations in any number of settings. For example, a health maintenance organization, public health entity or school health program can screen a group of subjects to identify those requiring interventions, as described above, or for the collection of epidemiological data. Insurance companies (e.g., health, life or disability) may screen applicants in the process of determining coverage or pricing, or existing clients for possible intervention. Data collected in such population screens, particularly when tied to any clinical progession to conditions like cancer or metastatic events, will be of value in the operations of for example, health maintenance organizations, public health programs and insurance companies. Such data arrays or collections can be stored in machine-readable media and used in any number of health-related data management systems to provide improved healthcare services, cost effective healthcare, improved insurance operation, etc. See, for example, U.S. Patent Application No. 2002/0038227; U.S. Patent Application No. US 2004/0122296; U.S. Patent Application No. US 2004/0122297; and U.S. Pat. No. 5,018,067. Such systems can access the data directly from internal data storage or remotely from one or more data storage sites as further detailed herein.

A machine-readable storage medium can comprise a data storage material encoded with machine readable data or data arrays which, when using a machine programmed with instructions for using said data, is capable of use for a variety of purposes, such as, without limitation, subject information relating to metastatic disease risk factors over time or in response drug therapies. Measurements of effective amounts of the biomarkers of the invention and/or the resulting evaluation of risk from those biomarkers can implemented in computer programs executing on programmable computers, comprising, inter alia, a processor, a data storage system (including volatile and non-volatile memory and/or storage elements), at least one input device, and at least one output device. Program code can be applied to input data to perform the functions described above and generate output information. The output information can be applied to one or more output devices, according to methods known in the art. The computer may be, for example, a personal computer, microcomputer, or workstation of conventional design.

Each program can be implemented in a high level procedural or object oriented programming language to communicate with a computer system. However, the programs can be implemented in assembly or machine language, if desired. The language can be a compiled or interpreted language. Each such computer program can be stored on a storage media or device (e.g., ROM or magnetic diskette or others as defined elsewhere in this disclosure) readable by a general or special purpose programmable computer, for configuring and operating the computer when the storage media or device is read by the computer to perform the procedures described herein. The health-related data management system of the invention may also be considered to be implemented as a computer-readable storage medium, configured with a computer program, where the storage medium so configured causes a computer to operate in a specific and predefined manner to perform various functions described herein.

Levels of an effective amount of DETERMINANT proteins, nucleic acids, polymorphisms, metabolites, or other analytes can then be determined and compared to a reference value, e.g. a control subject or population whose metastatic state is known or an index value or baseline value. The reference sample or index value or baseline value may be taken or derived from one or more subjects who have been exposed to the treatment, or may be taken or derived from one or more subjects who are at low risk of developing cancer or a metstatic event, or may be taken or derived from subjects who have shown improvements in as a result of exposure to treatment. Alternatively, the reference sample or index value or baseline value may be taken or derived from one or more subjects who have not been exposed to the treatment. For example, samples may be collected from subjects who have received initial treatment for cancer or a metastatic event and subsequent treatment for cancer or a metastatic event to monitor the progress of the treatment. A reference value can also comprise a value derived from risk prediction algorithms or computed indices from population studies such as those disclosed herein.

The DETERMINANTS of the present invention can thus be used to generate a “reference DETERMINANT profile” of those subjects who do not have cancer or are not at risk of having a metastaic event, and would not be expected to develop cancer or a metastatic event. The DETERMINANTS disclosed herein can also be used to generate a “subject DETERMINANT profile” taken from subjects who have cancer or are at risk for having a metastatic event. The subject DETERMINANT profiles can be compared to a reference DETERMINANT profile to diagnose or identify subjects at risk for developing cancer or a metastatic event, to monitor the progression of disease, as well as the rate of progression of disease, and to monitor the effectiveness of treatment modalities. The reference and subject DETERMINANT profiles of the present invention can be contained in a machine-readable medium, such as but not limited to, analog tapes like those readable by a VCR, CD-ROM, DVD-ROM, USB flash media, among others. Such machine-readable media can also contain additional test results, such as, without limitation, measurements of clinical parameters and traditional laboratory risk factors. Alternatively or additionally, the machine-readable media can also comprise subject information such as medical history and any relevant family history. The machine-readable media can also contain information relating to other disease-risk algorithms and computed indices such as those described herein.

Differences in the genetic makeup of subjects can result in differences in their relative abilities to metabolize various drugs, which may modulate the symptoms or risk factors of cancer or metastatic events. Subjects that have cancer, or at risk for developing cancer or a metastatic event can vary in age, ethnicity, and other parameters. Accordingly, use of the DETERMINANTS disclosed herein, both alone and together in combination with known genetic factors for drug metabolism, allow for a pre-determined level of predictability that a putative therapeutic or prophylactic to be tested in a selected subject will be suitable for treating or preventing cancer or a metastatic event in the subject.

To identify therapeutics or drugs that are appropriate for a specific subject, a test sample from the subject can also be exposed to a therapeutic agent or a drug, and the level of one or more of DETERMINANT proteins, nucleic acids, polymorphisms, metabolites or other analytes can be determined. The level of one or more DETERMINANTS can be compared to sample derived from the subject before and after treatment or exposure to a therapeutic agent or a drug, or can be compared to samples derived from one or more subjects who have shown improvements in risk factors (e.g., clinical parameters or traditional laboratory risk factors) as a result of such treatment or exposure.

A subject cell (i.e., a cell isolated from a subject) can be incubated in the presence of a candidate agent and the pattern of DETERMINANT expression in the test sample is measured and compared to a reference profile, e.g., a metastatic disease reference expression profile or a non-disease reference expression profile or an index value or baseline value. The test agent can be any compound or composition or combination thereof, including, dietary supplements. For example, the test agents are agents frequently used in cancer treatment regimens and are described herein.

The aforementioned methods of the invention can be used to evaluate or monitor the progression and/or improvement of subjects who have been diagnosed with a cancer, and who have undergone surgical interventions.

Performance and Accuracy Measures of the Invention

The performance and thus absolute and relative clinical usefulness of the invention may be assessed in multiple ways as noted above. Amongst the various assessments of performance, the invention is intended to provide accuracy in clinical diagnosis and prognosis. The accuracy of a diagnostic or prognostic test, assay, or method concerns the ability of the test, assay, or method to distinguish between subjects having cancer, or at risk for cancer or a metastatic event, is based on whether the subjects have, a “significant alteration” (e.g., clinically significant “diagnostically significant) in the levels of a DETERMINANT. By “effective amount” it is meant that the measurement of an appropriate number of DETERMINANTS (which may be one or more) to produce a “significant alteration,” (e.g. level of expression or activity of a DETERMINANT) that is different than the predetermined cut-off point (or threshold value) for that DETERMINANT(S) and therefore indicates that the subject has cancer or is at risk for having a metastatic event for which the DETERMINANT(S) is a determinant. The difference in the level of DETERMINANT between normal and abnormal is preferably statistically significant. As noted below, and without any limitation of the invention, achieving statistical significance, and thus the preferred analytical, diagnostic, and clinical accuracy, generally but not always requires that combinations of several DETERMINANTS be used together in panels and combined with mathematical algorithms in order to achieve a statistically significant DETERMINANT index.

In the categorical diagnosis of a disease state, changing the cut point or threshold value of a test (or assay) usually changes the sensitivity and specificity, but in a qualitatively inverse relationship. Therefore, in assessing the accuracy and usefulness of a proposed medical test, assay, or method for assessing a subject's condition, one should always take both sensitivity and specificity into account and be mindful of what the cut point is at which the sensitivity and specificity are being reported because sensitivity and specificity may vary significantly over the range of cut points. Use of statistics such as AUC, encompassing all potential cut point values, is preferred for most categorical risk measures using the invention, while for continuous risk measures, statistics of goodness-of-fit and calibration to observed results or other gold standards, are preferred.

By predetermined level of predictability it is meant that the method provides an acceptable level of clininal or diagnostic accuracy. Using such statistics, an “acceptable degree of diagnostic accuracy”, is herein defined as a test or assay (such as the test of the invention for determining the clinically significant presence of DETERMINANTS, which thereby indicates the presence of cancer and/or a risk of having a metastatic event) in which the AUC (area under the ROC curve for the test or assay) is at least 0.60, desirably at least 0.65, more desirably at least 0.70, preferably at least 0.75, more preferably at least 0.80, and most preferably at least 0.85.

By a “very high degree of diagnostic accuracy”, it is meant a test or assay in which the AUC (area under the ROC curve for the test or assay) is at least 0.75, 0.80, desirably at least 0.85, more desirably at least 0.875, preferably at least 0.90, more preferably at least 0.925, and most preferably at least 0.95.

Alternatively, the methods predict the presence or absence of a cancer, metastatic cancer or response to therapy with at least 75% accuracy, more preferably 80%, 85%, 90%, 95%, 97%, 98%, 99% or greater accuracy.

The predictive value of any test depends on the sensitivity and specificity of the test, and on the prevalence of the condition in the population being tested. This notion, based on Bayes' theorem, provides that the greater the likelihood that the condition being screened for is present in an individual or in the population (pre-test probability), the greater the validity of a positive test and the greater the likelihood that the result is a true positive. Thus, the problem with using a test in any population where there is a low likelihood of the condition being present is that a positive result has limited value (i.e., more likely to be a false positive). Similarly, in populations at very high risk, a negative test result is more likely to be a false negative.

As a result, ROC and AUC can be misleading as to the clinical utility of a test in low disease prevalence tested populations (defined as those with less than 1% rate of occurrences (incidence) per annum, or less than 10% cumulative prevalence over a specified time horizon). Alternatively, absolute risk and relative risk ratios as defined elsewhere in this disclosure can be employed to determine the degree of clinical utility. Populations of subjects to be tested can also be categorized into quartiles by the test's measurement values, where the top quartile (25% of the population) comprises the group of subjects with the highest relative risk for developing cancer or metastatic event, and the bottom quartile comprising the group of subjects having the lowest relative risk for developing cancer or a metastatic event. Generally, values derived from tests or assays having over 2.5 times the relative risk from top to bottom quartile in a low prevalence population are considered to have a “high degree of diagnostic accuracy,” and those with five to seven times the relative risk for each quartile are considered to have a “very high degree of diagnostic accuracy.” Nonetheless, values derived from tests or assays having only 1.2 to 2.5 times the relative risk for each quartile remain clinically useful are widely used as risk factors for a disease; such is the case with total cholesterol and for many inflammatory biomarkers with respect to their prediction of future metastatic events. Often such lower diagnostic accuracy tests must be combined with additional parameters in order to derive meaningful clinical thresholds for therapeutic intervention, as is done with the aforementioned global risk assessment indices.

A health economic utility function is an yet another means of measuring the performance and clinical value of a given test, consisting of weighting the potential categorical test outcomes based on actual measures of clinical and economic value for each. Health economic performance is closely related to accuracy, as a health economic utility function specifically assigns an economic value for the benefits of correct classification and the costs of misclassification of tested subjects. As a performance measure, it is not unusual to require a test to achieve a level of performance which results in an increase in health economic value per test (prior to testing costs) in excess of the target price of the test.

In general, alternative methods of determining diagnostic accuracy are commonly used for continuous measures, when a disease category or risk category (such as those at risk for having anmetastatic event) has not yet been clearly defined by the relevant medical societies and practice of medicine, where thresholds for therapeutic use are not yet established, or where there is no existing gold standard for diagnosis of the pre-disease. For continuous measures of risk, measures of diagnostic accuracy for a calculated index are typically based on curve fit and calibration between the predicted continuous value and the actual observed values (or a historical index calculated value) and utilize measures such as R squared, Hosmer-Lemeshow P-value statistics and confidence intervals. It is not unusual for predicted values using such algorithms to be reported including a confidence interval (usually 90% or 95% CI) based on a historical observed cohort's predictions, as in the test for risk of future breast cancer recurrence commercialized by Genomic Health, Inc. (Redwood City, Calif.).

In general, by defining the degree of diagnostic accuracy, i.e., cut points on a ROC curve, defining an acceptable AUC value, and determining the acceptable ranges in relative concentration of what constitutes an effective amount of the DETERMINANTS of the invention allows for one of skill in the art to use the DETERMINANTS to identify, diagnose, or prognose subjects with a pre-determined level of predictability and performance.

Risk Markers of the Invention (DETERMINANTS)

The biomarkers and methods of the present invention allow one of skill in the art to identify, diagnose, or otherwise assess those subjects who do not exhibit any symptoms of cancer or a metastatic event, but who nonetheless may be at risk for developing cancer or a metastatic event.

One thousand five hundred and ninety-three biomarkers have been identified as being found to have altered or modified presence or concentration levels in subjects who have metastatic disease.

Table I comprises the three hundred and sixty (360) overexpressed/amplified or downregulated/deleted phentotype driven evoluntionary-concernved DETERMINANTS of the present invention. DETERMINANTS 1-25, 41, 61, 62, 63, 66, 74, 96, 99, 103, 126, 135, 137, 138, 177, 190, 210, 212, 217, 218, 227, 239, 261, and 271 have been identified as pro-invasion determinants.

TABLE I

| | | Determinant |
|---|---|---|
| Gene ID | Gene Symbol | No: |
| 54 | ACP5 | 1 |
| 54443 | ANLN | 2 |
| 55723 | ASF1B | 3 |
| 23397 | BRRN1 | 4 |
| 699 | BUB1 | 5 |
| 79019 | CENPM | 6 |
| 55635 | DEPDC1 | 7 |
| 64123 | ELTD1 | 8 |
| 6624 | FSCN1 | 9 |
| 64151 | HCAP-G | 10 |
| 3146 | HMGB1 | 11 |
| 3198 | HOXA1 | 12 |
| 3297 | HSF1 | 13 |
| 23421 | ITGB3BP | 14 |
| 10112 | KIF20A | 15 |
| 11004 | KIF2C | 16 |
| 10403 | KNTC2 | 17 |
| 4176 | MCM7 | 18 |
| 10797 | MTHFD2 | 19 |
| 11156 | PTP4A3 | 20 |
| 6045 | RNF2 | 21 |
| 10615 | SPAG5 | 22 |
| 11065 | UBE2C | 23 |
| 51377 | UCHL5 | 24 |
| 11326 | VSIG4 | 25 |
| 79575 | ABHD8 | 26 |
| 1636 | ACE | 27 |
| 8038 | ADAM12 | 28 |
| 101 | ADAM8 | 29 |
| 23600 | AMACR | 30 |
| 80833 | APOL3 | 31 |
| 410 | ARSA | 32 |
| 22901 | ARSG | 33 |
| 259266 | ASPM | 34 |
| 477 | ATP1A2 | 35 |
| 6790 | AURKA | 36 |
| 9212 | AURKB | 37 |
| 26053 | AUTS2 | 38 |
| 627 | BDNF | 39 |
| 638 | BIK | 40 |
| 332 | BIRC5 | 41 |
| 672 | BRCA1 | 42 |
| 701 | BUB1B | 43 |
| 80135 | BXDC5 | 44 |
| 29902 | C12ORF24 | 45 |
| 55839 | C16ORF60 | 46 |
| 56942 | C16ORF61 | 47 |
| 116496 | C1ORF24 | 48 |
| 719 | C3AR1 | 49 |
| 57002 | C7ORF36 | 50 |
| 84933 | C8ORF76 | 51 |
| 152007 | C9ORF19 | 52 |
| 781 | CACNA2D1 | 153 |
| 857 | CAV1 | 54 |
| 6357 | CCL13 | 55 |
| 6347 | CCL2 | 56 |
| 6354 | CCL7 | 57 |
| 890 | CCNA2 | 58 |
| 947 | CD34 | 59 |
| 948 | CD36 | 60 |
| 983 | CDC2 | 61 |
| 991 | CDC20 | 62 |
| 995 | CDC25C | 63 |
| 990 | CDC6 | 64 |
| 8317 | CDC7 | 65 |
| 83540 | CDCA1 | 66 |
| 83461 | CDCA3 | 67 |
| 55536 | CDCA7L | 68 |
| 81620 | CDT1 | 69 |
| 1058 | CENPA | 70 |
| 1062 | CENPE | 71 |
| 1063 | CENPF | 72 |
| 55165 | CEP55 | 73 |
| 23177 | CEP68 | 74 |
| 1070 | CETN3 | 75 |
| 1111 | CHEK1 | 76 |
| 26586 | CKAP2 | 77 |
| 1163 | CKS1B | 78 |
| 1164 | CKS2 | 79 |
| 1180 | CLCN1 | 80 |
| 7122 | CLDN5 | 81 |
| 23601 | CLEC5A | 82 |
| 9918 | CNAP1 | 83 |
| 10664 | CTCF | 84 |
| 1565 | CYP2D6 | 85 |
| 1601 | DAB2 | 86 |
| 10926 | DBF4 | 87 |
| 23564 | DDAH2 | 88 |
| 1719 | DHFR | 89 |
| 55355 | DKFZP762E1312 | 90 |
| 27122 | DKK3 | 91 |
| 9787 | DLG7 | 92 |
| 1769 | DNAH8 | 93 |
| 30836 | DNTTIP2 | 94 |
| 51514 | DTL | 95 |
| 1854 | DUT | 96 |
| 1894 | ECT2 | 97 |
| 51162 | EGFL7 | 98 |
| 56943 | ENY2 | 99 |
| 54749 | EPDR1 | 100 |
| 51327 | ERAF | 101 |
| 2115 | ETV1 | 102 |
| 2131 | EXT1 | 103 |
| 2162 | F13A1 | 104 |
| 51647 | FAM96B | 105 |
| 2230 | FDX1 | 106 |
| 2235 | FECH | 107 |
| 63979 | FIGNL1 | 108 |
| 51303 | FKBP11 | 109 |
| 2289 | FKBP5 | 110 |
| 55110 | FLJ10292 | 111 |
| 79805 | FLJ12505 | 112 |
| 84935 | FLJ14834 | 113 |
| 54908 | FLJ20364 | 114 |
| 54962 | FLJ20516 | 115 |
| 2350 | FOLR2 | 116 |
| 2305 | FOXM1 | 117 |
| 2530 | FUT8 | 118 |
| 51809 | GALNT7 | 119 |
| 64096 | GFRA4 | 120 |
| 2740 | GLP1R | 121 |
| 51053 | GMNN | 122 |
| 2775 | GNAO1 | 123 |
| 2792 | GNGT1 | 124 |
| 4076 | GPIAP1 | 125 |
| 2894 | GRID1 | 126 |
| 2936 | GSR | 127 |
| 2966 | GTF2H2 | 128 |
| 51512 | GTSE1 | 129 |
| 3045 | HBD | 130 |
| 50810 | HDGFRP3 | 131 |
| 3082 | HGF | 132 |
| 3012 | HIST1H2AB | 133 |
| 3142 | HLX1 | 134 |
| 3148 | HMGB2 | 135 |
| 3161 | HMMR | 136 |
| 10236 | HNRPR | 137 |
| 10247 | HRSP12 | 138 |
| 3313 | HSPA9B | 139 |
| 51501 | HSPC138 | 140 |
| 10808 | HSPH1 | 141 |
| 25998 | IBTK | 142 |
| 3384 | ICAM2 | 143 |
| 80173 | IFT74 | 144 |
| 3570 | IL6R | 145 |
| 3684 | ITGAM | 146 |
| 6453 | ITSN1 | 147 |
| 10008 | KCNE3 | 148 |
| 3776 | KCNK2 | 149 |
| 9768 | KIAA0101 | 150 |
| 9694 | KIAA0103 | 151 |
| 56243 | KIAA1217 | 152 |
| 84629 | KIAA1856 | 153 |
| 3832 | KIF11 | 154 |
| 81930 | KIF18A | 155 |
| 3833 | KIFC1 | 156 |
| 55220 | KLHDC8A | 157 |
| 3912 | LAMB1 | 158 |
| 3915 | LAMC1 | 159 |
| 55915 | LANCL2 | 160 |
| 11025 | LILRB3 | 161 |
| 4005 | LMO2 | 162 |
| 150084 | LOC150084 | 163 |
| 345711 | LOC345711 | 164 |
| 91614 | LOC91614 | 165 |
| 26018 | LRIG1 | 166 |
| 54892 | LUZP5 | 167 |
| 4085 | MAD2L1 | 168 |
| 6300 | MAPK12 | 169 |
| 4147 | MATN2 | 170 |
| 4172 | MCM3 | 171 |
| 4174 | MCM5 | 172 |
| 4175 | MCM6 | 173 |
| 9833 | MELK | 174 |
| 4232 | MEST | 175 |
| 4233 | MET | 176 |
| 85014 | MGC14141 | 177 |
| 79971 | MIER1 | 178 |
| 4288 | MKI67 | 179 |
| 8028 | MLLT10 | 180 |
| 4317 | MMP8 | 181 |
| 4318 | MMP9 | 182 |
| 4353 | MPO | 183 |
| 51678 | MPP6 | 184 |
| 219928 | MRGPRF | 185 |
| 64968 | MRPS6 | 186 |
| 10335 | MRVI1 | 187 |
| 10232 | MSLN | 188 |
| 4600 | MX2 | 189 |
| 4678 | NASP | 190 |
| 4751 | NEK2 | 191 |
| 23530 | NNT | 192 |
| 4846 | NOS3 | 193 |
| 4855 | NOTCH4 | 194 |
| 84955 | NUDCD1 | 195 |
| 11163 | NUDT4 | 196 |
| 53371 | NUP54 | 197 |
| 4928 | NUP98 | 198 |
| 51203 | NUSAP1 | 199 |
| 4999 | ORC2L | 200 |
| 116039 | OSR2 | 201 |
| 5019 | OXCT1 | 202 |
| 56288 | PARD3 | 203 |
| 55872 | PBK | 204 |
| 11333 | PDAP1 | 205 |
| 5138 | PDE2A | 206 |
| 5156 | PDGFRA | 207 |
| 5175 | PECAM1 | 208 |
| 5218 | PFTK1 | 209 |
| 25776 | PGEA1 | 210 |
| 26227 | PHGDH | 211 |
| 83483 | PLVAP | 212 |
| 57125 | PLXDC1 | 213 |
| 5425 | POLD2 | 214 |
| 5427 | POLE2 | 215 |
| 5446 | PON3 | 216 |
| 5557 | PRIM1 | 217 |
| 5558 | PRIM2A | 218 |
| 5578 | PRKCA | 219 |
| 23627 | PRND | 220 |
| 9265 | PSCD3 | 221 |
| 5743 | PTGS2 | 222 |
| 5885 | RAD21 | 223 |
| 5888 | RAD51 | 224 |
| 5889 | RAD51C | 225 |
| 3516 | RBPSUH | 226 |
| 5965 | RECQL | 227 |
| 5984 | RFC4 | 228 |
| 5985 | RFC5 | 229 |
| 23179 | RGL1 | 230 |
| 64407 | RGS18 | 231 |
| 5997 | RGS2 | 232 |
| 8490 | RGS5 | 233 |
| 9584 | RNPC2 | 234 |
| 6091 | ROBO1 | 235 |
| 6118 | RPA2 | 236 |
| 6119 | RPA3 | 237 |
| 6222 | RPS18 | 238 |
| 6236 | RRAD | 239 |
| 22800 | RRAS2 | 240 |
| 6240 | RRM1 | 241 |
| 6241 | RRM2 | 242 |
| 340419 | RSPO2 | 243 |
| 10371 | SEMA3A | 244 |
| 143686 | SESN3 | 245 |
| 85358 | SHANK3 | 246 |
| 79801 | SHCBP1 | 247 |
| 8036 | SHOC2 | 248 |
| 23517 | SKIV2L2 | 249 |
| 7884 | SLBP | 250 |
| 6509 | SLC1A4 | 251 |
| 115286 | SLC25A26 | 252 |
| 6526 | SLC5A3 | 253 |
| 8467 | SMARCA5 | 254 |
| 8243 | SMC1L1 | 255 |
| 10592 | SMC2L1 | 256 |
| 10051 | SMC4L1 | 257 |
| 6629 | SNRPB2 | 258 |
| 64321 | SOX17 | 259 |
| 6662 | SOX9 | 260 |
| 57405 | SPBC25 | 261 |
| 60559 | SPCS3 | 262 |
| 6741 | SSB | 263 |
| 6742 | SSBP1 | 264 |
| 26872 | STEAP1 | 265 |
| 6788 | STK3 | 266 |
| 10460 | TACC3 | 267 |
| 23435 | TARDBP | 268 |
| 25771 | TBC1D22A | 269 |
| 6899 | TBX1 | 270 |
| 7052 | TGM2 | 271 |
| 90390 | THRAP6 | 272 |
| 8914 | TIMELESS | 273 |
| 7077 | TIMP2 | 274 |
| 7083 | TK1 | 275 |
| 55273 | TMEM100 | 276 |
| 55161 | TMEM33 | 277 |
| 55706 | TMEM48 | 278 |
| 54543 | TOMM7 | 279 |
| 7153 | TOP2A | 280 |
| 22974 | TPX2 | 281 |
| 54209 | TREM2 | 282 |
| 4591 | TRIM37 | 283 |
| 9319 | TRIP13 | 284 |
| 95681 | TSGA14 | 285 |
| 7371 | UCK2 | 286 |
| 83878 | USHBP1 | 287 |
| 10894 | XLKD1 | 288 |
| 51776 | ZAK | 289 |
| 221527 | ZBTB12 | 290 |
| 346171 | ZFP57 | 291 |
| 23414 | ZFPM2 | 292 |
| 79830 | ZMYM1 | 293 |
| 7705 | ZNF146 | 294 |
| 84858 | ZNF503 | 295 |
| 79026 | AHNAK | 296 |
| 360 | AQP3 | 297 |
| 622 | BDH1 | 298 |
| 219738 | C10ORF35 | 299 |
| 726 | CAPN5 | 300 |
| 999 | CDH1 | 301 |
| 51673 | CGI-38 | 302 |
| 1159 | CKMT1B | 303 |
| 85445 | CNTNAP4 | 304 |
| 1303 | COL12A1 | 305 |
| 9244 | CRLF1 | 306 |
| 1410 | CRYAB | 307 |
| 1428 | CRYM | 308 |
| 113878 | DTX2 | 309 |
| 10278 | EFS | 310 |
| 79993 | ELOVL7 | 311 |
| 2041 | EPHA1 | 312 |
| 2045 | EPHA7 | 313 |
| 2051 | EPHB6 | 314 |
| 10205 | EVA1 | 315 |
| 2125 | EVPL | 316 |
| 2159 | F10 | 317 |
| 375061 | FAM89A | 318 |
| 8857 | FCGBP | 319 |
| 2261 | FGFR3 | 320 |
| 56776 | FMN2 | 321 |
| 2770 | GNAI1 | 322 |
| 7107 | GPR137B | 323 |
| 64388 | GREM2 | 324 |
| 3098 | HK1 | 325 |
| 688 | KLF5 | 326 |
| 5655 | KLK10 | 327 |
| 11202 | KLK8 | 328 |
| 10748 | KLRA1 | 329 |
| 10219 | KLRG1 | 330 |
| 4135 | MAP6 | 331 |
| 5603 | MAPK13 | 332 |
| 4312 | MMP1 | 333 |
| 4486 | MST1R | 334 |
| 4692 | NDN | 335 |
| 5092 | PCBD1 | 336 |
| 10158 | PDZK1IP1 | 337 |
| 5317 | PKP1 | 338 |
| 26499 | PLEK2 | 339 |
| 58473 | PLEKHB1 | 340 |
| 5366 | PMAIP1 | 341 |
| 79983 | POF1B | 342 |
| 5453 | POU3F1 | 343 |
| 5579 | PRKCB1 | 344 |
| 5745 | PTHR1 | 345 |
| 5792 | PTPRF | 346 |
| 57111 | RAB25 | 347 |
| 6095 | RORA | 348 |
| 6337 | SCNN1A | 349 |
| 6382 | SDC1 | 350 |
| 5268 | SERPINB5 | 351 |
| 11254 | SLC6A14 | 352 |
| 6578 | SLCO2A1 | 353 |
| 6586 | SLIT3 | 354 |
| 10653 | SPINT2 | 355 |
| 6768 | ST14 | 356 |
| 7070 | THY1 | 357 |
| 23650 | TRIM29 | 358 |
| 23555 | TSPAN15 | 359 |
| 11197 | WIF1 | 360 |

One skilled in the art will recognize that the DETERMINANTS presented herein encompasses all forms and variants, including but not limited to, polymorphisms, isoforms, mutants, derivatives, precursors including nucleic acids and pro-proteins, cleavage products, receptors (including soluble and transmembrane receptors), ligands, protein-ligand complexes, and post-translationally modified variants (such as cross-linking or glycosylation), fragments, and degradation products, as well as any multi-unit nucleic acid, protein, and glycoprotein structures comprised of any of the DETERMINANTS as constituent sub-units of the fully assembled structure.

One skilled in the art will note that the above listed DETERMINANTS come from a diverse set of physiological and biological pathways, including many which are not commonly accepted to be related to metastatic disease. These groupings of different DETERMINANTS, even within those high significance segments, may presage differing signals of the stage or rate of the progression of the disease. Such distinct groupings of DETERMINANTS may allow a more biologically detailed and clinically useful signal from the DETERMINANTS as well as opportunities for pattern recognition within the DETERMINANT algorithms combining the multiple DETERMINANT signals.

The present invention concerns, in one aspect, a subset of DETERMINANTS; other DETERMINANTS and even biomarkers which are not listed in the above Table 1, but related to these physiological and biological pathways, may prove to be useful given the signal and information provided from these studies. To the extent that other biomarker pathway participants (i.e., other biomarker participants in common pathways with those biomarkers contained within the list of DETERMINANTS in the above Table 1) are also relevant pathway participants in cancer or a metastatic event, they may be functional equivalents to the biomarkers, such as for example CXCR4 thus far disclosed in Table 1. These other pathway participants are also considered DETERMINANTS in the context of the present invention, provided they additionally share certain defined characteristics of a good biomarker, which would include both involvement in the herein disclosed biological processes and also analytically important characteristics such as the bioavailability of said biomarkers at a useful signal to noise ratio, and in a useful and accessible sample matrix such as blood serum. Such requirements typically limit the diagnostic usefulness of many members of a biological pathway, and frequently occurs only in pathway members that constitute secretory substances, those accessible on the plasma membranes of cells, as well as those that are released into the serum upon cell death, due to apoptosis or for other reasons such as endothelial remodeling or other cell turnover or cell necrotic processes, whether or not they are related to the disease progression of cancer or metastatic event. However, the remaining and future biomarkers that meet this high standard for DETERMINANTS are likely to be quite valuable.

Furthermore, other unlisted biomarkers will be very highly correlated with the biomarkers listed as DETERMINANTS in Table 1 (for the purpose of this application, any two variables will be considered to be “very highly correlated” when they have a Coefficient of Determination (R²) of 0.5 or greater). The present invention encompasses such functional and statistical equivalents to the aforementioned DETERMINANTS. Furthermore, the statistical utility of such additional DETERMINANTS is substantially dependent on the cross-correlation between multiple biomarkers and any new biomarkers will often be required to operate within a panel in order to elaborate the meaning of the underlying biology.

One or more, preferably two or more of the listed DETERMINANTS can be detected in the practice of the present invention. For example, two (2), three (3), four (4), five (5), ten (10), fifteen (15), twenty (20), forty (40), fifty (50), seventy-five (75), one hundred (100), one hundred and twenty five (125), one hundred and fifty (150), one hundred and seventy-five (175), two hundred (200), two hundred and ten (210), two hundred and twenty (220), two hundred and thirty (230), two hundred and forty (240), two hundred and fifty (250), two hundred and sixty (260) or more, two hundred and seventy (270) or more, two hundred and eighty (280) or more, two hundred and ninety (290) or more, DETERMINANTS can be detected.

In some aspects, all 360 DETERMINANTS listed herein can be detected. Preferred ranges from which the number of DETERMINANTS can be detected include ranges bounded by any minimum selected from between one and 360, particularly two, five, ten, twenty, fifty, seventy-five, one hundred, one hundred and twenty five, one hundred and fifty, one hundred and seventy-five, two hundred, two hundred and ten, two hundred and twenty, two hundred and thirty, two hundred and forty, two hundred and fifty, paired with any maximum up to the total known DETERMINANTS, particularly five, ten, twenty, fifty, and seventy-five. Particularly preferred ranges include two to five (2-5), two to ten (2-10), two to fifty (2-50), two to seventy-five (2-75), two to one hundred (2-100), five to ten (5-10), five to twenty (5-20), five to fifty (5-50), five to seventy-five (5-75), five to one hundred (5-100), ten to twenty (10-20), ten to fifty (10-50), ten to seventy-five (10-75), ten to one hundred (10-100), twenty to fifty (20-50), twenty to seventy-five (20-75), twenty to one hundred (20-100), fifty to seventy-five (50-75), fifty to one hundred (50-100), one hundred to one hundred and twenty-five (100-125), one hundred and twenty-five to one hundred and fifty (125-150), one hundred and fifty to one hundred and seventy five (150-175), one hundred and seventy-five to two hundred (175-200), two hundred to two hundred and ten (200-210), two hundred and ten to two hundred and twenty (210-220), two hundred and twenty to two hundred and thirty (220-230), two hundred and thirty to two hundred and forty (230-240), two hundred and forty to two hundred and fifty (240-250), two hundred and fifty to two hundred and sixty (250-260).

Construction of DETERMINANT Panels

Groupings of DETERMINANTS can be included in “panels.” A “panel” within the context of the present invention means a group of biomarkers (whether they are DETERMINANTS, clinical parameters, or traditional laboratory risk factors) that includes more than one DETERMINANT. A panel can also comprise additional biomarkers, e.g., clinical parameters, traditional laboratory risk factors, known to be present or associated with cancer or cancer metastatis, in combination with a selected group of the DETERMINANTS listed in Table 1.

As noted above, many of the individual DETERMINANTS, clinical parameters, and traditional laboratory risk factors listed, when used alone and not as a member of a multi-biomarker panel of DETERMINANTS, have little or no clinical use in reliably distinguishing individual normal subjects, subjects at risk for having a metastatic event, and subjects having cancer from each other in a selected general population, and thus cannot reliably be used alone in classifying any subject between those three states. Even where there are statistically significant differences in their mean measurements in each of these populations, as commonly occurs in studies which are sufficiently powered, such biomarkers may remain limited in their applicability to an individual subject, and contribute little to diagnostic or prognostic predictions for that subject. A common measure of statistical significance is the p-value, which indicates the probability that an observation has arisen by chance alone; preferably, such p-values are 0.05 or less, representing a 5% or less chance that the observation of interest arose by chance. Such p-values depend significantly on the power of the study performed.

Despite this individual DETERMINANT performance, and the general performance of formulas combining only the traditional clinical parameters and few traditional laboratory risk factors, the present inventors have noted that certain specific combinations of two or more DETERMINANTS can also be used as multi-biomarker panels comprising combinations of DETERMINANTS that are known to be involved in one or more physiological or biological pathways, and that such information can be combined and made clinically useful through the use of various formulae, including statistical classification algorithms and others, combining and in many cases extending the performance characteristics of the combination beyond that of the individual DETERMINANTS. These specific combinations show an acceptable level of diagnostic accuracy, and, when sufficient information from multiple DETERMINANTS is combined in a trained formula, often reliably achieve a high level of diagnostic accuracy transportable from one population to another.

The general concept of how two less specific or lower performing DETERMINANTS are combined into novel and more useful combinations for the intended indications, is a key aspect of the invention. Multiple biomarkers can often yield better performance than the individual components when proper mathematical and clinical algorithms are used; this is often evident in both sensitivity and specificity, and results in a greater AUC. Secondly, there is often novel unperceived information in the existing biomarkers, as such was necessary in order to achieve through the new formula an improved level of sensitivity or specificity. This hidden information may hold true even for biomarkers which are generally regarded to have suboptimal clinical performance on their own. In fact, the suboptimal performance in terms of high false positive rates on a single biomarker measured alone may very well be an indicator that some important additional information is contained within the biomarker results information which would not be elucidated absent the combination with a second biomarker and a mathematical formula.

Several statistical and modeling algorithms known in the art can be used to both assist in DETERMINANT selection choices and optimize the algorithms combining these choices. Statistical tools such as factor and cross-biomarker correlation/covariance analyses allow more rationale approaches to panel construction. Mathematical clustering and classification tree showing the Euclidean standardized distance between the DETERMINANTS can be advantageously used. Pathway informed seeding of such statistical classification techniques also may be employed, as may rational approaches based on the selection of individual DETERMINANTS based on their participation across in particular pathways or physiological functions.

Ultimately, formula such as statistical classification algorithms can be directly used to both select DETERMINANTS and to generate and train the optimal formula necessary to combine the results from multiple DETERMINANTS into a single index. Often, techniques such as forward (from zero potential explanatory parameters) and backwards selection (from all available potential explanatory parameters) are used, and information criteria, such as AIC or BIC, are used to quantify the tradeoff between the performance and diagnostic accuracy of the panel and the number of DETERMINANTS used. The position of the individual DETERMINANT on a forward or backwards selected panel can be closely related to its provision of incremental information content for the algorithm, so the order of contribution is highly dependent on the other constituent DETERMINANTS in the panel.

Construction of Clinical Algorithms

Any formula may be used to combine DETERMINANT results into indices useful in the practice of the invention. As indicated above, and without limitation, such indices may indicate, among the various other indications, the probability, likelihood, absolute or relative risk, time to or rate of conversion from one to another disease states, or make predictions of future biomarker measurements of metastatic disease. This may be for a specific time period or horizon, or for remaining lifetime risk, or simply be provided as an index relative to another reference subject population.

Although various preferred formula are described here, several other model and formula types beyond those mentioned herein and in the definitions above are well known to one skilled in the art. The actual model type or formula used may itself be selected from the field of potential models based on the performance and diagnostic accuracy characteristics of its results in a training population. The specifics of the formula itself may commonly be derived from DETERMINANT results in the relevant training population. Amongst other uses, such formula may be intended to map the feature space derived from one or more DETERMINANT inputs to a set of subject classes (e.g. useful in predicting class membership of subjects as normal, at risk for having a metastatic event, having cancer), to derive an estimation of a probability function of risk using a Bayesian approach (e.g. the risk of cancer or a metastatic event), or to estimate the class-conditional probabilities, then use Bayes' rule to produce the class probability function as in the previous case.

Preferred formulas include the broad class of statistical classification algorithms, and in particular the use of discriminant analysis. The goal of discriminant analysis is to predict class membership from a previously identified set of features. In the case of linear discriminant analysis (LDA), the linear combination of features is identified that maximizes the separation among groups by some criteria. Features can be identified for LDA using an eigengene based approach with different thresholds (ELDA) or a stepping algorithm based on a multivariate analysis of variance (MANOVA). Forward, backward, and stepwise algorithms can be performed that minimize the probability of no separation based on the Hotelling-Lawley statistic.

Eigengene-based Linear Discriminant Analysis (ELDA) is a feature selection technique developed by Shen et al. (2006). The formula selects features (e.g. biomarkers) in a multivariate framework using a modified eigen analysis to identify features associated with the most important eigenvectors. “Important” is defined as those eigenvectors that explain the most variance in the differences among samples that are trying to be classified relative to some threshold.

A support vector machine (SVM) is a classification formula that attempts to find a hyperplane that separates two classes. This hyperplane contains support vectors, data points that are exactly the margin distance away from the hyperplane. In the likely event that no separating hyperplane exists in the current dimensions of the data, the dimensionality is expanded greatly by projecting the data into larger dimensions by taking non-linear functions of the original variables (Venables and Ripley, 2002). Although not required, filtering of features for SVM often improves prediction. Features (e.g., biomarkers) can be identified for a support vector machine using a non-parametric Kruskal-Wallis (KW) test to select the best univariate features. A random forest (RF, Breiman, 2001) or recursive partitioning (RPART, Breiman et al., 1984) can also be used separately or in combination to identify biomarker combinations that are most important. Both KW and RF require that a number of features be selected from the total. RPART creates a single classification tree using a subset of available biomarkers.

Other formula may be used in order to pre-process the results of individual DETERMINANT measurement into more valuable forms of information, prior to their presentation to the predictive formula. Most notably, normalization of biomarker results, using either common mathematical transformations such as logarithmic or logistic functions, as normal or other distribution positions, in reference to a population's mean values, etc. are all well known to those skilled in the art. Of particular interest are a set of normalizations based on Clinical Parameters such as age, gender, race, or sex, where specific formula are used solely on subjects within a class or continuously combining a Clinical Parameter as an input. In other cases, analyte-based biomarkers can be combined into calculated variables which are subsequently presented to a formula.

In addition to the individual parameter values of one subject potentially being normalized, an overall predictive formula for all subjects, or any known class of subjects, may itself be recalibrated or otherwise adjusted based on adjustment for a population's expected prevalence and mean biomarker parameter values, according to the technique outlined in D'Agostino et al, (2001) JAMA 286:180-187, or other similar normalization and recalibration techniques. Such epidemiological adjustment statistics may be captured, confirmed, improved and updated continuously through a registry of past data presented to the model, which may be machine readable or otherwise, or occasionally through the retrospective query of stored samples or reference to historical studies of such parameters and statistics. Additional examples that may be the subject of formula recalibration or other adjustments include statistics used in studies by Pepe, M. S. et al, 2004 on the limitations of odds ratios; Cook, N. R., 2007 relating to ROC curves. Finally, the numeric result of a classifier formula itself may be transformed post-processing by its reference to an actual clinical population and study results and observed endpoints, in order to calibrate to absolute risk and provide confidence intervals for varying numeric results of the classifier or risk formula. An example of this is the presentation of absolute risk, and confidence intervals for that risk, derivied using an actual clinical study, chosen with reference to the output of the recurrence score formula in the Oncotype Dx product of Genomic Health, Inc. (Redwood City, Calif.). A further modification is to adjust for smaller sub-populations of the study based on the output of the classifier or risk formula and defined and selected by their Clinical Parameters, such as age or sex.

Combination with Clinical Parameters and Traditional Laboratory Risk Factors

Any of the aforementioned Clinical Parameters may be used in the practice of the invention as aDETERMINANT input to a formula or as a pre-selection criteria defining a relevant population to be measured using a particular DETERMINANT panel and formula. As noted above, Clinical Parameters may also be useful in the biomarker normalization and pre-processing, or in DETERMINANT selection, panel construction, formula type selection and derivation, and formula result post-processing. A similar approach can be taken with the Traditional Laboratory Risk Factors, as either an input to a formula or as a pre-selection criterium.

Measurement of DETERMINANTS

The actual measurement of levels or amounts of the DETERMINANTS can be determined at the protein or nucleic acid level using any method known in the art. For example, at the nucleic acid level, Northern and Southern hybridization analysis, as well as ribonuclease protection assays using probes which specifically recognize one or more of these sequences can be used to determine gene expression. Alternatively, amounts of DETERMINANTS can be measured using reverse-transcription-based PCR assays (RT-PCR), e.g., using primers specific for the differentially expressed sequence of genes or by branch-chain RNA amplification and detection methods by Panomics, Inc. Amounts of DETERMINANTS can also be determined at the protein level, e.g., by measuring the levels of peptides encoded by the gene products described herein, or subcellular localization or activities thereof using technological platform such as for example AQUA. Such methods are well known in the art and include, e.g., immunoassays based on antibodies to proteins encoded by the genes, aptamers or molecular imprints. Any biological material can be used for the detection/quantification of the protein or its activity. Alternatively, a suitable method can be selected to determine the activity of proteins encoded by the marker genes according to the activity of each protein analyzed.

The DETERMINANT proteins, polypeptides, mutations, and polymorphisms thereof can be detected in any suitable manner, but is typically detected by contacting a sample from the subject with an antibody which binds the DETERMINANT protein, polypeptide, mutation, or polymorphism and then detecting the presence or absence of a reaction product. The antibody may be monoclonal, polyclonal, chimeric, or a fragment of the foregoing, as discussed in detail above, and the step of detecting the reaction product may be carried out with any suitable immunoassay. The sample from the subject is typically a biological fluid as described above, and may be the same sample of biological fluid used to conduct the method described above.

Immunoassays carried out in accordance with the present invention may be homogeneous assays or heterogeneous assays. In a homogeneous assay the immunological reaction usually involves the specific antibody (e.g., anti-DETERMINANT protein antibody), a labeled analyte, and the sample of interest. The signal arising from the label is modified, directly or indirectly, upon the binding of the antibody to the labeled analyte. Both the immunological reaction and detection of the extent thereof can be carried out in a homogeneous solution. Immunochemical labels which may be employed include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody can be immobilized on a support, such as a bead (such as protein A and protein G agarose beads), plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are oligonucleotides, immunoblotting, immunofluorescence methods, immunoprecipitation, chemiluminescence methods, electro chemiluminescence (ECL) or enzyme-linked immunoassays.

Those skilled in the art will be familiar with numerous specific immunoassay formats and variations thereof which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al. titled “Methods for Modulating Ligand-Receptor Interactions and their Application,” U.S. Pat. No. 4,659,678 to Forrest et al. titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogenous Specific Binding Assay Employing a Coenzyme as Label.”

Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies as described herein may likewise be conjugated to detectable labels or groups such as radiolabels (e.g., ³⁵S, ¹²⁵I, ¹³¹I) enzyme labels (e.g., horseradish peroxidase, alkaline phosphatase), and fluorescent labels (e.g., fluorescein, Alexa, green fluorescent protein, rhodamine) in accordance with known techniques.

Antibodies can also be useful for detecting post-translational modifications of DETERMINANT proteins, polypeptides, mutations, and polymorphisms, such as tyrosine phosphorylation, threonine phosphorylation, serine phosphorylation, glycosylation (e.g., O-GlcNAc). Such antibodies specifically detect the phosphorylated amino acids in a protein or proteins of interest, and can be used in immunoblotting, immunofluorescence, and ELISA assays described herein. These antibodies are well-known to those skilled in the art, and commercially available. Post-translational modifications can also be determined using metastable ions in reflector matrix-assisted laser desorption ionization-time of flight mass spectrometry (MALDI-TOF) (Wirth, U. et al. (2002) Proteomics 2(10): 1445-51).

For DETERMINANT proteins, polypeptides, mutations, and polymorphisms known to have enzymatic activity, the activities can be determined in vitro using enzyme assays known in the art. Such assays include, without limitation, kinase assays, phosphatase assays, reductase assays, among many others. Modulation of the kinetics of enzyme activities can be determined by measuring the rate constant K_{M} using known algorithms, such as the Hill plot, Michaelis-Menten equation, linear regression plots such as Lineweaver-Burk analysis, and Scatchard plot.

Using sequence information provided by the database entries for the DETERMINANT sequences, expression of the DETERMINANT sequences can be detected (if present) and measured using techniques well known to one of ordinary skill in the art. For example, sequences within the sequence database entries corresponding to DETERMINANT sequences, or within the sequences disclosed herein, can be used to construct probes for detecting DETERMINANT RNA sequences in, e.g., Northern blot hybridization analyses or methods which specifically, and, preferably, quantitatively amplify specific nucleic acid sequences. As another example, the sequences can be used to construct primers for specifically amplifying the DETERMINANT sequences in, e.g., amplification-based detection methods such as reverse-transcription based polymerase chain reaction (RT-PCR). When alterations in gene expression are associated with gene amplification, deletion, polymorphisms, and mutations, sequence comparisons in test and reference populations can be made by comparing relative amounts of the examined DNA sequences in the test and reference cell populations.

Expression of the genes disclosed herein can be measured at the RNA level using any method known in the art. For example, Northern hybridization analysis using probes which specifically recognize one or more of these sequences can be used to determine gene expression. Alternatively, expression can be measured using reverse-transcription-based PCR assays (RT-PCR), e.g., using primers specific for the differentially expressed sequences. RNA can also be quantified using, for example, other target amplification methods (e.g., TMA, SDA, NASBA), or signal amplification methods (e.g., bDNA), and the like.

Alternatively, DETERMINANT protein and nucleic acid metabolites can be measured. The term “metabolite” includes any chemical or biochemical product of a metabolic process, such as any compound produced by the processing, cleavage or consumption of a biological molecule (e.g., a protein, nucleic acid, carbohydrate, or lipid). Metabolites can be detected in a variety of ways known to one of skill in the art, including the refractive index spectroscopy (RI), ultra-violet spectroscopy (UV), fluorescence analysis, radiochemical analysis, near-infrared spectroscopy (near-IR), nuclear magnetic resonance spectroscopy (NMR), light scattering analysis (LS), mass spectrometry, pyrolysis mass spectrometry, nephelometry, dispersive Raman spectroscopy, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, NMR and IR detection. (See, WO 04/056456 and WO 04/088309, each of which are hereby incorporated by reference in their entireties) In this regard, other DETERMINANT analytes can be measured using the above-mentioned detection methods, or other methods known to the skilled artisan. For example, circulating calcium ions (Ca²⁺) can be detected in a sample using fluorescent dyes such as the Fluo series, Fura-2A, Rhod-2, among others. Other DETERMINANT metabolites can be similarly detected using reagents that are specifically designed or tailored to detect such metabolites.

Kits

The invention also includes a DETERMINANT-detection reagent, e.g., nucleic acids that specifically identify one or more DETERMINANT nucleic acids by having homologous nucleic acid sequences, such as oligonucleotide sequences, complementary to a portion of the DETERMINANT nucleic acids or antibodies to proteins encoded by the DETERMINANT nucleic acids packaged together in the form of a kit. The oligonucleotides can be fragments of the DETERMINANT genes. For example the oligonucleotides can be 200, 150, 100, 50, 25, 10 or less nucleotides in length. The kit may contain in separate containers a nucleic acid or antibody (either already bound to a solid matrix or packaged separately with reagents for binding them to the matrix), control formulations (positive and/or negative), and/or a detectable label such as fluorescein, green fluorescent protein, rhodamine, cyanine dyes, Alexa dyes, luciferase, radiolabels, among others. Instructions (e.g., written, tape, VCR, CD-ROM, etc.) for carrying out the assay may be included in the kit. The assay may for example be in the form of a Northern hybridization or a sandwich ELISA as known in the art.

For example, DETERMINANT detection reagents can be immobilized on a solid matrix such as a porous strip to form at least one DETERMINANT detection site. The measurement or detection region of the porous strip may include a plurality of sites containing a nucleic acid. A test strip may also contain sites for negative and/or positive controls. Alternatively, control sites can be located on a separate strip from the test strip. Optionally, the different detection sites may contain different amounts of immobilized nucleic acids, e.g., a higher amount in the first detection site and lesser amounts in subsequent sites. Upon the addition of test sample, the number of sites displaying a detectable signal provides a quantitative indication of the amount of DETERMINANTS present in the sample. The detection sites may be configured in any suitably detectable shape and are typically in the shape of a bar or dot spanning the width of a test strip.

Alternatively, the kit contains a nucleic acid substrate array comprising one or more nucleic acid sequences. The nucleic acids on the array specifically identify one or more nucleic acid sequences represented by DETERMINANTS 1-360. In various embodiments, the expression of 2, 3, 4, 5, 6, 7, 8, 9, 10, 15, 20, 25, 40, 50, 100, 125, 150, 175, 200, 250, 275 or more of the sequences represented by DETERMINANTS 1-360 can be identified by virtue of binding to the array. The substrate array can be on, e.g., a solid substrate, e.g., a “chip” as described in U.S. Pat. No. 5,744,305. Alternatively, the substrate array can be a solution array, e.g., xMAP (Luminex, Austin, Tex.), Cyvera (Illumina, San Diego, Calif.), CellCard (Vitra Bioscience, Mountain View, Calif.) and Quantum Dots' Mosaic (Invitrogen, Carlsbad, Calif.).

Suitable sources for antibodies for the detection of DETERMINANTS include commercially available sources such as, for example, Abazyme, Abnova, Affinity Biologicals, AntibodyShop, Biogenesis, Biosense Laboratories, Calbiochem, Cell Sciences, Chemicon International, Chemokine, Clontech, Cytolab, DAKO, Diagnostic BioSystems, eBioscience, Endocrine Technologies, Enzo Biochem, Eurogentec, Fusion Antibodies, Genesis Biotech, GloboZymes, Haematologic Technologies, Immunodetect, Immunodiagnostik, Immunometrics, Immuno star, Immunovision, Biogenex, Invitrogen, Jackson ImmunoResearch Laboratory, KMI Diagnostics, Koma Biotech, LabFrontier Life Science Institute, Lee Laboratories, Lifescreen, Maine Biotechnology Services, Mediclone, MicroPharm Ltd., ModiQuest, Molecular Innovations, Molecular Probes, Neoclone, Neuromics, New England Biolabs, Novocastra, Novus Biologicals, Oncogene Research Products, Orbigen, Oxford Biotechnology, Panvera, PerkinElmer Life Sciences, Pharmingen, Phoenix Pharmaceuticals, Pierce Chemical Company, Polymun Scientific, Polysiences, Inc., Promega Corporation, Proteogenix, Protos Immunoresearch, QED Biosciences, Inc., R&D Systems, Repligen, Research Diagnostics, Roboscreen, Santa Cruz Biotechnology, Seikagaku America, Serological Corporation, Serotec, SigmaAldrich, StemCell Technologies, Synaptic Systems GmbH, Technopharm, Terra Nova Biotechnology, TiterMax, Trillium Diagnostics, Upstate Biotechnology, US Biological, Vector Laboratories, Wako Pure Chemical Industries, and Zeptometrix. However, the skilled artisan can routinely make antibodies, nucleic acid probes, e.g., oligonucleotides, aptamers, siRNAs, antisense oligonucleotides, against any of the DETERMINANTS in Table 1.

### EXAMPLES

### Example 1

### General Methods

Transgenic Mice and Primary Tumors

The reverse tetracycline transactivator, Tet promoter and the tyrosinase enhancer/promoter transgene were used as described (Ganss, Montoliu et al. 1994; Chin, Pomerantz et al. 1997; Chin, Tam et al. 1999). Mouse c-Met cDNA (a gift from George F Vande-Woude, Grand Rapids, Mich.) was cloned under the control of a Tet promoter similar to as described (Chin, nature 1999). Multiple transgene founder lines were generated at the expected frequency. The well-defined activator line (Tyr/rtTA, line 37-Chin, Nature 1999) and three independent reporter lines (Met15, Met28 and Met40) were used for these studies.

To activate transgene expression in vivo, MET transgenic mice were fed doxycycline in drinking water (2 ug/ml in sucrose water) at weaning age and observed for spontaneous tumor development. A subset of animals (3-wks) were anesthetized intraperitoneally with avertin (0.5 g/kg body weight) and wounded on the back with 20-mm oblong wounds followed by suturing Animals were observed biweekly for development of tumors or appearance of ill health. Premoribund animals or animals with significant tumor burdens were sacrificed, followed by detailed autopsies. Tumor specimens were fixed in 10% formalin and embedded in paraffin for histological analysis as previously described (Chin, L. et al Genes and Dev, 1997). In cases where sufficient specimens were available, primary tumors were flash-frozen for subsequent analyses and cell lines were generated.

Cell culture. Melanoma cell lines were derived from mouse tumors by digestion with collagenase+Hyaluronidase (2 mg/ml; Sigma) for 2 hours followed by cultivation with RPMI 1640 media (Gibco BRL) containing 10% FBS and 1% penicillin/streptomycin. Melanocyte cultures were generated from newborn mouse epidermis as described¹⁰ and maintained in RPMI 1640 containing 5% FBS, 1% penicillin/streptomycin, 200 pM cholera toxin, 200 nM 12-Otetradecanoylphorbol-13-acetate (TPA). Transgenic c-Met expression was induced in cultured cells by the addition of doxycycline at 2 ug/ml. M3 BRAF melanocytes, HMEL468 primed melanocytes, WM3211 and WM115 were maintained in RPMI 1640 containing 10% FBS, 1% penicillin/streptomycin. HMEL468 identifies a subclone of PMEL/hTERT/CDK4(R24C)/p53DD/BRAF^{V600E} cells as described in Garraway et al¹¹.

Histological analysis and immunohistochemical staining. Mice were sacrificed according to institute guidelines and organs were fixed in 10% buffered formalin and paraffin embedded. Tissue sections were stained with H&E to enable classification of the lesions and detection of tumor metastasis. For detection of c-Met protein and determining its activation state, tumor samples were immunostained with total c-Met and phospho c-Met (Tyr1349) antibodies from Cell Signaling Technology. Tumors were immunostained with S100 antibody from Sigma.

Gene expression by RT-PCR and Real-time Quantitative PCR. For analyses of gene expression, total RNA was isolated from primary cutaneous melanomas or from cultured cells using Trizol (Gibco BRL) according to manufacturer's protocol. Total RNA was treated with RQ1 DNAse (Promega) and 1 ug total RNA was used for reverse transcription reaction using Superscript II polymerase (Invitrogen) primed with oligo(dT). Coding regions were amplified by PCR or quantitative real time PCR using SYBR Green (Applied Biosystems) on an Mx3000P real-time PCR system (Stratagene). Ribosomal protein R15 was used as an internal expression control. Primer sequences are as follows: c-Met: 5′-TCTGTTGCCATCCCAAGACAACATTGATGG, 5′-AAATCTCTGGAGGAGGTTGG; HGF: 5′-CAAGGCCAAGGAGAAGGTTA, 5′-TTTGAAGTTCTCGGGAGTGA; Tyr: 5′-CCAGAAGCCAATGCACCTAT, 5′-AGCAATAACAGCTCCCACCA; TRP1: 5′-ATTCTGGCCTCCAGTTACCA, 5′-GGCTTCATTCTTGGTGCTTC; DCT: 5′-AACAACCCTTCCACAGATGC, 5′-TCTCCATTAAGGGCGCATAG; R15: 5′-CTTCCGCAAGTTCACCTACC, reverse-TACTTGAGGGGGATGAATCG. SMAD3 primers were from Superarray.

Gene Expression Profiling and Data Analyses. Met-driven and HRas-driven mouse tumor RNAs were extracted as described above, labeled and hybridized to Affymetrix GeneChip Mouse Genome 430 2.0 Arrays by the Dana-Farber Cancer Institute Microarray Core Facility according to the manufacturer's protocol. Expression data was processed using the R/bioconductor package (www.bioconductor.org). Analysis was performed as described¹². Briefly, the background correction method was MAS (v4.5), normalization method was constant, PM adjustment method was MAS (v5), expression value summary method was medianpolish (RMA). P/M/A call method was MASS. Probe sets with at least 2 present calls among all 12 tumor samples (16,434 probe sets) were selected for further differential expression analyses between six iMet tumors versus six iHRas tumors. Significance Analysis of Microarray (SAM 2.0; http://www-stat.stanford.edu/˜tibs/SAM/) was used for differential expression analysis¹³. Two class unpaired sample analysis was performed, followed by filtering for minimum 2 fold change and delta value adjustment so that the false discovery rate would be less than 0.05. The HOXA1-induced transcription analysis was conducted by SAM as described above using RNAs extracted from cells (HMEL468, WM115, WM3211) transduced with either GFP or HOXA1, followed by hybridization of labeled cDNA onto Affymetrix GeneChip Human Genome U133Plus2.0 by the Dana-Farber Cancer Institute Microarray Core Facility according to the manufacturer's protocol. The Ingenuity Pathways Analysis program (http://www.ingenuity.com/index.html) was used to further analyze the cellular functions and pathways that were significantly regulated in metastatic melanoma.

Comparison of Mouse Gene Expression and Human Array CGH Data. Nonredundant, differentially expressed probe sets obtained from the expression analysis of mouse tumors (described above) were mapped to human orthologs that showed copy number aberrations in human metastatic melanoma identified by array-CGH (GEO Accession #GSE7606). Homologene database (NCBI) was used to identify orthologous human genes for those differentially expressed in iMet vs. iRas tumors. Genes up-regulated or down-regulated in iMet tumors (versus iRas tumors) and amplified or deleted in human metastases, respectively, were selected.

Unsupervised clustering and Kaplan-Meiers survival analysis. Expression profiles of the metastatic determinants were used to cluster 295 breast tumors¹⁴;¹⁵ into two groups by k-means clustering using R (http://www.r-project.org/). Kaplan-Meier survival analyses for the two clustered group were carried out using survival package in R, and P-values were calculated using survival statistical package in R.

DNA constructs and low-complexity library. pRetrosuperSmad3 and p3TPLux were from Addgene (#15726 and 11767, respectively). For the low complexity cDNA library, 230 cDNAs representing 199 genes were obtained from the ORFeome collection (Dana-Farber Cancer Institute) and transferred in high-throughput to pLenti6/V5 DEST (Invitrogen) via Gateway recombination following the manufacture's suggestions. Candidate cDNAs scoring in the invasion screen were sequence and expression verified using the V5 epitope, and homogenous clone preparations were used for all invasion validation studies.

96-well viral production, transduction and transwell invasion assays. Approximately 3×10⁴ 293T cells were seeded in 100 ul per each well in 96-well flat bottom plates 24 hrs prior to transfection (˜90% confluent) in DMEM+10% FBS (antibiotic). For each well transfection, 150 ng viral backbone and 110 ng lentiviral packaging vectors were diluted to 15 ul using Opti-MEM (Invitrogen). The resulting vector mix was combined with 15 ul Opti-MEM containing 0.6 ul Liptofectamine2000 (Invitrogen), incubated RT for 20 min and added to the 100 ul media covering the 293T cells. The media was replaced with DMEM+10% FBS+P/S approximately 10 hrs post-transfection, and 4 viral supernatant collections were taken starting at 36 hrs post transfection and combined. 150 ul viral supernatant containing 8 ug/ml polybrene was added to target cells (HMEL468) that were seeded into 96-well flat bottom plates 24 hrs prior to infection (70-80% confluent). Cells were infected twice and allowed to recover in RPMI+10% FBS+P/S for 24 hours following the second infection, after which cells were trypsized and applied to 96-well tumor invasion plated (BD Bioscience) following the manufacture's recommendations. Invaded cells were detected with in vivo labeling using 4 uM Calcein AM (BD Bioscience) and measured by fluorescence at 494/517 nm (Abs/Em). Positive-scoring candidates were identified as those scoring 2× standard deviations from the vector control.

Transwell invasion assays. Standard 24-well invasion chambers (BD Biosciences) were utilized to assess invasiveness following the manufacture's suggestions. Briefly, cells were trypsinized, rinsed twice with PBS, resuspended in serum-free RPMI 1640 media, and seeded at 7.5×10⁴ cells/well for HMEL468, 2.0×10⁴ for WM3211 and 5.0×10⁴ for WM115. Chambers were seeded in triplicate or quadruplicate and placed in 10% serum-containing media as a chemo-attractant as well as in cell culture plates in duplicate as input controls. Following 22 hrs incubation, chambers were fixed in 10% formalin, stained with crystal violet for manual counting or by pixel quantitation with Adobe Photoshop (Adobe). Data was normalized to input cells to control for differences in cell number (loading control). For assessment of SMAD3 knock-down on HOXA1-medited invasion, a validated shRNA construct targeting SMAD3 (pSUPER-shSMAD3), and virus was generated using standard retrovirus production protocols. Control cells were transduced with non-targeting shRNA (pSUPER-shNT) in parallel for invasion comparison.

Xenograft and tail vein injection studies. HMEL468 cells were stably transduced with either GFP or HOXA1 virus. For xenograft studies, cells were implanted in bi-flanks of CB-17-scid (C.B-Igh-1b/IcrTac-Prkdcscid; Taconic) mice at 1×10⁶ cells/site subcutaneously. To assess lung seeding capability, 5.0×10⁵ cells were injected into the tail vein of CB-17-scid mice. All animals were monitored for tumor development, followed by necropsy and tumor histological analysis.

TGFβ reporter assay. Cells were seeded at 2×10⁵ cells per well in triplicate in 6 well plates 24 hours before transfection with the p3TPLux reporter (1 ug per well) and control reporter (Renilla, 20 ng per well). Following 24 hrs of incubation, cells were treated for 24 hours with TGFβ (20 ng/ml, R&D Systems) and were subjected to luciferase analysis (Promega) following manufacture's protocol using a Lumat LB9507 Luminometer to access reporter activation as indicated by the firefly/Renilla ratio. p-values were calculated using two-tailed T test.

Immunoblotting analysis. Cells were treated as indicated with 20 ng/ml TGFb (R&D Systems), followed by washing 2× in PBS and lysis using RIPA buffer (150 mM NaCl, 50 mM Tris-HCl, pH 7.5, 500 μM EDTA, 100 μM EGTA, 1.0% Triton X-100, and 1% sodium deoxycholate) containing 1 mM PMSF, 1× Protease Inhibitor Cocktail (Sigma) and 1× Phosphatase inhibitor (Calbiochem). Following 30 min incubation in lysis buffer at 4° C., whole cell extracts were separated were cleared by centrifugation at 10 k 10 min 4° C., then protein concentrations were determined by DC Protein Assay (BioRad). Proteins were visualized by separation on NuPAGE 4-12% Bis-Tris gels (Invitrogen), blotted onto PVDF (Millipore, Billerica, Mass.) blocked with %5 milk in PBS+Tween-20, then incubated with the indicated antibodies. The following antibodies were used for immunoblotting: pSmad3 and total Smad3 (Cell Signaling Technology), alpha-tubulin (Sigma), V5 (Invitrogen), phospho-FAK (pY397; Invitrogen).

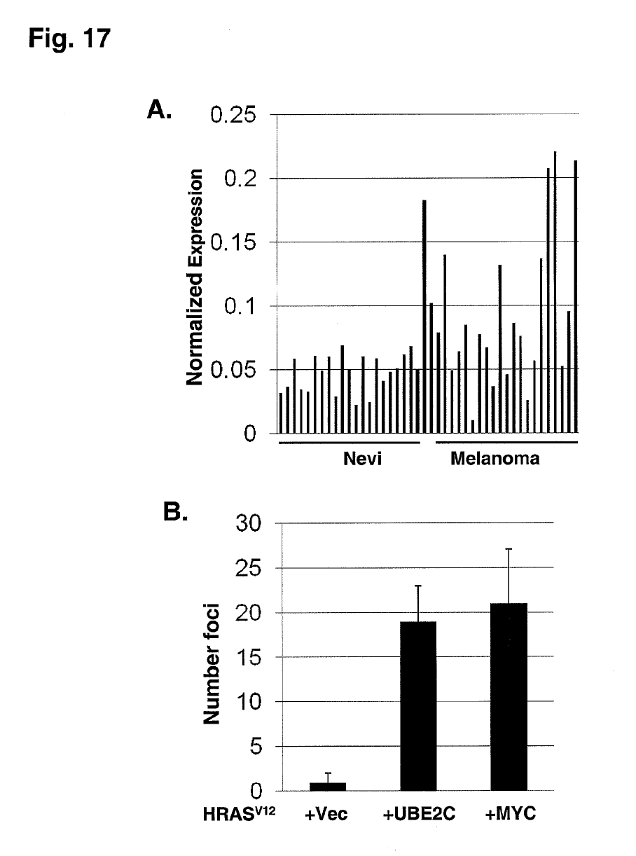

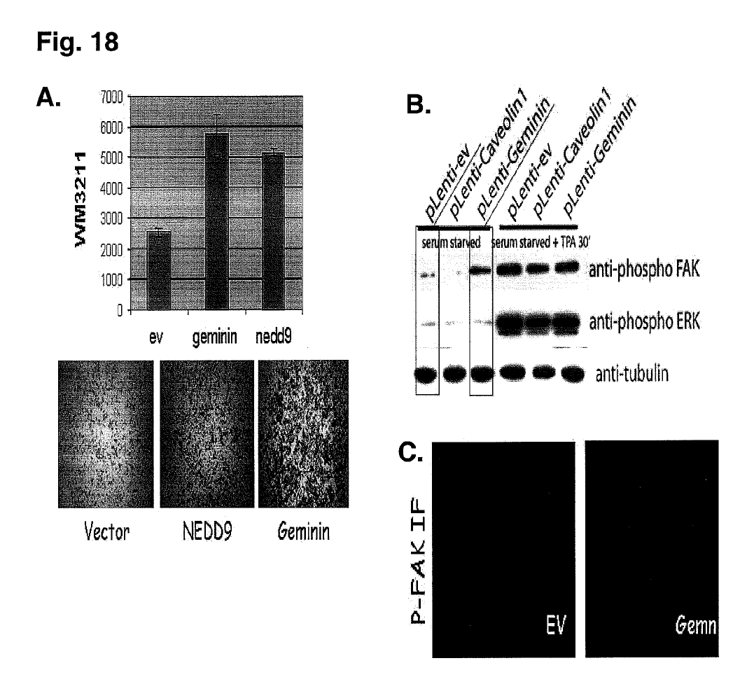

RNA-based expression assay by Panomics technology As an alternative to protein-based expression analysis, QuantiGene Plex technology (Panomics) was also utilized o assess the RNA expression of PDs. The QuantiGene platform is based on the branched DNA technology, a sandwich nucleic acid hybridization assay that provides a unique approach for RNA detection and quantification by amplifying the reporter signal rather than the sequence (Flagella, M., Bui, S., Zheng, Z., Nguyen, C. T., Zhang, A., Pastor, L., Ma, Y., Yang, W., Crawford, K. L., McMaster, G. K., et al. (2006) A multiplex branched DNA assay for parallel quantitative gene expression profiling. Anal Biochem 352, 50-60). This technology can reliably measure quantitatively RNA expression in fresh, frozen or formalin-fixed, paraffin-embedded (FFPE) tissue homogenates (Knudsen, B. S., Allen, A. N., McLerran, D. F., Vessella, R. L., Karademos, J., Davies, J. E., Maqsodi, B., McMaster, G. K., and Kristal, A. R. (2008) Evaluation of the branched-chain DNA assay for measurement of RNA in formalin-fixed tissues. J Mol Diagn 10, 169-176.) As shown in FIG. 17A, a feasibility pilot has shown that we can reliably measure the RNA expression of UBE2C in 21 spitz nevi and 22 malignant melanoma specimens that are in FFPE blocks. Analysis of each gene achieved excellent reproducibility with Coefficient of Variation (CV) values in the 8-9% range, thus meeting maximum quality control standards. This methodology thus provides an ideal alternative for us to glean first insight into expression pattern of a candidate of interest without available antibody. Of note, the QuantiGene Plex analysis of UBE2C corroborated results indicating oncogenic activity of UBE2C. Specifically, using the classical co-transformation assay we show that UBE2C cooperated with activated HRAS^{V12} to increase transformed focus formation in Ink4a/Arf-deficient primary mouse embryonic fibroblasts (FIG. 17B)

Automated Quantitative Analysis (AQUA®) allows exact measurement of protein concentration within subcellular compartments, as described in detail elsewhere [Camp, R. L., Chung, G. G., & Rimm, D. L., Automated subcellular localization and quantification of protein expression in tissue microarrays. Nat Med 8 (11), 1323-1327 (2002)]. In brief, a series of high resolution monochromatic images were captured by the PM-2000 microscope. For each histospot, in- and out-of-focus images were obtained using the signal from the DAPI, cytokeratin and primary antibody-specific signals. Tumor was distinguished from stromal and non-stromal elements by creating al tumor “mask” from the cytokeratin and S100 signal. This created a binary mask (each pixel being either “on” or “off”) on the basis of an intensity threshold set by visual inspection of histospots. AQUA® score of the protein of interest in each subcellular compartment was calculated by dividing the signal intensity (scored on a scale from 0-255) by the area of the specific compartment. Specimens with less that 5% tumor area per spot were not included in automated quantitative analysis for not being representative of the corresponding tumor specimen.

### Example 2

### Identification of a Phenotype-Driven Evolutionarily Conserved Metastasis Signature

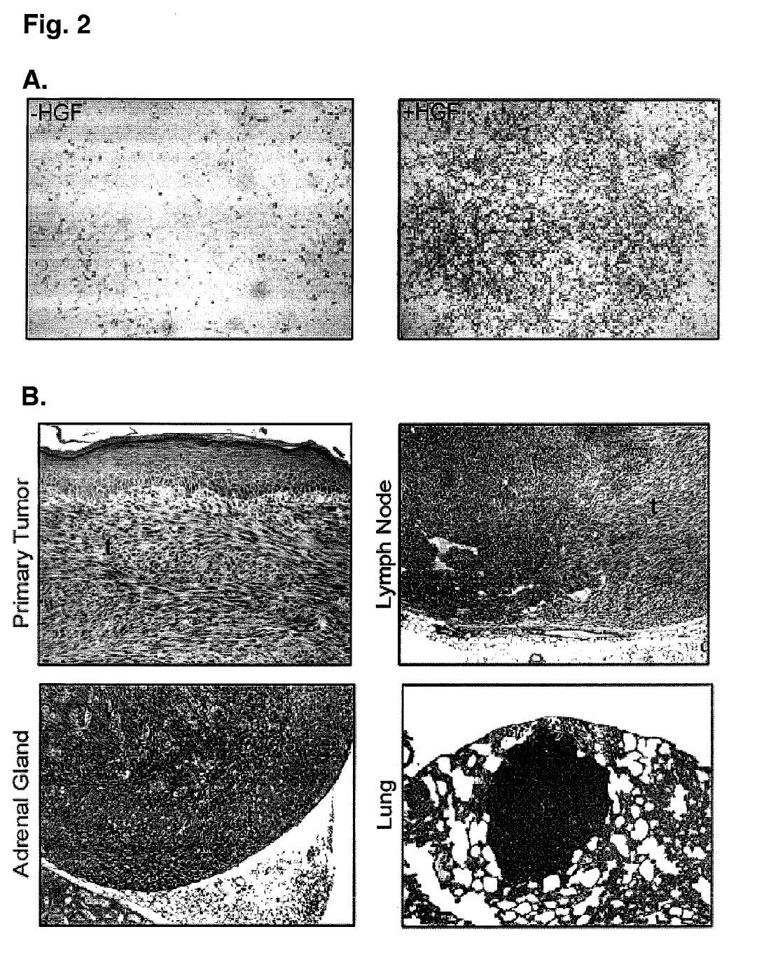

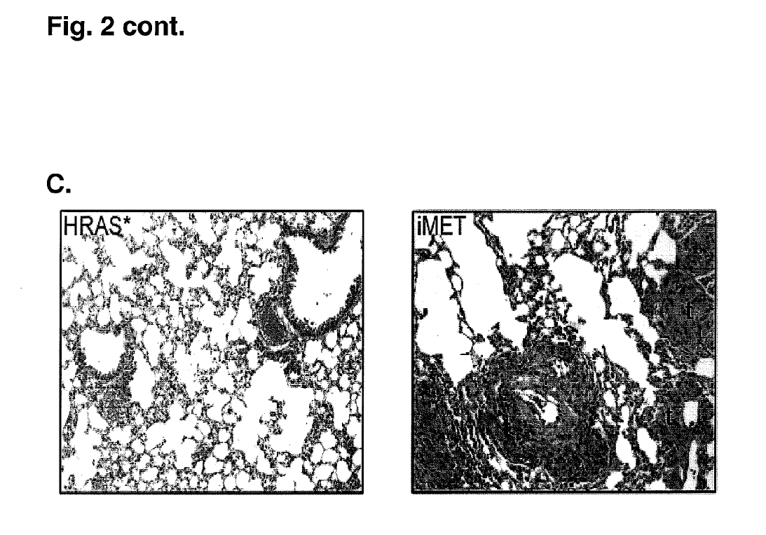

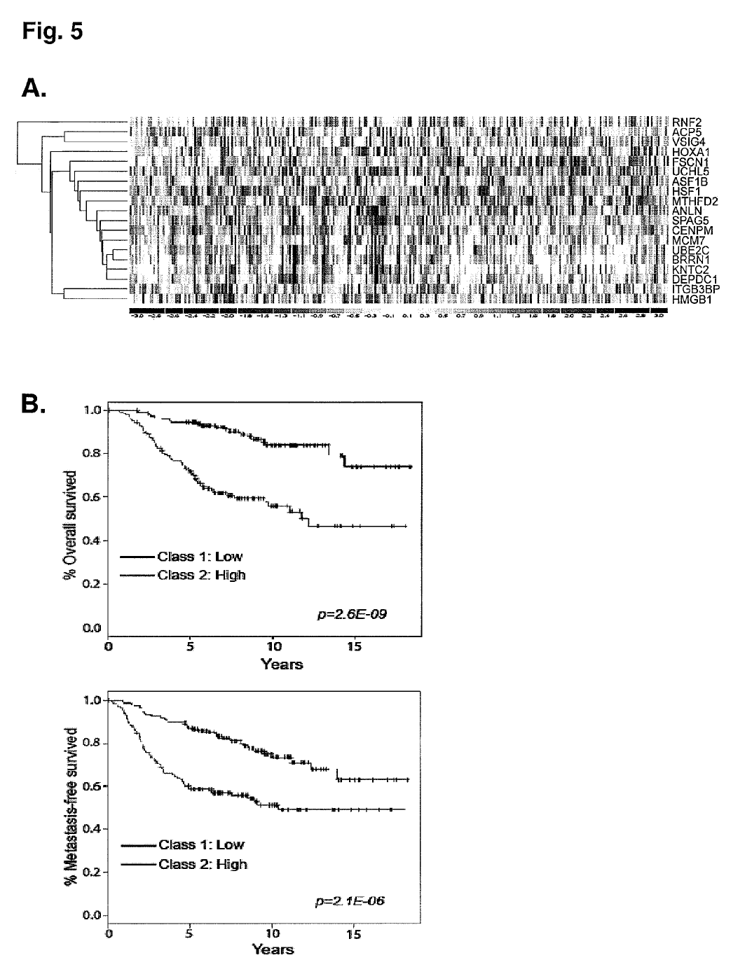

Genetically-engineered mouse (GEM) models of melanoma with very different metastatic potentials were used here as one biological system to mitigate confounding uncertainties inherent in the analysis of human cancers that include, among others, variables relating to documentation of micro- or macro-metastasis and duration of follow-up. The two mouse melanoma models utilized were (i) a newly developed Met-driven GEM model comprised of tyrosinase-driven rtTA and tet-Met transgenes on the Ink4a/Arf null background (Tyr-rtTA;Tet-Met;Ink4a/Arf^{-/-}, hereafter “iMet”) and (ii) the previously described HRAS^{V12G}-driven mouse melanoma model (Tyr-rtTA;Tet-HRAS^{V12G};Ink4a/Arf^{-/-}, hereafter “iHRAS*”)¹². Phenotypic characterization has shown that 75% of the iMet mice develop melanoma at sites of biopsy with an average latency of 12 weeks. These tumors are melanocyte marker positive, show phosphor-activated Met receptor and HGF expression (FIG. 5A-E); additionally, derivative iMet melanoma cells show robust invasion activity in transwell chamber invasion assays in response to recombinant HGF (FIG. 2A). Consistent with activation of HGF-MET signaling in advanced metastatic melanoma in human¹³, iMet melanomas in de novo transgenic animals uniformly metastasize to the lymph nodes in addition to occasional dissemination to the adrenal glands and lung parenchyma, each common sites of metastatic seeding in human melanoma and (FIG. 2B). This highly penetrant metastatic phenotype is in sharp contrast with the iHRAS* melanoma model which is characterized by non-metastatic primary cutaneous melanomas^{12;14}. This contrasting metastatic potential was reinforced by demonstration that iMet, but not iHRAS*, cell lines derived from primary melanomas were capable of seeding the lung in tail-vein assays (FIG. 2C).

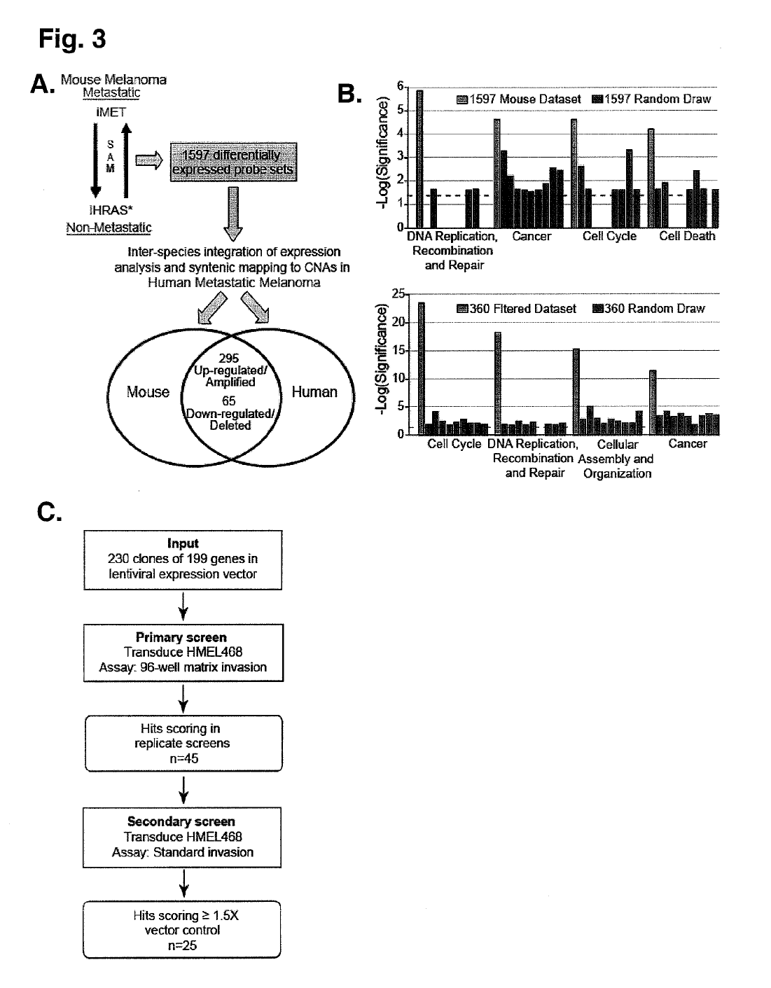

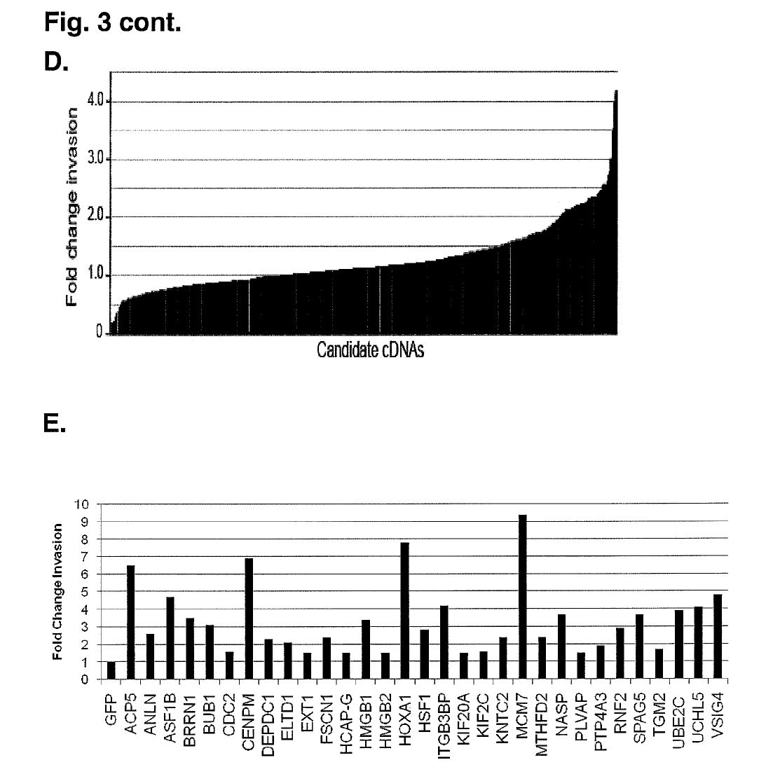

The clear-cut differences between iHRAS* and iMet metastatic propensity permitted the generation of a phenotype-driven primary tumor metastasis signature based on transcriptomic comparisons of primary cutaneous melanomas from the iHRAS* and iMet models. This mouse metastasis signature comprising of 1597 probe sets with ≧2-fold differential expression at a false discovery rate <0.05 was next interfaced with a large compendium of genes that (i) reside in copy number aberrations (CNAs) in human metastatic melanoma and/or (ii) exhibit differential expression between human primary and metastatic melanomas, yielding 295 up-regulated/amplified and 65 down-regulated/deleted genes (FIG. 3A; Table 4). To glean early insight into the types of biological activities conferred by these genes, we performed knowledge-based pathway analysis using Ingenuity Pathway Analysis (IPA) (Ingenuity Systems Inc., Redwood City, Calif.) to define which gene functions scored significantly by the 360 filtered gene list versus the larger 1597 murine metastasis signature. To assess the significance of the WA calls, we generated random draw lists of identical sizes for parallel analysis. As shown in FIG. 3B, we found that the murine metastasis expression signature showed some over-representation, relative to the random draw lists, of gene functions involved in DNA Replication and Recombination, Cancer, Cell Cycle and Cell Death. By comparison, the cross-species/cross-platform filtered list showed markedly stronger enrichment for these same functions in addition to emergence of a new functional network not apparent in the murine expression signature only, namely, ‘Cell Assembly and Organization’ (FIG. 3B). This comparison suggested that the triangulation of a phenotype-driven metastasis signature and cross-species comparison can serve to enrich for gene networks with strong links to processes of tumorigenesis and metastasis.

### Example 3

### Functional Genetic Screen for Metastasis Determinants

In particular, the strong enrichment of Cellular Assembly and Organization genes was encouraging given the relevance to cell movement and invasion which are obligate capabilities of a disseminating cancer cell. This observation motivated us to implement a low-complexity genetic screen for identification of genes driving invasion (FIG. 3C); furthermore, these screens focused exclusively on up-regulated genes given their possible therapeutic potential. Specifically, 230 available ORFs corresponding to 199 of the 295 unique up-regulated/amplified candidates (Table 5) were obtained from the human ORFeome (http://horfdb.dfci.harvard.edu/) and transferred to a lentiviral expression system for transduction into HMEL468, a TERT-immortalized primary human melanocyte line¹⁵. For the primary screen, we utilized a 96-well transwell invasion assay with fluorometric readout to measure the ability of candidate determinant genes to enhance migration and invasion of HMEL468 through matrigel which simulates extracellular matrix. As negative and positive controls, GFP and NEDD9¹⁶ lentivirus were used, respectively. The primary screen was repeated twice and 45 candidates reproducibly scoring two standard deviations from the GFP control were considered primary screen hits (FIG. 3C-D). A secondary validation screen of the 45 primary hits was then performed in triplicate using standard 24-well matrigel transwell invasion chambers, yielding 25 genes capable of at least 1.5-fold enhancement of invasion compared to the GFP control in HMEL468 melanocytes (FIG. 3E and Table 3). In addition, related genes or genes known to be in complex with one of these 25 determinants were also enlisted into functional assay, identifying an additional 6 determinants.

### Example 4

### Progression-Correlation of Expression in Human Primary and Metastatis Melanoma TMA

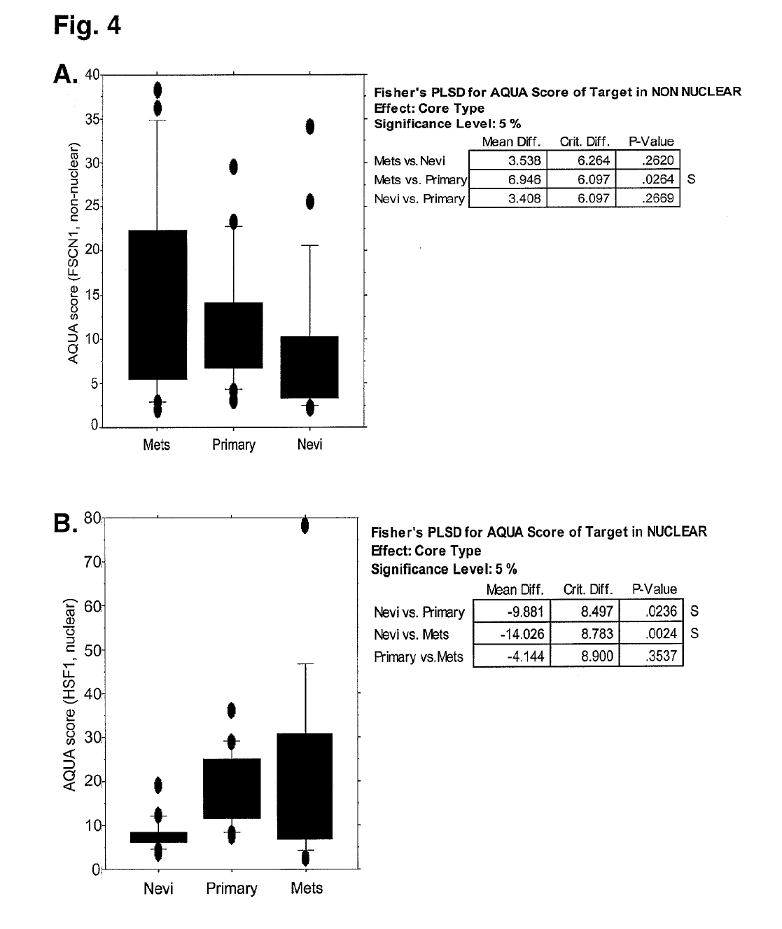

In effort to correlate expression of metastasis determinants with progression of malignant melanoma, we performed IHC analysis of tissue microarrays (TMAs) containing specimens of benign nevi, primary melanoma and melanoma metastases using commercially available antibodies against representative determinants by AQUA as described (Camp, R. L., Dolled-Filhart, M., King, B. L., and Rimm, D. L. (2003). Quantitative analysis of breast cancer tissue microarrays shows that both high and normal levels of HER2 expression are associated with poor outcome. Cancer Res 63, 1445-1448.). As summarized in Table 2 and representative data in FIG. 4A-B, with the exception of BRRN1, all other tested determinants (HSF1, MCM7, HOXA1, FSCN1, ACP5, UBE2C and KNTC2) exhibit significantly higher expression in primary of metastases versus benign nevi.

TABLE 2

| | Nevi vs. | Nevi vs. | Primary | | |
|---|---|---|---|---|---|
| Determinant | Primary | Met | vs. Met | Antibody | Expression Summary |
| HSF1 | 0.0236* | 0.0024* | 0.3537 | abnova; H00003297-A01 | Mets/Primary higher than |
| | | | | | Nevi |
| HOXA1 | <0.0001* | <0.0001* | 0.9017 | abnova; H00003198- | Mets/Primary higher than |
| | | | | B01P | Nevi |
| FASCIN | 0.2669 | 0.2621 | 0.0264* | santa cruz; sc21743 | Mets higher than |
| | | | | | Primary/Nevi |
| ACP5 | 0.2502 | 0.0014* | 0.0262* | Abcam; ab49507 | Mets higher than Primary, |
| | | | | | Primary trend higher than |
| | | | | | Nevi |
| UBE2C | 0.0046* | 0.7833 | 0.0162* | Abcam; 12290 | Interesting trends with |
| | | | | | Mets highest, Primary |
| | | | | | higher than Nevi |
| KNTC2 | 0.2248 | 0.3579 | 0.0338* | abnova; H00010403- | Mets higher than |
| | | | | M01 | Primary/Nevi |
| MCM7 | 0.0246* | 0.0025* | 0.3527 | abnova; H00004176- | Mets/Primary higher than |
| | | | | M01 | Nevi |
| BRRN1 | 0.0349* | 0.0607* | 0.8057 | Bethyl; A300-603A | Nevi higher than Primary |

Values indicate P-value test of indicated AQUA ® score comparison
*Significant by Fisher's test, 5%.

### Example 5

### Metastasis Determinants are Non-Lineage Specific and Prognostic

It is well established that genomic instability drives tumorigenesis, creating primary tumors comprised of heterogeneous subpopulations of cells with common and distinct genetic profiles. It thus stands to reason that, if a metastasis determinants-expressing sub-population within a primary tumor is endowed with a proliferative advantage and ultimately disseminates, the expression of the metastasis determinants would increase due to enriched representation in the more homogeneous derivative metastatic lesions. To assess for such progression-associated expression, the 25 determinants were examined in the large compendium of expression profiling data on Oncomine²⁴. In addition to seven determinants showing increased expression in metastatic relative to primary melanoma, all 25 exhibited a progression-correlated expression pattern in one or more non-melanoma solid tumors (Table 3), even though the majority of these 25 metastasis determinants have not been previously implicated in tumor progression. For instance, 9 determinants showed statistically significant increase in expression in higher grade gliomas. In prostate adenocarcinoma, 9 of the metastasis determinants exhibited significant increase in expression from primary to metastasis. Similarly, in lung, 5 exhibited correlation with increasing tumor grades. The most significant overlap was observed with breast adenocarcinoma, where 12 of the 25 metastasis determinants showed correlation with stages or grades of tumor progression.

Given the significant overlap in breast adenocarcinoma profile, we next made use of the published outcome-annotated transcriptome data in breast^{5;6} to explore the potential broader prognostic significance of these determinants. The breast cancer transcriptome dataset included probes for 19 of the 20 metastasis determinants, which were used as signature to stratify a cohort of 295 breast tumors by k-means unsupervised classification algorithm (FIG. 5A; Table 7). The resultant subgroups were found to have significant difference in overall survival (p=2.6⁻⁹) and metastasis-free survival (p=2.1⁻⁶) (FIG. 5B). Similar separation was obtained when classification was performed using hierarchical clustering (data not shown).

The robust prognostic potential in early-stage breast cancers and the broad pattern of progression-correlated expression in multiple non-melanoma cancer types indicate that these 25 metastasis determinants are not lineage-specific and likely driving core processes operating in diverse tumor types, although most of them have not been implicated in invasion or metastasis in the literature. Instead, many are annotated on Gene-Ontology as cell-cycle or proliferation genes with known roles in spindle checkpoint regulation or chromosome condensation. For example, several determinants (e.g. BRRN1, KNTC2, SPAG5, UBE2C, CENPM and MCM7) are known to regulate processes of DNA mitotic progression, mitotic spindle and DNA replication. On the other hand, BRRN1, KNTC2 and UBE2C are included in a 20-genes functional module enriched in a metastatic breast cancer signature associated with primary breast tumors that metastasized relative to primary tumors that do not²⁵. Similarly, MCM7 has been identified as a poor prognostic marker for multiple invasive cancers, including prostate cancer²⁶. Taken together, while it is yet unclear how these proteins contribute, directly or indirectly, to driving cell invasion and metastasis, we speculate that these mitotic checkpoint proteins may serve dual roles in controlling the cytoskeletal machinery for cell movement.

### Example 6

### Identification of Genes that Confer Anoikis Resistance

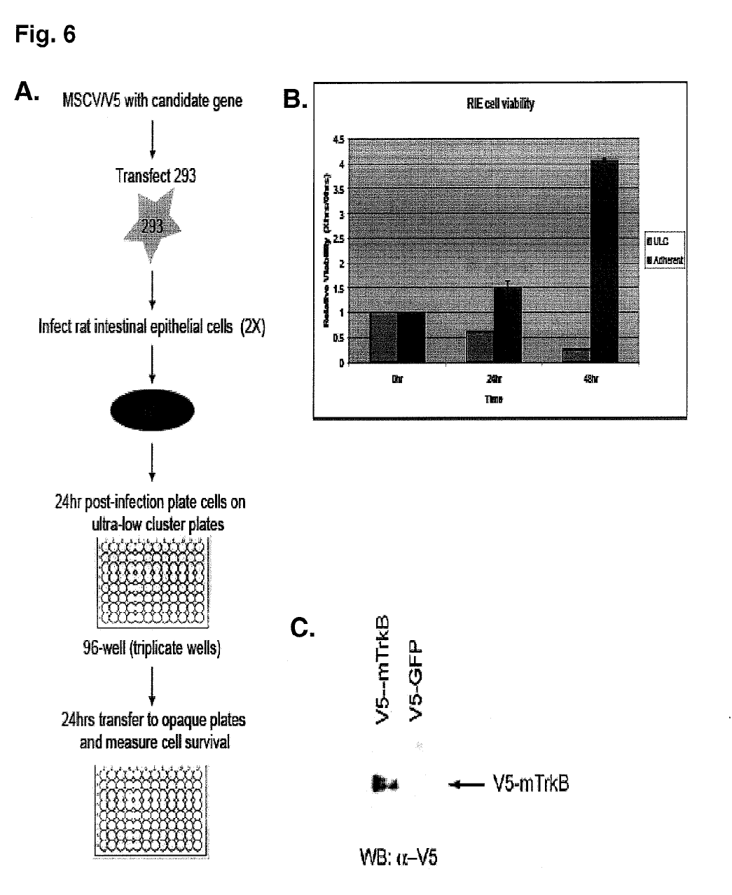

Metastasis is a complex, multi-step process (Gupta, G. P., and Massague, J. (2006) Cancer metastasis: building a framework. Cell 127, 679-69). In order for full metastasis to occur tumor cells must be able to proliferate at the primary tumor site, intravasate into the circulatory or lymphatic system, survive while in circulation, extravasate and form a secondary tumor. To accomplish this, circulating tumor cells must be able to overcome anoikis, or apoptosis induced by loss of matrix attachment (Simpson, C. D., Anyiwe, K., and Schimmer, A. D. (2008) Anoikis resistance and tumor metastasis. Cancer Lett 272, 177-185). In order to identify genes that confer anoikis resistance to anoikis sensitive cells, we optimized an in vitro screen for anoikis sensitivity (FIG. 6A). We hypothesized that cells seeded on a plate (ultra-low cluster) coated with a hydro-gel layer that prevented cell surface attachment would partially recapitulate in vitro the in vivo suspension of cells while in circulation.

In pilot studies, we screened a cohort of melanoma cell lines and found all, irrespective of melanoma stage (e.g. localized, invasive), anoikis resistant. Instead, we and others found rat intestinal epithelial (RIE) cells to have reduced survival upon loss of adherence (FIG. 6B) (Douma, S., Van Laar, T., Zevenhoven, J., Meuwissen, R., Van Garderen, E., and Peeper, D. S. (2004) Suppression of anoikis and induction of metastasis by the neurotrophic receptor TrkB. Nature 430, 1034-1039). RIE cells are immortalized but not transformed cell line. Cells undergoing anoikis initiate apoptotic pathways, while those that are viable upon loss of attachment demonstrate anoikis resistance. Therefore, we measured ATP generation, indicative of cellular metabolism, as a quantifiable and sensitive measure of cell viability.

Using the Gateway recombination system, 199 of the candidate ORFs identified through our cross-species oncogenomic analyses were cloned into the retroviral vector, MSCV/V5. As analyzed by Western blot, mTrkB and a randomized sampling of clones of varying cDNA size expressed in RIE, thereby demonstrating the functionality of our expression system (FIG. 6C and data not shown).

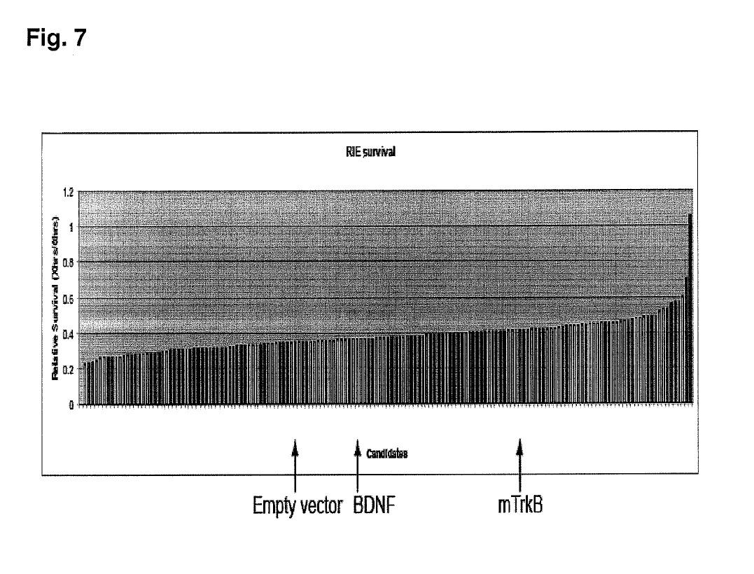

For the anoikis resistance screen, 293T cells were plated on 6-well plates and co-transfected with MSCV/V5 containing one ORF and the packaging vector, pCL-Eco (FIG. 6A). Cells were transfected with Lipofectamine 2000 (Invitrogen) and virus was harvested at multiple time points. RIE cells were plated on 6-well and 24 hr after plating were serially infected with 48 hr and 72 hr viral supernatant. RIE were harvested 24 hr after final infection and after generation of single-cell suspension, 7000 cells/well were plated in triplicate on 96-well ULC plates (time 0 hr). To determine baseline cell number, cells were lysed at 0 hr and ATP levels were measured (Cell Titer Glo, Promega). At 24 hr post-ULC plating, cells were lysed with Cell Titer Glo and lysate was transferred to 96-well opaque-welled luminometer plates for reading. In our analysis, ATP levels were compared at 24 hr relative to 0 hr thereby giving the fold change in ATP levels (FIG. 7).

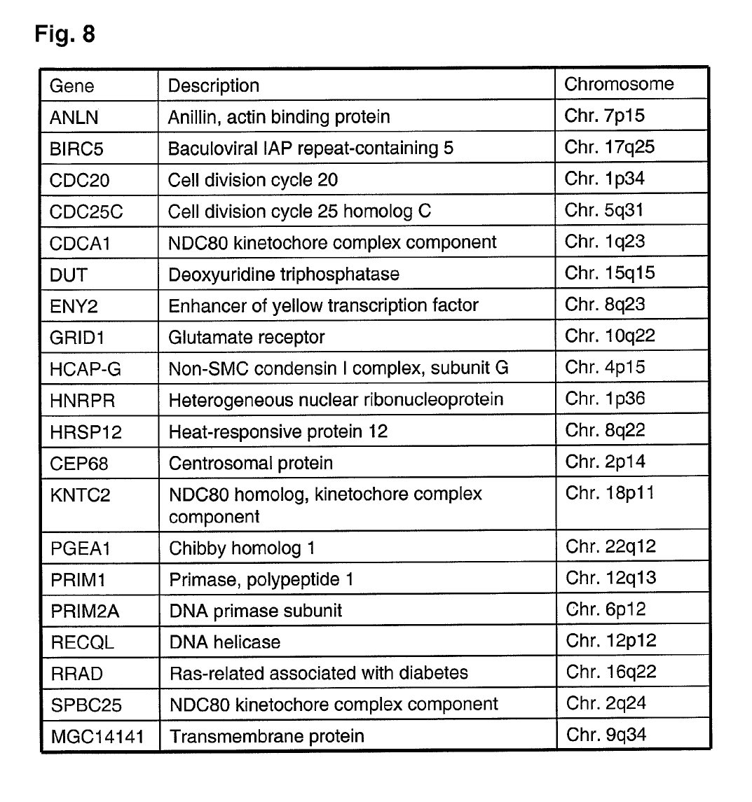

The neurotrophic receptor TrkB has been shown to confer anoikis resistance in vitro to anoikis sensitive cells and promote tumor formation and lung seeding in vivo (3). We have increased confidence in our screen since murine TrkB (mTrkB) and the human ligand to TrkB, BDNF, conferred anoikis resistance to RIE greater than vector alone (FIG. 7). In identical duplicate screens, an average of 21% of genes conferred greater than 1 standard deviation from the median of all candidate genes. Twenty genes gave greater than 2 standard deviations from the median in at least one pass of the screen (FIG. 8). Nine of these genes conferred greater than 1 standard deviation from the median in both screens, while seven genes of these nine gave greater than 2 standard deviations from the median in at least one pass of the screen (HNRPR, CDC20, PRIM2A, HRSP12, ENY2, MGC14141, RECQL). Interestingly, of the nine genes, STK3, PRIM2A, CDC20, RECQL, HNRPR, ENY2 and MGC14141 have shown greater expression in melanoma samples either in normal vs. melanoma or primary vs. metastases (Oncomine, GEO). In addition, all nine genes have shown increased expression in either breast, lung or brain tumors demonstrating that our priority list has validity in other cancer types as well (Oncomine).

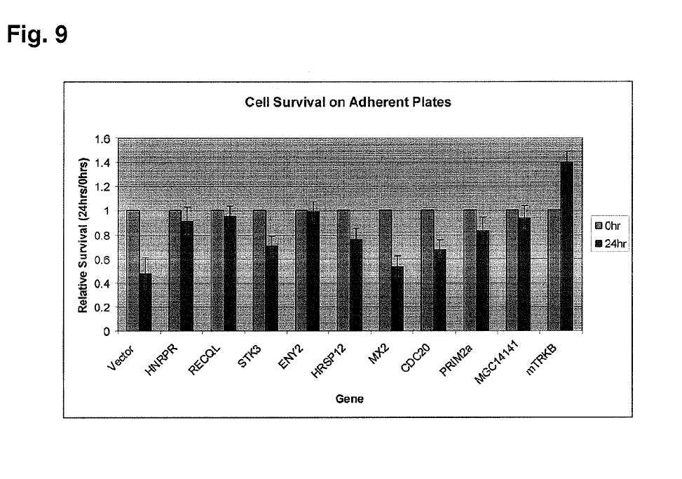

In order to confirm the increased viability of cells expressing our nine candidate genes in non-adherent conditions, we examined the retention of attachment capabilities after a period of loss of attachment. RIE expressing genes of interest were transferred to ULC plates and after 24 hrs all cells in suspension were transferred to adherent plates. Adherent cells were stained with crystal violet to quantify viable cells. As shown in FIG. 9, RIE cells had reduced ability to attach to adherent plates after being in suspension for 24 hr. However, all nine genes conferred increased ability of RIE to re-attach and remain viable after cells had been in suspension (FIG. 9). Such a capability would be a necessary characteristic of circulating tumor cells that were destined to colonize at a secondary site.

### Example 7

### Metastasis Determinants are Oncogenic

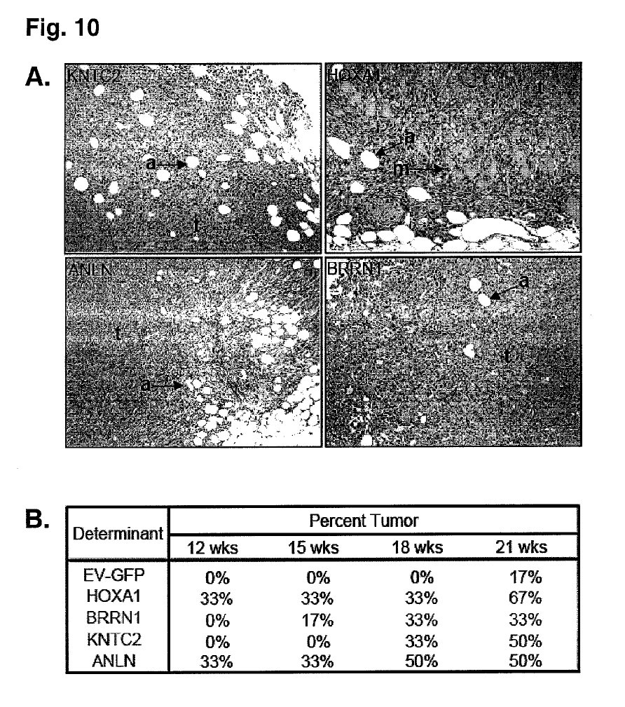

Since metastasis determinants are acquired early in the transformation process and pre-existing in primary tumors, it has been postulated that these metastasis genes might also be bona fide cancer genes that provide a proliferative advantage to the primary tumors^{2;22}. To address this, we asked whether these metastasis determinants could confer frank tumorigenicity to TERT-immortalized melanocytes, HMEL468. In addition to HOXA1, we also selected three other determinants for testing, namely ANLN, BRRN1 and KNTC2, since they are included on a 254-gene signature anti-correlated with metastasis-free survival in melanoma²³. Indeed, HOXA1-transduced HMEL468 developed large tumors (2 cm) with histopathological evidence of local invasion (FIG. 10A) with penetrance of 33% (n=2 of 6 subcutaneous transplanted sites) after 12 weeks, whereas vector-transduced controls did not develop any tumor through 21 weeks post injection (FIG. 10B). Similarly, ANLN, BRRN1 and KNTC2 transduced cells exhibited enhanced tumorigenicity relative to vector control (FIG. 3B). Taken together, we conclude that metastasis determinants are indeed bona fide oncogenes themselves which can also drive invasive behavior.

### Example 8

### In Vitro and In Vivo Validation Data for Hoxa1 and FSCN1

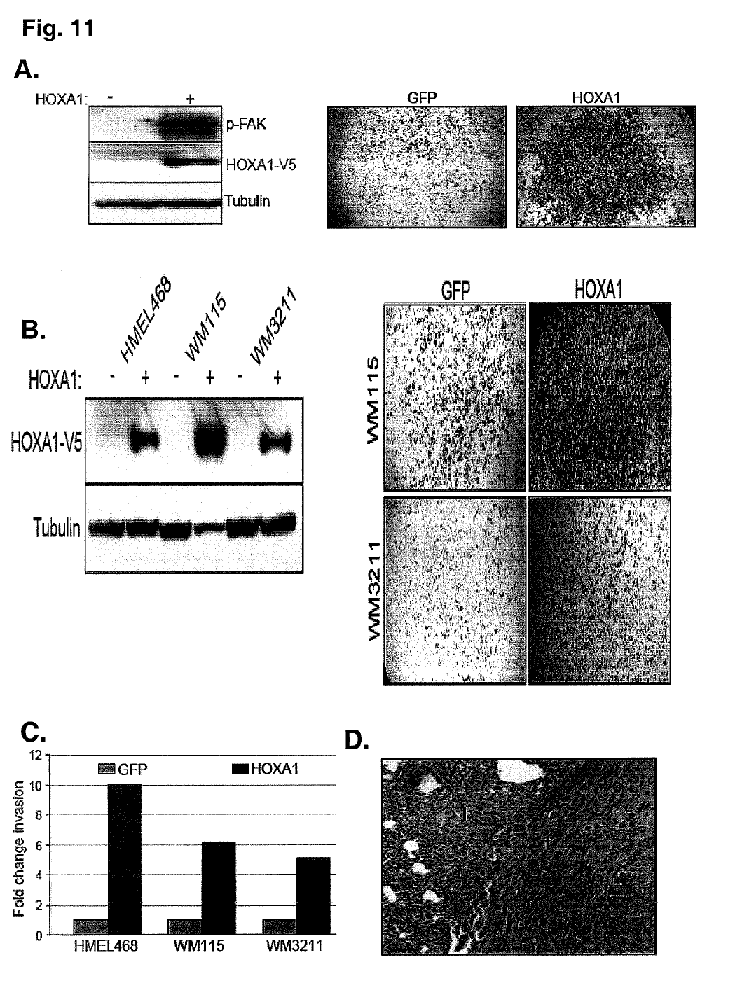

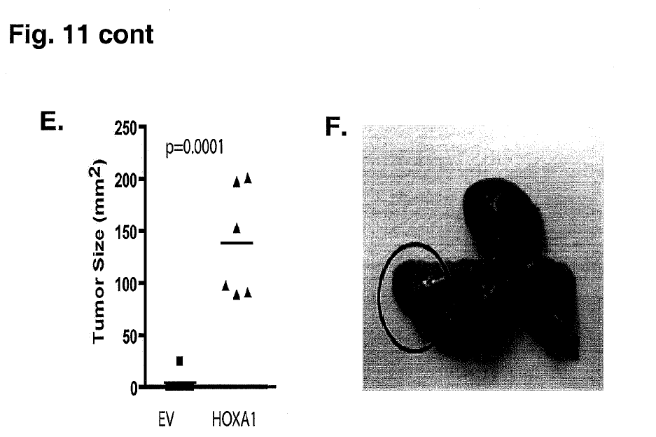

To further validate this integrated approach as a means of identifying metastasis determinants, we next conducted in-depth validation of the homeobox transcription factor, HOXA1. HOXA1 up-regulation has been reported in multiple cancers including breast, NSCLC and melanoma^{17;18;19}, although a role in invasion and metastasis has not been suggested. In over-expression studies, enforced HOXA1 elicited dramatically increased phosphorylation of focal adhesion kinase, FAK (FIG. 11A), a key signaling molecule in the regulation of growth factor and integrin-stimulated cell motility and invasion²⁰. Accordingly, HOXA1-overexpressing HMEL468 exhibited a 10-fold increase in invasion in vitro and acquired in vivo lung seeding capability (FIG. 11A, D). Importantly, this pro-invasion activity was not specific to the HMEL468 melanocyte cell line, as HOXA1 was able to similarly enhance invasion of WM115 and WM3211 human melanoma cells (FIG. 11B-C). Indeed, as summarized in Table 3, many of the determinants subjected to invasion assays in WM115 and WM3211 melanoma cells also showed pro-invasive activity beyond HMEL468 melanocytes. Additional validation assays testing the oncogenic and metastatic potential of HOXA1 using the weakly oncogenic melanoma cell line, WM115, indicate that HOXA1 over-expression markedly enhances tumor growth of xeno-transplanted cells in nude mice (FIG. 11E) consistent with data using other human and mouse cell lines. HOXA1 over-expression also lead to increased tumor growth of WM115 cells when implanted intradermally into the flanks of nude mice, and resulting primary tumors readily metastasized to the lungs following tumor development (FIG. 11F) whereas control (empty vector cells) do not form primary tumors.

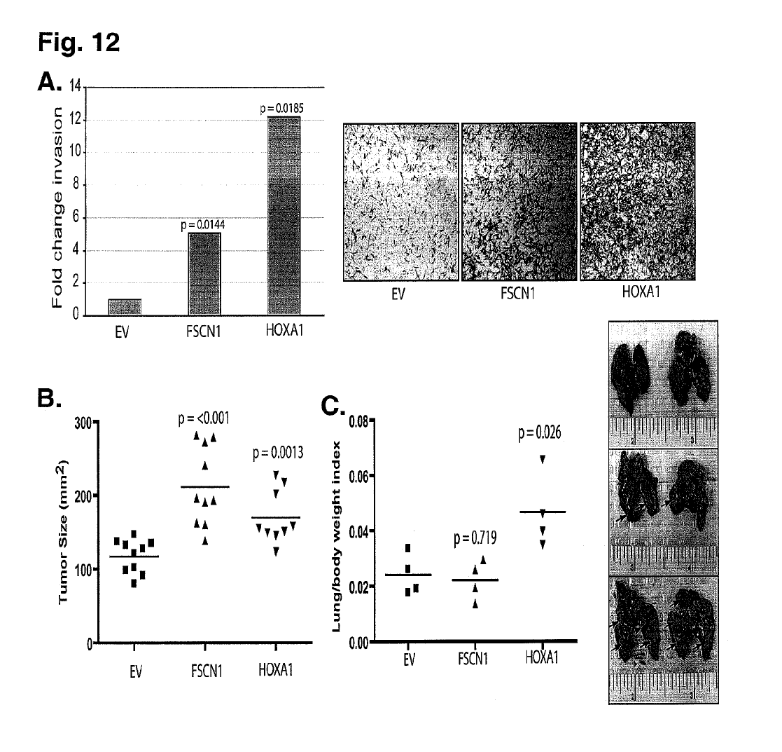

In addition to these studies in human cell lines, we also tested HOXA1 and Fascin 1 (FSCN1) using mouse cell lines. Consistent with invasion results using human cell systems (FIG. 11A-C), expression of both candidates markedly increased matrix invasion capability (FIG. 12A) of Ink4a/Ar^{f-/-} mouse-derive^{d} melanocytes transduced with HRAS* (know as M3HRAS cells, Kim, M., Gans, J. D., Nogueira, C., Wang, A., Paik, J. H., Feng, B., Brennan, C., Hahn, W. C., Cordon-Cardo, C., Wagner, S. N., et al. (2006). Comparative oncogenomics identifies NEDD9 as a melanoma metastasis gene. Cell 125, 1269-1281.). In addition, over-expression of both HOXA1 and Fascin 1 significantly enhanced the ability of M3HRAS cells to grow when xeno-transplanted onto the flanks of nude mice (FIG. 12B) and to form macroscopic lung nodules following intravenous tail vein injection, a surrogate assay for metastasis (FIG. 12C).

### Example 9

### HOXA1 is an Oncogene that can Promote Invasion Via Modulation of TGFβ Signaling

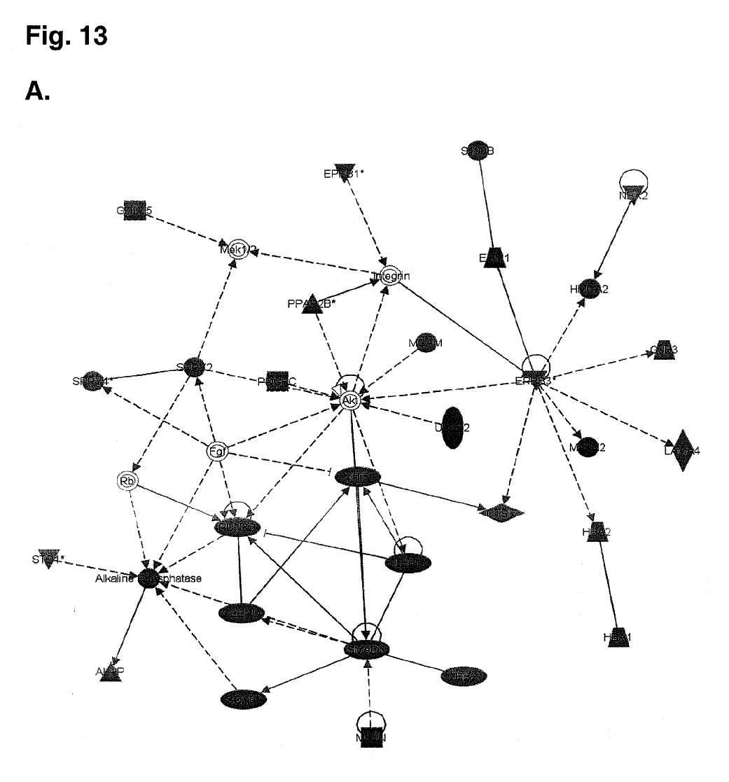

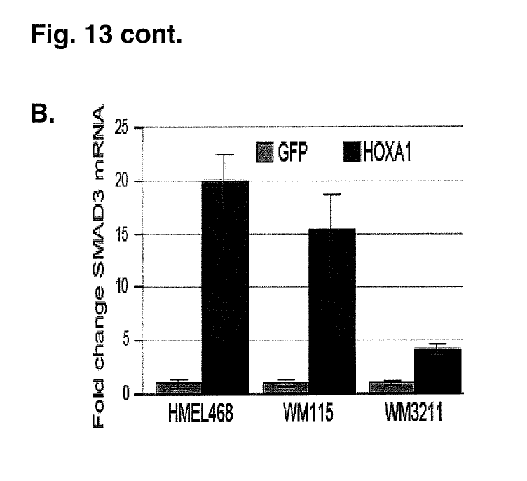

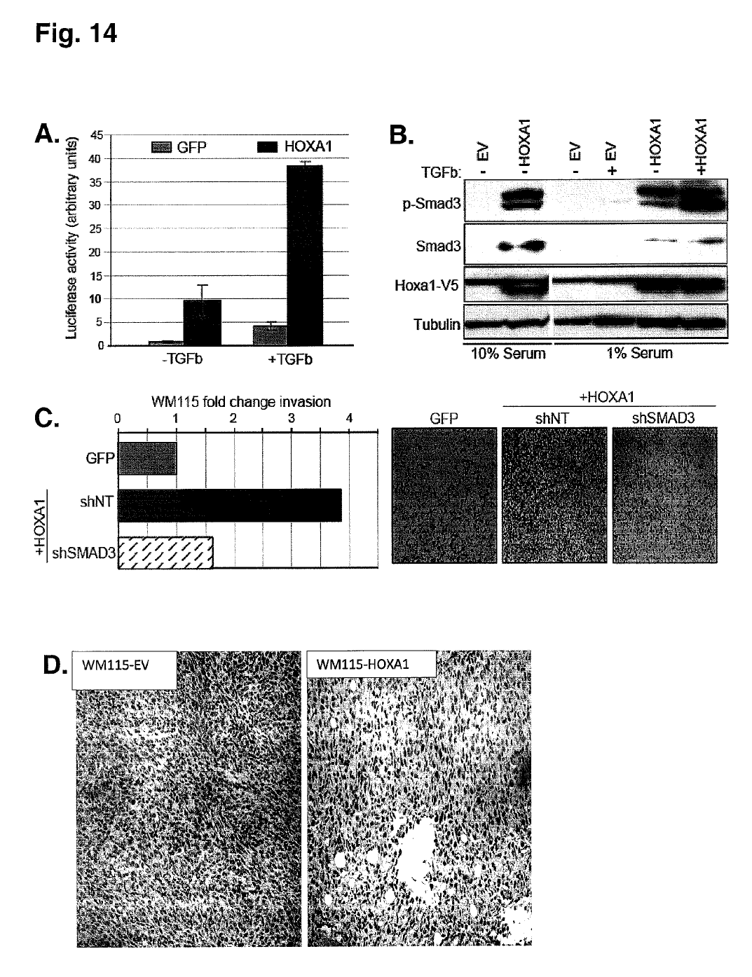

Next, to explore the molecular basis of HOXA1's invasive activity, we determined the HOXA1-transeriptome based on expression profiling of control and HOXA1-transduced HMEL468, WM115 and WM3211 cells (FIG. 11B). Knowledge-based pathway analysis of the differentially expressed gene list revealed a TGFβ signaling gene network centering on SMAD3 as a major node (FIG. 13A and Table 6.) Given its known role in metastasis²¹, we thus assessed whether TGFβ signaling was modulated by HOXA1. Using a TGFβ-responsive reporter construct (p3TP-Lux), we found that ectopic expression of HOXA1 not only enhanced basal reporter activity (11.0-fold, p=0.003), but also resulted in a 9.3 fold increase in response to TGFβ ligand compared to control (p=0.0001; FIG. 14A). Correspondingly, the activated p-SMAD3 and total SMAD3 were elevated under both 10% and 1% serum culturing conditions upon TGFβ stimulation (FIG. 14B), which was corroborated by RNA expression analysis (FIG. 13B). Moreover, HOXA1-mediated invasion was abrogated by knockdown of SMAD3 (FIG. 14C), thus functionally linking HOXA1's pro-invasion activity to TGFβ-SMAD signaling, a central pathway governing cancer metastasis²¹.

To examine whether HOXA1 over-expression influences SMAD3 phosphorylation status in tumors, we utilized xenograft tumors specimens derived from WM115 melanoma cells expressing empty vector or HOXA1 (FIG. 11E) for immunohistochemistry analysis using a phospho-specific antibody against SMAD3. Consistent with our observation that HOXA1 over-expression leads to increased phosphorylation of SMAD3 (FIG. 14B), we found increased SMAD3 phosphorylation in HOXA1 over-expressing tumors (FIG. 14D).

### Example

### CXCR4

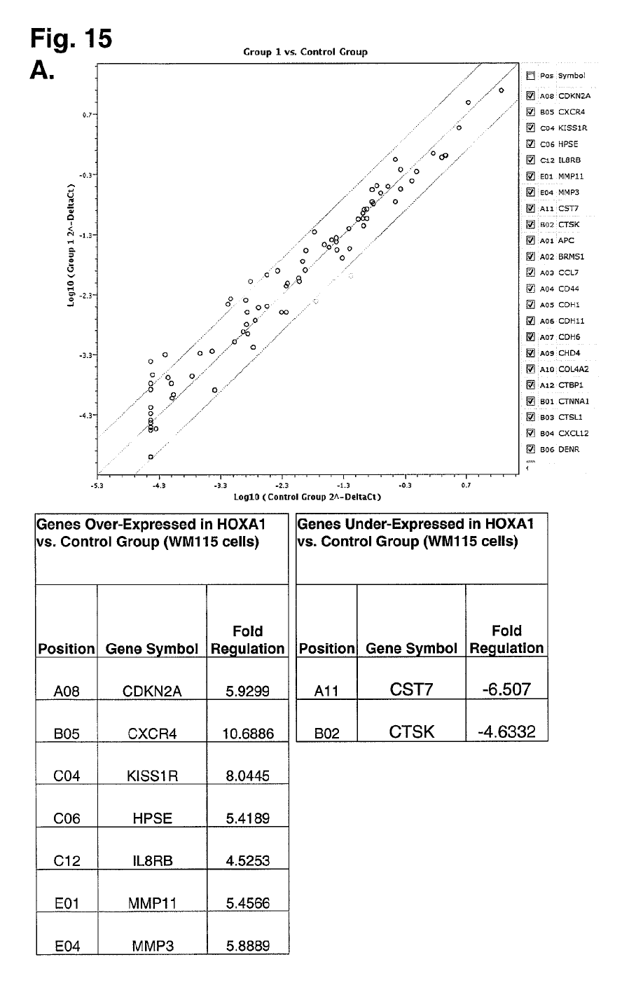

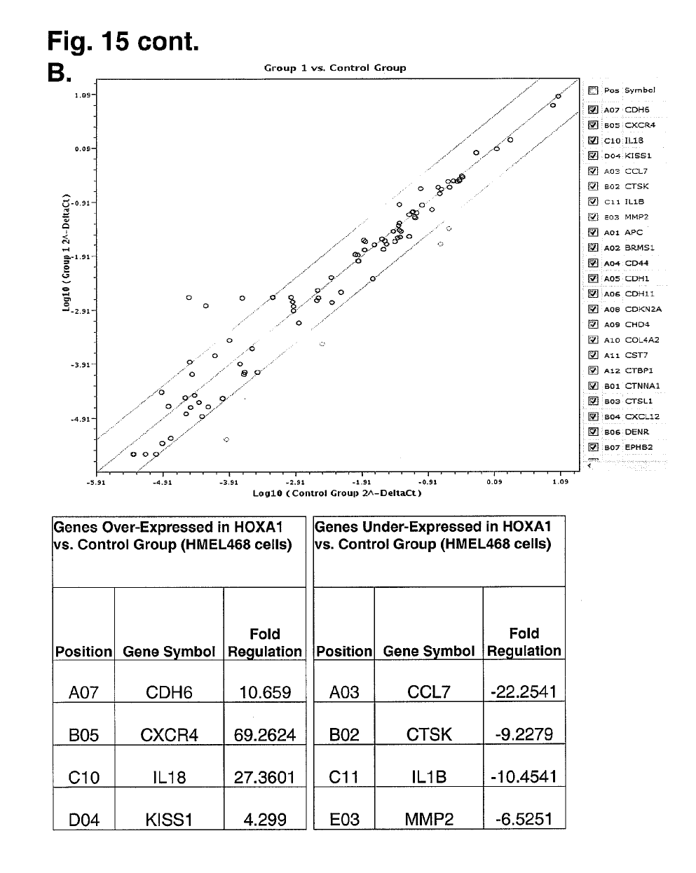

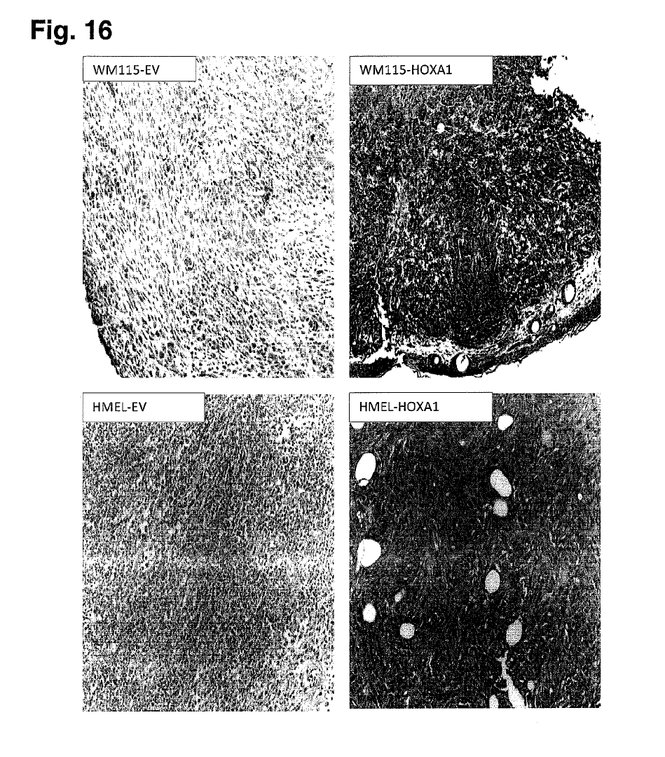

To gain insights into the biological functions of HOXA1, we prepared cDNA from empty vector and HOXA1 over-expressing WM115 melanoma cells and HMEL468 melanocytes for use on RT² Profiler PCR Arrays (Supperarray) to analyze expression of a panel of genes associated with metastasis. The top over-expressed gene shared between the two cell lines was the cemokine receptor CXCR4 (FIG. 15), a receptor specific for chemokine stromal-derived-factor-1 (SDF-1). CXCR4 expression by tumor cells has been correlated with poor prognosis in many types of cancer and plays a critical role in cell metastasis through establishment of a chemotactic gradient to organs expressing SDF-1 (Fulton AM. Curr Oncol Rep. 2009 March; 11(2):125-31). To further examine the relationship between HOXA1 and CXCR4, we assessed the CXCR4 expression in empty vector- and HOXA1-over-expressing xenograft tumors using immunohistochemistry. Consistent with the RT² Profiler analysis, we found that CXCR4 expression was markedly increased in both WM115-HOXA1 and HMEL468-HOXA1 xenograft tumors (FIG. 16). These data are consistent with a model whereby HOXA1 leads to increased expression of CXCR4, which in turn influences metastatic signaling programs initiated by over-expression of HOXA1

In summary, an integrative functional genomics approach has enabled the identification of metastasis determinants that are both active drivers of invasion and bona fide oncogenes. These metastasis determinants, discovered in the context of melanoma, proved prognostic in early stage breast adenocarcinomas and showed progression-correlated expression in diverse non-melanoma tumor types. These findings provide experimental evidence that metastasis determinants are present in some early-stage primary tumors and can program these tumors to behave aggressively, therefore confer poor clinical outcome. As the majority of these determinants have not been linked to cancer or metastasis, they may provide a basis for functionally-based prognostic biomarkers and new therapeutic inroads.

TABLE 3

| | | Invasion | | | | | | | |
|---|---|---|---|---|---|---|---|---|---|
| Gene | | Screen | Invasion Assay | | Oncomine Correlation | | | | |
| Symbols | Gene ID | HMEL468 | WM115 | WM3211 | Melanoma | Brain | Breast | Prostate | Lung |
| ACP5 | 54 | 6.5X | 2.1X | no increase | | | | + | |
| ANLN | 54443 | 2.6X | — | 2.6X | | | + | | |
| ASF1B | 55723 | 4.7X | 2.0X | 2.3X | + | + | + | + | |
| BRRN1 | 23397 | 3.5X | 2.1X | 4.0X | + | | + | + | |
| BUB1 | 699 | 3.1X | — | — | + | | + | | |
| CDC2 | 983 | 1.6X | — | — | + | + | + | + | + |
| CENPM | 79019 | 6.9X | — | — | | | + | + | |
| DEPDC1 | 55635 | 2.3X | — | — | | + | + | | |
| ELTD1 | 64123 | 2.1X | 5.0X | no increase | | + | | | |
| EXT1 | 2131 | 1.5X | — | — | | + | + | | |
| FSCN1 | 6624 | 2.4X | 2.2X | 1.8X | | | | + | + |
| HCAP-G | 64151 | 1.5X | — | — | + | + | + | | |
| HMGB1 | 3146 | 3.4X | — | — | + | | | + | + |
| HMGB2 | 3148 | 1.5X | — | — | | + | + | + | |
| HOXA1 | 3198 | 7.8X | 6.1X | 5.1X | | + | | | |
| HSF1 | 3297 | 2.8X | 4.4X | — | + | | + | + | |
| ITGB3BP | 23421 | 4.2X | — | — | | + | | | |
| KIF20A | 10112 | 1.5X | — | — | + | + | + | | |
| KIF2C | 11004 | 1.6X | — | — | + | + | + | | |
| KNTC2 | 10403 | 2.4X | 2.2X | 3.5X | | + | + | | |
| MCM7 | 4176 | 9.4X | — | — | + | + | + | | |
| MTHFD2 | 10797 | 2.4X | 2.5X | — | | | + | + | + |
| NASP | 4678 | 3.7X | — | — | | + | + | | |
| PLVAP | 83483 | 1.5X | — | — | | | + | | + |
| PTP4A3 | 11156 | 1.9X | — | — | + | + | + | + | |
| RNF2 | 6045 | 2.9X | 3.4X | 5.7X | | | | | + |
| SPAG5 | 10615 | 3.7X | 2.5X | 3.1X | + | | + | | |
| TGM2 | 7052 | 1.7X | — | — | + | | | | + |
| UBE2C | 11065 | 3.9X | — | — | + | + | + | + | + |
| UCHL5 | 51377 | 4.1X | no | 1.9X | | | + | | |
| | | | increase | | | | | | |
| VSIG4 | 11326 | 4.8X | 2.1X | 1.5X | | + | | | |

TABLE 4
Summary of integrated dataset comprising 360
potential metastasis determinants.

| | |
|---|---|
| 65 under-expressed/ | 295 over-expressed/ |
| deleted candidates | amplified candidates |

| Gene ID | Gene Symbol | Gene ID | Gene Symbol |
|---|---|---|---|
| 79026 | AHNAK | 79575 | ABHD8 |
| 360 | AQP3 | 1636 | ACE |
| 622 | BDH1 | 54 | ACP5 |
| 219738 | C10ORF35 | 8038 | ADAM12 |
| 726 | CAPN5 | 101 | ADAM8 |
| 999 | CDH1 | 23600 | AMACR |
| 51673 | CGI-38 | 54443 | ANLN |
| 1159 | CKMT1B | 80833 | APOL3 |
| 85445 | CNTNAP4 | 410 | ARSA |
| 1303 | COL12A1 | 22901 | ARSG |
| 9244 | CRLF1 | 55723 | ASF1B |
| 1410 | CRYAB | 259266 | ASPM |
| 1428 | CRYM | 477 | ATP1A2 |
| 113878 | DTX2 | 6790 | AURKA |
| 10278 | EFS | 9212 | AURKB |
| 79993 | ELOVL7 | 26053 | AUTS2 |
| 2041 | EPHA1 | 627 | BDNF |
| 2045 | EPHA7 | 638 | BIK |
| 2051 | EPHB6 | 332 | BIRC5 |
| 10205 | EVA1 | 672 | BRCA1 |
| 2125 | EVPL | 23397 | BRRN1 |
| 2159 | F10 | 699 | BUB1 |
| 375061 | FAM89A | 701 | BUB1B |
| 8857 | FCGBP | 80135 | BXDC5 |
| 2261 | FGFR3 | 29902 | C12ORF24 |
| 56776 | FMN2 | 55839 | C16ORF60 |
| 2770 | GNAI1 | 56942 | C16ORF61 |
| 7107 | GPR137B | 116496 | C1ORF24 |
| 64388 | GREM2 | 719 | C3AR1 |
| 3098 | HK1 | 57002 | C7ORF36 |
| 688 | KLF5 | 84933 | C8ORF76 |
| 5655 | KLK10 | 152007 | C9ORF19 |
| 11202 | KLK8 | 781 | CACNA2D1 |
| 10748 | KLRA1 | 857 | CAV1 |
| 10219 | KLRG1 | 6357 | CCL13 |
| 4135 | MAP6 | 6347 | CCL2 |
| 5603 | MAPK13 | 6354 | CCL7 |
| 4312 | MMP1 | 890 | CCNA2 |
| 4486 | MST1R | 947 | CD34 |
| 4692 | NDN | 948 | CD36 |
| 5092 | PCBD1 | 983 | CDC2 |
| 10158 | PDZK1IP1 | 991 | CDC20 |
| 5317 | PKP1 | 995 | CDC25C |
| 26499 | PLEK2 | 990 | CDC6 |
| 58473 | PLEKHB1 | 8317 | CDC7 |
| 5366 | PMAIP1 | 83540 | CDCA1 |
| 79983 | POF1B | 83461 | CDCA3 |
| 5453 | POU3F1 | 55536 | CDCA7L |
| 5579 | PRKCB1 | 81620 | CDT1 |
| 5745 | PTHR1 | 1058 | CENPA |
| 5792 | PTPRF | 1062 | CENPE |
| 57111 | RAB25 | 1063 | CENPF |
| 6095 | RORA | 79019 | CENPM |
| 6337 | SCNN1A | 55165 | CEP55 |
| 6382 | SDC1 | 23177 | CEP68 |
| 5268 | SERPINB5 | 1070 | CETN3 |
| 11254 | SLC6A14 | 1111 | CHEK1 |
| 6578 | SLCO2A1 | 26586 | CKAP2 |
| 6586 | SLIT3 | 1163 | CKS1B |
| 10653 | SPINT2 | 1164 | CKS2 |
| 6768 | ST14 | 1180 | CLCN1 |
| 7070 | THY1 | 7122 | CLDN5 |
| 23650 | TRIM29 | 23601 | CLEC5A |
| 23555 | TSPAN15 | 9918 | CNAP1 |
| 11197 | WIF1 | 10664 | CTCF |
| | | 1565 | CYP2D6 |
| | | 1601 | DAB2 |
| | | 10926 | DBF4 |
| | | 23564 | DDAH2 |
| | | 55635 | DEPDC1 |
| | | 1719 | DHFR |
| | | 55355 | DKFZP762E1312 |
| | | 27122 | DKK3 |
| | | 9787 | DLG7 |
| | | 1769 | DNAH8 |
| | | 30836 | DNTTIP2 |
| | | 51514 | DTL |
| | | 1854 | DUT |
| | | 1894 | ECT2 |
| | | 51162 | EGFL7 |
| | | 64123 | ELTD1 |
| | | 56943 | ENY2 |
| | | 54749 | EPDR1 |
| | | 51327 | ERAF |
| | | 2115 | ETV1 |
| | | 2131 | EXT1 |
| | | 2162 | F13A1 |
| | | 51647 | FAM96B |
| | | 2230 | FDX1 |
| | | 2235 | FECH |
| | | 63979 | FIGNL1 |
| | | 51303 | FKBP11 |
| | | 2289 | FKBP5 |
| | | 55110 | FLJ10292 |
| | | 79805 | FLJ12505 |
| | | 84935 | FLJ14834 |
| | | 54908 | FLJ20364 |
| | | 54962 | FLJ20516 |
| | | 2350 | FOLR2 |
| | | 2305 | FOXM1 |
| | | 6624 | FSCN1 |
| | | 2530 | FUT8 |
| | | 51809 | GALNT7 |
| | | 64096 | GFRA4 |
| | | 2740 | GLP1R |
| | | 51053 | GMNN |
| | | 2775 | GNAO1 |
| | | 2792 | GNGT1 |
| | | 4076 | GPIAP1 |
| | | 2894 | GRID1 |
| | | 2936 | GSR |
| | | 2966 | GTF2H2 |
| | | 51512 | GTSE1 |
| | | 3045 | HBD |
| | | 64151 | HCAP-G |
| | | 50810 | HDGFRP3 |
| | | 3082 | HGF |
| | | 3012 | HIST1H2AB |
| | | 3142 | HLX1 |
| | | 3146 | HMGB1 |
| | | 3148 | HMGB2 |
| | | 3161 | HMMR |
| | | 10236 | HNRPR |
| | | 3198 | HOXA1 |
| | | 10247 | HRSP12 |
| | | 3297 | HSF1 |
| | | 3313 | HSPA9B |
| | | 51501 | HSPC138 |
| | | 10808 | HSPH1 |
| | | 25998 | IBTK |
| | | 3384 | ICAM2 |
| | | 80173 | IFT74 |
| | | 3570 | IL6R |
| | | 3684 | ITGAM |
| | | 23421 | ITGB3BP |
| | | 6453 | ITSN1 |
| | | 10008 | KCNE3 |
| | | 3776 | KCNK2 |
| | | 9768 | KIAA0101 |
| | | 9694 | KIAA0103 |
| | | 56243 | KIAA1217 |
| | | 84629 | KIAA1856 |
| | | 3832 | KIF11 |
| | | 81930 | KIF18A |
| | | 10112 | KIF20A |
| | | 11004 | KIF2C |
| | | 3833 | KIFC1 |
| | | 55220 | KLHDC8A |
| | | 10403 | KNTC2 |
| | | 3912 | LAMB1 |
| | | 3915 | LAMC1 |
| | | 55915 | LANCL2 |
| | | 11025 | LILRB3 |
| | | 4005 | LMO2 |
| | | 150084 | LOC150084 |
| | | 345711 | LOC345711 |
| | | 91614 | LOC91614 |
| | | 26018 | LRIG1 |
| | | 54892 | LUZP5 |
| | | 4085 | MAD2L1 |
| | | 6300 | MAPK12 |
| | | 4147 | MATN2 |
| | | 4172 | MCM3 |
| | | 4174 | MCM5 |
| | | 4175 | MCM6 |
| | | 4176 | MCM7 |
| | | 9833 | MELK |
| | | 4232 | MEST |
| | | 4233 | MET |
| | | 85014 | MGC14141 |
| | | 79971 | MIER1 |
| | | 4288 | MKI67 |
| | | 8028 | MLLT10 |
| | | 4317 | MMP8 |
| | | 4318 | MMP9 |
| | | 4353 | MPO |
| | | 51678 | MPP6 |
| | | 219928 | MRGPRF |
| | | 64968 | MRPS6 |
| | | 10335 | MRVI1 |
| | | 10232 | MSLN |
| | | 10797 | MTHFD2 |
| | | 4600 | MX2 |
| | | 4678 | NASP |
| | | 4751 | NEK2 |
| | | 23530 | NNT |
| | | 4846 | NOS3 |
| | | 4855 | NOTCH4 |
| | | 84955 | NUDCD1 |
| | | 11163 | NUDT4 |
| | | 53371 | NUP54 |
| | | 4928 | NUP98 |
| | | 51203 | NUSAP1 |
| | | 4999 | ORC2L |
| | | 116039 | OSR2 |
| | | 5019 | OXCT1 |
| | | 56288 | PARD3 |
| | | 55872 | PBK |
| | | 11333 | PDAP1 |
| | | 5138 | PDE2A |
| | | 5156 | PDGFRA |
| | | 5175 | PECAM1 |
| | | 5218 | PFTK1 |
| | | 25776 | PGEA1 |
| | | 26227 | PHGDH |
| | | 83483 | PLVAP |
| | | 57125 | PLXDC1 |
| | | 5425 | POLD2 |
| | | 5427 | POLE2 |
| | | 5446 | PON3 |
| | | 5557 | PRIM1 |
| | | 5558 | PRIM2A |
| | | 5578 | PRKCA |
| | | 23627 | PRND |
| | | 9265 | PSCD3 |
| | | 5743 | PTGS2 |
| | | 11156 | PTP4A3 |
| | | 5885 | RAD21 |
| | | 5888 | RAD51 |
| | | 5889 | RAD51C |
| | | 3516 | RBPSUH |
| | | 5965 | RECQL |
| | | 5984 | RFC4 |
| | | 5985 | RFC5 |
| | | 23179 | RGL1 |
| | | 64407 | RGS18 |
| | | 5997 | RGS2 |
| | | 8490 | RGS5 |
| | | 6045 | RNF2 |
| | | 9584 | RNPC2 |
| | | 6091 | ROBO1 |
| | | 6118 | RPA2 |
| | | 6119 | RPA3 |
| | | 6222 | RPS18 |
| | | 6236 | RRAD |
| | | 22800 | RRAS2 |
| | | 6240 | RRM1 |
| | | 6241 | RRM2 |
| | | 340419 | RSPO2 |
| | | 10371 | SEMA3A |
| | | 143686 | SESN3 |
| | | 85358 | SHANK3 |
| | | 79801 | SHCBP1 |
| | | 8036 | SHOC2 |
| | | 23517 | SKIV2L2 |
| | | 7884 | SLBP |
| | | 6509 | SLC1A4 |
| | | 115286 | SLC25A26 |
| | | 6526 | SLC5A3 |
| | | 8467 | SMARCA5 |
| | | 8243 | SMC1L1 |
| | | 10592 | SMC2L1 |
| | | 10051 | SMC4L1 |
| | | 6629 | SNRPB2 |
| | | 64321 | SOX17 |
| | | 6662 | SOX9 |
| | | 10615 | SPAG5 |
| | | 57405 | SPBC25 |
| | | 60559 | SPCS3 |
| | | 6741 | SSB |
| | | 6742 | SSBP1 |
| | | 26872 | STEAP1 |
| | | 6788 | STK3 |
| | | 10460 | TACC3 |
| | | 23435 | TARDBP |
| | | 25771 | TBC1D22A |
| | | 6899 | TBX1 |
| | | 7052 | TGM2 |
| | | 90390 | THRAP6 |
| | | 8914 | TIMELESS |
| | | 7077 | TIMP2 |
| | | 7083 | TK1 |
| | | 55273 | TMEM100 |
| | | 55161 | TMEM33 |
| | | 55706 | TMEM48 |
| | | 54543 | TOMM7 |
| | | 7153 | TOP2A |
| | | 22974 | TPX2 |
| | | 54209 | TREM2 |
| | | 4591 | TRIM37 |
| | | 9319 | TRIP13 |
| | | 95681 | TSGA14 |
| | | 11065 | UBE2C |
| | | 51377 | UCHL5 |
| | | 7371 | UCK2 |
| | | 83878 | USHBP1 |
| | | 11326 | VSIG4 |
| | | 10894 | XLKD1 |
| | | 51776 | ZAK |
| | | 221527 | ZBTB12 |
| | | 346171 | ZFP57 |
| | | 23414 | ZFPM2 |
| | | 79830 | ZMYM1 |
| | | 7705 | ZNF146 |
| | | 84858 | ZNF503 |

TABLE 5
Candidate cDNAs screened and primary hits identified in
the low complexity genetic screen for pro-invasion genes.

| | | 46 primary screen 46 hits |
|---|---|---|
| 199 candidates screened | | (2xSD in 2 screens) |
| Gene ID | Gene Symbol | Gene Symbol |
| 54 | ACP5 | ACP5 |
| 54443 | ANLN | ANLN |
| 410 | ARSA | ARSA |
| 55723 | ASF1B | ASF1B |
| 9212 | AURKB | AURKB |
| 23397 | BRRN1 | BRRN1 |
| 80135 | BXDC5 | BXDC5 |
| 947 | CD34 | CD34 |
| 55536 | CDCA7L | CDCA7L |
| 79019 | CENPM | CENPM |
| 55635 | DEPDC1 | DEPDC1 |
| 51514 | DTL | DTL |
| 64123 | ELTD1 | ELTD1 |
| 2131 | EXT1 | EXT1 |
| 63979 | FIGNL1 | FIGNL1 |
| 55220 | KLHDC8A | FLJ10748 |
| 6624 | FSCN1 | FSCN1 |
| 2775 | GNAO1 | GNAO1 |
| 2792 | GNGT1 | GNGT1 |
| 2894 | GRID1 | GRID1 |
| 50810 | HDGFRP3 | HDGFRP3 |
| 3146 | HMGB1 | HMGB1 |
| 3148 | HMGB2 | HMGB2 |
| 3198 | HOXA1 | HOXA1 |
| 3297 | HSF1 | HSF1 |
| 10808 | HSPH1 | HSPH1 |
| 23421 | ITGB3BP | ITGB3BP |
| 10403 | KNTC2 | KNTC2 |
| 4174 | MCM5 | MCM5 |
| 4176 | MCM7 | MCM7 |
| 10797 | MTHFD2 | MTHFD2 |
| 4855 | NOTCH4 | NOTCH4 |
| 53371 | NUP54 | NUP54 |
| 4999 | ORC2L | ORC2L |
| 83483 | PLVAP | PLVAP |
| 9265 | PSCD3 | PSCD3 |
| 6045 | RNF2 | RNF2 |
| 10615 | SPAG5 | SPAG5 |
| 26872 | STEAP1 | STEAP1 |
| 7052 | TGM2 | TGM2 |
| 54543 | TOMM7 | TOMM7 |
| 22974 | TPX2 | TPX2 |
| 11065 | UBE2C | UBE2C |
| 51377 | UCHL5 | UCHL5 |
| 11326 | VSIG4 | VSIG4 |
| 23600 | AMACR | |
| 10926 | DBF4 | |
| 259266 | ASPM | |
| 477 | ATP1A2 | |
| 627 | BDNF | |
| 638 | BIK | |
| 332 | BIRC5 | |
| 55839 | C16ORF60 | |
| 672 | BRCA1 | |
| 699 | BUB1 | |
| 701 | BUB1B | |
| 55165 | CEP55 | |
| 79971 | MIER1 | |
| 116496 | C1ORF24 | |
| 719 | C3AR1 | |
| 57002 | C7ORF36 | |
| 84933 | C8ORF76 | |
| 152007 | C9ORF19 | |
| 857 | CAV1 | |
| 6357 | CCL13 | |
| 6347 | CCL2 | |
| 948 | CD36 | |
| 983 | CDC2 | |
| 991 | CDC20 | |
| 995 | CDC25C | |
| 83540 | CDCA1 | |
| 83461 | CDCA3 | |
| 1058 | CENPA | |
| 1070 | CETN3 | |
| 26586 | CKAP2 | |
| 1163 | CKS1B | |
| 1164 | CKS2 | |
| 9918 | CNAP1 | |
| 10664 | CTCF | |
| 1601 | DAB2 | |
| 56942 | C16ORF61 | |
| 23564 | DDAH2 | |
| 1719 | DHFR | |
| 55355 | DKFZP762E1312 | |
| 27122 | DKK3 | |
| 9787 | DLG7 | |
| 30836 | DNTTIP2 | |
| 1854 | DUT | |
| 51162 | EGFL7 | |
| 56943 | ENY2 | |
| 54749 | EPDR1 | |
| 2162 | F13A1 | |
| 51303 | FKBP11 | |
| 55110 | FLJ10292 | |
| 55273 | TMEM100 | |
| 79805 | FLJ12505 | |
| 84935 | FLJ14834 | |
| 54962 | FLJ20516 | |
| 2305 | FOXM1 | |
| 51809 | GALNT7 | |
| 51053 | GMNN | |
| 2936 | GSR | |
| 2966 | GTF2H2 | |
| 51512 | GTSE1 | |
| 3045 | HBD | |
| 64151 | HCAP-G | |
| 3082 | HGF | |
| 3142 | HLX1 | |
| 10236 | HNRPR | |
| 10247 | HRSP12 | |
| 3313 | HSPA9B | |
| 51501 | HSPC138 | |
| 29902 | C12ORF24 | |
| 3384 | ICAM2 | |
| 10008 | KCNE3 | |
| 9768 | KIAA0101 | |
| 9694 | KIAA0103 | |
| 23177 | CEP68 | |
| 22901 | ARSG | |
| 56243 | KIAA1217 | |
| 10112 | KIF20A | |
| 11004 | KIF2C | |
| 3915 | LAMC1 | |
| 55915 | LANCL2 | |
| 4005 | LMO2 | |
| 91614 | LOC91614 | |
| 4076 | GPIAP1 | |
| 4085 | MAD2L1 | |
| 6300 | MAPK12 | |
| 4172 | MCM3 | |
| 4175 | MCM6 | |
| 4232 | MEST | |
| 85014 | MGC14141 | |
| 4318 | MMP9 | |
| 219928 | MRGPRF | |
| 64968 | MRPS6 | |
| 10232 | MSLN | |
| 4600 | MX2 | |
| 4678 | NASP | |
| 4751 | NEK2 | |
| 23530 | NNT | |
| 4846 | NOS3 | |
| 11163 | NUDT4 | |
| 51203 | NUSAP1 | |
| 116039 | OSR2 | |
| 5019 | OXCT1 | |
| 56288 | PARD3 | |
| 55872 | PBK | |
| 11333 | PDAP1 | |
| 5156 | PDGFRA | |
| 25776 | PGEA1 | |
| 57125 | PLXDC1 | |
| 5425 | POLD2 | |
| 5446 | PON3 | |
| 5557 | PRIM1 | |
| 5558 | PRIM2A | |
| 23627 | PRND | |
| 5743 | PTGS2 | |
| 11156 | PTP4A3 | |
| 5885 | RAD21 | |
| 5889 | RAD51C | |
| 3516 | RBPSUH | |
| 5965 | RECQL | |
| 5984 | RFC4 | |
| 5985 | RFC5 | |
| 64407 | RGS18 | |
| 5997 | RGS2 | |
| 8490 | RGS5 | |
| 6118 | RPA2 | |
| 6119 | RPA3 | |
| 6236 | RRAD | |
| 22800 | RRAS2 | |
| 6240 | RRM1 | |
| 6241 | RRM2 | |
| 340419 | RSPO2 | |
| 79801 | SHCBP1 | |
| 8036 | SHOC2 | |
| 7884 | SLBP | |
| 115286 | SLC25A26 | |
| 8467 | SMARCA5 | |
| 6629 | SNRPB2 | |
| 57405 | SPBC25 | |
| 60559 | SPCS3 | |
| 6742 | SSBP1 | |
| 6788 | STK3 | |
| 23435 | TARDBP | |
| 25771 | TBC1D22A | |
| 90390 | THRAP6 | |
| 8914 | TIMELESS | |
| 7077 | TIMP2 | |
| 7083 | TK1 | |
| 4591 | TRIM37 | |
| 9319 | TRIP13 | |
| 7371 | UCK2 | |
| 83878 | USHBP1 | |
| 10894 | XLKD1 | |
| 51776 | ZAK | |
| 79830 | ZMYM1 | |
| 84858 | ZNF503 | |

TABLE 6
Complete description of the genes in the Smad3-related biological network in FIG. 13A

| Gene ID | Name | Description | Family |
|---|---|---|---|
| | Akt | | group |
| | Alkaline | | group |
| | Phosphatase | | |
| 250 | ALPP | alkaline phosphatase, placental (Regan | phosphatase |
| | | isozyme) | |
| 1052 | CEBPD | CCAAT/enhancer binding protein (C/EBP), delta | transcription regulator |
| 1513 | CTSK | cathepsin K | peptidase |
| 1893 | ECM1 | extracellular matrix protein 1 | transporter |
| 2047 | EPHB1 | EPH receptor B1 | kinase |
| 2065 | ERBB3 | v-erb-b2 erythroblastic leukemia viral oncogene 3 | kinase |
| | Fgf | | group |
| 9518 | GDF15 | growth differentiation factor 15 | growth factor |
| 2707 | GJB3 | gap junction protein, beta 3, 31 kDa | transporter |
| 3039 | HBA1 | hemoglobin, alpha 1 | transporter |
| 3040 | HBA2 | hemoglobin, alpha 2 | transporter |
| 8091 | HMGA2 | high mobility group AT-hook 2 | other |
| | Integrin | | complex |
| 3910 | LAMA4 | laminin, alpha 4 | enzyme |
| 51176 | LEF1 | lymphoid enhancer-binding factor 1 | transcription regulator |
| 4147 | MATN2 | matrilin 2 | other |
| 4162 | MCAM | melanoma cell adhesion molecule | other |
| | Mek1/2 | | group |
| 4286 | MITF | microphthalmia-associated transcription factor | transcription regulator |
| 2660 | MSTN | myostatin | growth factor |
| 4751 | NEK2 | NIMA (never in mitosis gene a)-related kinase 2 | kinase |
| 56034 | PDGFC | platelet derived growth factor C | growth factor |
| 8613 | PPAP2B | phosphatidic acid phosphatase type 2B | phosphatase |
| | Rb | | group |
| 860 | RUNX2 | runt-related transcription factor 2 | transcription regulator |
| 6285 | S100B | S100 calcium binding protein B | other |
| 4088 | SMAD3 | SMAD family member 3 | transcription regulator |
| 6662 | SOX9 | SRY (sex determining region Y)-box 9 | transcription regulator |
| 10253 | SPRY2 | sprouty homolog 2 (Drosophila) | other |
| 81848 | SPRY4 | sprouty homolog 4 (Drosophila) | other |
| 6781 | STC1 | stanniocalcin 1 | kinase |
| 80328 | ULBP2 | UL16 binding protein 2 | transmembrane |
| | | | receptor |
| 9839 | ZEB2 | zinc finger E-box binding homeobox 2 | transcription regulator |

TABLE 7
K-mean class assignment of published 295 breast cancer cases^{5;6}.

| Pateint | Overall Survival | | Metastasis-free Survival | | k mean |
|---|---|---|---|---|---|
| ID | Time | status | time | status | group |
| 4 | 12.9965777 | alive | 12.99658 | no metastasis | 2 |
| 6 | 11.156742 | alive | 11.15674 | no metastasis | 2 |
| 7 | 10.1382615 | alive | 10.13826 | no metastasis | 2 |
| 8 | 8.80219028 | alive | 8.80219 | no metastasis | 1 |
| 9 | 10.294319 | alive | 10.29432 | no metastasis | 2 |
| 11 | 5.80424367 | alive | 5.804244 | no metastasis | 1 |
| 12 | 7.85763176 | alive | 7.857632 | no metastasis | 1 |
| 13 | 8.1670089 | alive | 8.167009 | no metastasis | 1 |
| 14 | 8.23271732 | alive | 8.232717 | no metastasis | 2 |
| 17 | 7.86584531 | alive | 7.865845 | no metastasis | 2 |
| 26 | 6.9705681 | alive | 6.970568 | no metastasis | 2 |
| 27 | 5.18548939 | alive | 5.185489 | no metastasis | 2 |
| 28 | 6.24503765 | alive | 6.245038 | no metastasis | 2 |
| 29 | 11.3894593 | alive | 11.38946 | no metastasis | 2 |
| 36 | 10.1081451 | alive | 10.10815 | no metastasis | 2 |
| 38 | 7.35386721 | alive | 7.353867 | no metastasis | 2 |
| 39 | 11.0171116 | alive | 11.01711 | no metastasis | 2 |
| 45 | 4.7315 | alive | 1.089665 | metastasis | 1 |
| 48 | 2.1726 | dead | 1.026694 | metastasis | 1 |
| 51 | 9.526 | dead | 4.906229 | metastasis | 2 |
| 56 | 8.4658 | dead | 4.695414 | metastasis | 2 |
| 57 | 5.1508 | dead | 2.297057 | metastasis | 1 |
| 58 | 5.3562 | dead | 1.122519 | metastasis | 1 |
| 59 | 4.9946 | alive | 4.629706 | metastasis | 1 |
| 60 | 7.9288 | dead | 4.892539 | metastasis | 2 |
| 61 | 4.1178 | alive | 2.680356 | metastasis | 2 |
| 62 | 2.7096 | dead | 0.807666 | metastasis | 1 |
| 71 | 2.6083 | dead | 1.982204 | metastasis | 1 |
| 72 | 5.5041 | dead | 3.028063 | metastasis | 1 |
| 73 | 2.6192 | dead | 2.149213 | metastasis | 2 |
| 75 | 2.2905 | dead | 2.209446 | metastasis | 1 |
| 76 | 3.737 | dead | 2.12731 | metastasis | 2 |
| 103 | 5.77960301 | dead | 4.952772 | metastasis | 1 |
| 107 | 3.45516769 | dead | 2.543463 | metastasis | 1 |
| 109 | 3.225188 | alive | 3.195072 | metastasis | 1 |
| 110 | 2.310746 | alive | 2.168378 | metastasis | 1 |
| 111 | 3.25256674 | dead | 1.270363 | metastasis | 1 |
| 113 | 3.24161533 | dead | 0.996578 | metastasis | 2 |
| 117 | 5.30321698 | alive | 5.303217 | no metastasis | 2 |
| 118 | 5.23203285 | alive | 5.232033 | no metastasis | 2 |
| 120 | 10.0971937 | alive | 10.09719 | no metastasis | 2 |
| 122 | 14.8172485 | alive | 14.81725 | no metastasis | 2 |
| 123 | 14.2614648 | alive | 14.26146 | no metastasis | 2 |
| 124 | 6.64476386 | alive | 6.644764 | no metastasis | 2 |
| 125 | 7.74811773 | alive | 7.748118 | no metastasis | 2 |
| 126 | 6.4366872 | alive | 6.31896 | metastasis | 1 |
| 127 | 5.03764545 | alive | 4.66256 | metastasis | 1 |
| 128 | 8.73921971 | alive | 8.73922 | no metastasis | 1 |
| 129 | 7.56741958 | alive | 7.56742 | no metastasis | 2 |
| 130 | 7.29637235 | alive | 7.296372 | no metastasis | 1 |
| 131 | 4.66255989 | dead | 4.66256 | no metastasis | 1 |
| 132 | 6.71868583 | alive | 6.718686 | no metastasis | 1 |
| 133 | 8.64887064 | alive | 8.648871 | no metastasis | 2 |
| 134 | 7.09377139 | dead | 6.995209 | metastasis | 2 |
| 135 | 9.33059548 | alive | 9.330595 | no metastasis | 1 |
| 136 | 3.8220397 | dead | 3.438741 | metastasis | 1 |
| 137 | 15.3292266 | alive | 15.32923 | no metastasis | 2 |
| 138 | 3.84941821 | dead | 3.474333 | metastasis | 2 |
| 139 | 12.7665982 | alive | 12.7666 | no metastasis | 1 |
| 140 | 5.55509925 | alive | 5.555099 | no metastasis | 2 |
| 141 | 2.06433949 | dead | 1.40178 | metastasis | 1 |
| 142 | 15.1348392 | alive | 15.13484 | no metastasis | 2 |
| 144 | 14.1273101 | alive | 14.12731 | no metastasis | 1 |
| 145 | 5.48665298 | alive | 5.486653 | no metastasis | 2 |
| 146 | 9.40725531 | dead | 3.655031 | metastasis | 2 |
| 147 | 2.70773443 | dead | 1.609856 | metastasis | 1 |
| 148 | 18.3408624 | alive | 18.34086 | no metastasis | 2 |
| 149 | 17.2402464 | alive | 17.24025 | no metastasis | 1 |
| 150 | 1.48665298 | dead | 0.960986 | metastasis | 1 |
| 151 | 17.5742642 | alive | 14.01232 | metastasis | 2 |
| 153 | 3.03627652 | dead | 1.177276 | metastasis | 1 |
| 154 | 15.1047228 | alive | 15.10472 | no metastasis | 2 |
| 155 | 1.84804928 | dead | 0.930869 | metastasis | 2 |
| 156 | 17.6591376 | alive | 17.65914 | no metastasis | 2 |
| 157 | 7.87405886 | alive | 7.874059 | no metastasis | 2 |
| 158 | 3.90691307 | dead | 2.811773 | metastasis | 1 |
| 159 | 5.41546886 | dead | 4.44627 | metastasis | 1 |
| 160 | 16.1478439 | alive | 16.14784 | no metastasis | 2 |
| 161 | 13.4045175 | dead | 8.128679 | metastasis | 2 |
| 162 | 15.3127995 | alive | 15.3128 | no metastasis | 1 |
| 163 | 15.8193019 | alive | 15.8193 | no metastasis | 1 |
| 164 | 5.66461328 | alive | 5.664613 | no metastasis | 1 |
| 165 | 11.0171116 | dead | 10.44216 | metastasis | 1 |
| 166 | 3.62217659 | dead | 1.612594 | metastasis | 1 |
| 167 | 15.3237509 | alive | 15.32375 | no metastasis | 2 |
| 169 | 14.8856947 | alive | 14.88569 | no metastasis | 1 |
| 170 | 13.3497604 | alive | 13.34976 | no metastasis | 2 |
| 172 | 1.63449692 | dead | 1.38809 | metastasis | 1 |
| 174 | 13.7494867 | alive | 13.74949 | no metastasis | 1 |
| 175 | 7.67419576 | dead | 7.594798 | metastasis | 1 |
| 176 | 12.5722108 | alive | 12.57221 | no metastasis | 2 |
| 177 | 9.71115674 | dead | 8.925394 | metastasis | 1 |
| 178 | 13.174538 | alive | 13.17454 | no metastasis | 2 |
| 179 | 12.7638604 | alive | 12.76386 | no metastasis | 1 |
| 180 | 5.28678987 | dead | 2.614648 | metastasis | 1 |
| 181 | 11.8001369 | alive | 11.80014 | no metastasis | 1 |
| 182 | 11.3182752 | alive | 11.31828 | no metastasis | 2 |
| 183 | 11.8603696 | alive | 11.86037 | no metastasis | 2 |
| 184 | 4.40520192 | dead | 1.21013 | metastasis | 1 |
| 185 | 7.33470226 | dead | 7.334702 | no metastasis | 2 |
| 186 | 11.7399042 | dead | 11.7399 | no metastasis | 1 |
| 187 | 12.5037645 | alive | 12.50376 | no metastasis | 2 |
| 188 | 11.2635181 | alive | 11.26352 | no metastasis | 2 |
| 189 | 12.073922 | alive | 12.07392 | no metastasis | 1 |
| 190 | 11.9233402 | alive | 11.92334 | no metastasis | 2 |
| 191 | 12.7364819 | alive | 12.73648 | no metastasis | 2 |
| 192 | 6.29705681 | dead | 2.696783 | metastasis | 1 |
| 193 | 11.8329911 | alive | 11.83299 | no metastasis | 2 |
| 194 | 13.0677618 | alive | 12.46543 | metastasis | 2 |
| 195 | 11.5455168 | alive | 11.54552 | no metastasis | 1 |
| 196 | 11.1950719 | alive | 11.19507 | no metastasis | 2 |
| 197 | 11.0472279 | alive | 11.04723 | no metastasis | 2 |
| 198 | 11.1430527 | alive | 11.14305 | no metastasis | 2 |
| 199 | 10.9075975 | alive | 10.9076 | no metastasis | 1 |
| 200 | 10.7679672 | alive | 10.76797 | no metastasis | 2 |
| 201 | 11.2005476 | alive | 11.20055 | no metastasis | 2 |
| 202 | 4.84599589 | dead | 3.378445 | metastasis | 1 |
| 203 | 11.0362765 | alive | 11.03628 | no metastasis | 1 |
| 205 | 10.1382615 | alive | 10.13826 | no metastasis | 1 |
| 207 | 9.65366188 | alive | 9.653662 | no metastasis | 2 |
| 208 | 10.6748802 | alive | 10.67488 | no metastasis | 1 |
| 209 | 11.4414784 | alive | 6.565366 | metastasis | 2 |
| 210 | 11.2032854 | alive | 11.20329 | no metastasis | 1 |
| 212 | 12.1451061 | dead | 12.14511 | no metastasis | 1 |
| 213 | 3.24709103 | dead | 1.97399 | metastasis | 1 |
| 214 | 10.45859 | alive | 7.477071 | metastasis | 2 |
| 215 | 10.3518138 | alive | 10.35181 | no metastasis | 1 |
| 217 | 1.94661191 | dead | 1.716632 | metastasis | 1 |
| 218 | 2.94592745 | dead | 2.340862 | metastasis | 1 |
| 219 | 9.83162218 | alive | 9.831622 | no metastasis | 2 |
| 220 | 10.3271732 | alive | 10.32717 | no metastasis | 2 |
| 221 | 10.3764545 | alive | 10.37645 | no metastasis | 2 |
| 222 | 3.30732375 | dead | 2.253251 | metastasis | 1 |
| 224 | 10.0205339 | alive | 10.02053 | no metastasis | 1 |
| 226 | 8.79123888 | alive | 8.791239 | no metastasis | 1 |
| 227 | 7.21423682 | dead | 3.356605 | metastasis | 1 |
| 228 | 1.43463381 | dead | 1.223819 | metastasis | 1 |
| 229 | 2.85831622 | dead | 1.61807 | metastasis | 2 |
| 230 | 0.71184121 | dead | 0.271047 | metastasis | 1 |
| 231 | 11.156742 | alive | 3.581109 | metastasis | 2 |
| 233 | 14.1218344 | alive | 14.12183 | no metastasis | 2 |
| 235 | 6.51608487 | alive | 6.516085 | no metastasis | 2 |
| 236 | 2.48323066 | alive | 2.483231 | no metastasis | 1 |
| 237 | 1.31690623 | dead | 1.152635 | metastasis | 1 |
| 238 | 2.15195072 | dead | 1.845311 | metastasis | 1 |
| 239 | 8.09308693 | alive | 8.093087 | no metastasis | 2 |
| 240 | 6.97330596 | alive | 4.095825 | metastasis | 1 |
| 241 | 2.13278576 | dead | 2.004107 | metastasis | 1 |
| 243 | 9.98220397 | alive | 9.982204 | no metastasis | 2 |
| 245 | 11.5455168 | alive | 11.54552 | no metastasis | 1 |
| 246 | 11.449692 | alive | 11.44969 | no metastasis | 2 |
| 247 | 5.63723477 | alive | 5.637235 | no metastasis | 1 |
| 248 | 4.93360712 | alive | 4.933607 | no metastasis | 1 |
| 249 | 5.31690623 | alive | 5.316906 | no metastasis | 1 |
| 250 | 11.3648186 | alive | 11.36482 | no metastasis | 2 |
| 251 | 9.40725531 | alive | 9.407255 | no metastasis | 1 |
| 252 | 9.91649555 | alive | 9.122519 | metastasis | 1 |
| 254 | 4.66803559 | dead | 4.588638 | metastasis | 1 |
| 256 | 9.50581793 | alive | 8.988364 | metastasis | 2 |
| 257 | 2.58726899 | dead | 2.297057 | metastasis | 2 |
| 258 | 5.35249829 | dead | 5.117043 | metastasis | 1 |
| 259 | 8.96372348 | alive | 5.516769 | metastasis | 2 |
| 260 | 8.81314168 | alive | 8.303901 | metastasis | 2 |
| 261 | 8.59411362 | alive | 8.594114 | no metastasis | 2 |
| 263 | 4.5284052 | dead | 2.223135 | metastasis | 1 |
| 264 | 7.25256674 | alive | 7.252567 | no metastasis | 2 |
| 265 | 6.78986995 | alive | 6.78987 | no metastasis | 1 |
| 266 | 7.01163587 | alive | 7.011636 | no metastasis | 1 |
| 267 | 6.92950034 | alive | 6.9295 | no metastasis | 1 |
| 268 | 7.08829569 | alive | 7.088296 | no metastasis | 1 |
| 269 | 1.35249829 | dead | 0.936328 | metastasis | 1 |
| 270 | 2.96235455 | dead | 2.962355 | no metastasis | 1 |
| 271 | 7.02258727 | alive | 7.022587 | no metastasis | 2 |
| 272 | 7.25256674 | alive | 7.252567 | no metastasis | 2 |
| 273 | 6.99794661 | alive | 6.997947 | no metastasis | 1 |
| 274 | 5.9247091 | alive | 5.924709 | no metastasis | 2 |
| 275 | 0.05475702 | alive | 0.054757 | no metastasis | 2 |
| 276 | 1.07323751 | dead | 0.648871 | metastasis | 1 |
| 277 | 5.11430527 | alive | 5.114305 | no metastasis | 2 |
| 278 | 5.31143053 | alive | 5.311431 | no metastasis | 2 |
| 280 | 5.29226557 | alive | 5.292266 | no metastasis | 2 |
| 281 | 7.34017796 | alive | 7.340178 | no metastasis | 2 |
| 282 | 5.74401095 | alive | 5.744011 | no metastasis | 2 |
| 283 | 5.32511978 | alive | 5.32512 | no metastasis | 1 |
| 284 | 5.32238193 | dead | 3.915127 | metastasis | 1 |
| 285 | 5.77138946 | alive | 5.771389 | no metastasis | 2 |
| 286 | 4.94455852 | alive | 4.944559 | no metastasis | 1 |
| 287 | 6.06707734 | alive | 6.067077 | no metastasis | 2 |
| 288 | 1.86721424 | dead | 0.353183 | metastasis | 1 |
| 290 | 4.97193703 | alive | 4.971937 | no metastasis | 2 |
| 291 | 11.652293 | alive | 11.65229 | no metastasis | 1 |
| 292 | 8.36687201 | alive | 8.366872 | no metastasis | 2 |
| 293 | 6.31348392 | alive | 6.313484 | no metastasis | 1 |
| 294 | 6.14373717 | alive | 6.143737 | no metastasis | 1 |
| 295 | 5.55509925 | alive | 5.555099 | no metastasis | 2 |
| 296 | 5.08692676 | alive | 5.086927 | no metastasis | 1 |
| 297 | 9.59616701 | alive | 9.596167 | no metastasis | 2 |
| 298 | 9.45653662 | alive | 9.456537 | no metastasis | 2 |
| 300 | 3.78370979 | dead | 2.852841 | metastasis | 1 |
| 301 | 9.33059548 | alive | 9.330595 | no metastasis | 2 |
| 302 | 1.78234086 | alive | 1.782341 | no metastasis | 1 |
| 303 | 9.19370294 | alive | 9.193703 | no metastasis | 2 |
| 304 | 9.67008898 | alive | 6.710472 | metastasis | 2 |
| 305 | 9.54962355 | alive | 9.549624 | no metastasis | 2 |
| 306 | 10.201232 | alive | 10.20123 | no metastasis | 2 |
| 307 | 2.80629706 | dead | 1.965777 | metastasis | 1 |
| 308 | 9.32238193 | alive | 9.322382 | no metastasis | 1 |
| 309 | 9.31416838 | alive | 8.561259 | metastasis | 2 |
| 310 | 9.09787817 | alive | 9.097878 | no metastasis | 1 |
| 311 | 4.54757016 | dead | 4.219028 | metastasis | 1 |
| 312 | 9.10335387 | alive | 9.103354 | no metastasis | 2 |
| 313 | 9.03216975 | alive | 6.056126 | metastasis | 2 |
| 314 | 5.05954826 | dead | 3.219713 | metastasis | 1 |
| 315 | 8.24093087 | alive | 8.240931 | no metastasis | 2 |
| 317 | 5.60438056 | dead | 2.138261 | metastasis | 2 |
| 318 | 2.43394935 | alive | 2.335387 | metastasis | 2 |
| 319 | 6.49965777 | dead | 6.370979 | metastasis | 1 |
| 320 | 9.89459275 | alive | 9.894593 | no metastasis | 1 |
| 321 | 1.78507871 | alive | 1.500342 | metastasis | 2 |
| 322 | 6.70499658 | alive | 6.704997 | no metastasis | 1 |
| 323 | 8.80219028 | alive | 8.80219 | no metastasis | 2 |
| 324 | 8.85968515 | alive | 8.859685 | no metastasis | 1 |
| 325 | 8.85420945 | alive | 8.854209 | no metastasis | 2 |
| 326 | 8.29842574 | alive | 8.298426 | no metastasis | 1 |
| 327 | 6.09445585 | alive | 4.621492 | metastasis | 1 |
| 328 | 5.57700205 | alive | 5.577002 | no metastasis | 2 |
| 329 | 5.80424367 | alive | 5.804244 | no metastasis | 1 |
| 330 | 5.19917865 | alive | 5.199179 | no metastasis | 1 |
| 331 | 2.50787132 | dead | 2.157426 | metastasis | 1 |
| 332 | 7.99178645 | alive | 7.991786 | no metastasis | 1 |
| 333 | 8.49555099 | alive | 8.495551 | no metastasis | 1 |
| 334 | 7.69336071 | alive | 7.693361 | no metastasis | 2 |
| 335 | 7.4770705 | alive | 7.477071 | no metastasis | 1 |
| 336 | 7.40862423 | alive | 7.408624 | no metastasis | 2 |
| 337 | 6.81998631 | alive | 6.819986 | no metastasis | 1 |
| 338 | 6.34360027 | alive | 6.3436 | no metastasis | 1 |
| 339 | 16.5913758 | alive | 16.59138 | no metastasis | 1 |
| 340 | 5.85900068 | dead | 3.12115 | metastasis | 1 |
| 341 | 2.36276523 | dead | 1.73306 | metastasis | 1 |
| 342 | 15.3511294 | alive | 15.35113 | no metastasis | 2 |
| 343 | 6.6091718 | alive | 6.609172 | no metastasis | 2 |
| 344 | 6.87474333 | alive | 6.874743 | no metastasis | 1 |
| 345 | 6.99520876 | alive | 6.995209 | no metastasis | 2 |
| 346 | 7.1211499 | alive | 7.12115 | no metastasis | 2 |
| 347 | 4.72005476 | alive | 4.720055 | no metastasis | 2 |
| 348 | 6.17111567 | alive | 6.171116 | no metastasis | 2 |
| 349 | 6.46406571 | alive | 6.464066 | no metastasis | 1 |
| 350 | 3.28542095 | alive | 3.285421 | no metastasis | 1 |
| 351 | 6.52703628 | alive | 6.527036 | no metastasis | 1 |
| 352 | 5.80971937 | alive | 5.809719 | no metastasis | 2 |
| 353 | 6.55167693 | alive | 6.551677 | no metastasis | 1 |
| 354 | 6.16016427 | alive | 6.160164 | no metastasis | 2 |
| 355 | 6.04517454 | alive | 6.045175 | no metastasis | 2 |
| 356 | 6.21492129 | alive | 6.214921 | no metastasis | 2 |
| 357 | 5.82340862 | alive | 5.823409 | no metastasis | 2 |
| 358 | 6.23956194 | alive | 6.239562 | no metastasis | 2 |
| 359 | 6.01779603 | alive | 6.017796 | no metastasis | 2 |
| 360 | 5.54962355 | alive | 5.549624 | no metastasis | 2 |
| 361 | 5.34702259 | alive | 5.347023 | no metastasis | 2 |
| 362 | 5.25941136 | alive | 5.259411 | no metastasis | 1 |
| 363 | 6.00958248 | alive | 4.971937 | metastasis | 1 |
| 364 | 18.0807666 | alive | 18.08077 | no metastasis | 1 |
| 365 | 17.486653 | alive | 17.48665 | no metastasis | 2 |
| 366 | 17.1526352 | alive | 17.15264 | no metastasis | 2 |
| 367 | 0.97467488 | dead | 0.572211 | metastasis | 1 |
| 368 | 16.8706366 | alive | 9.568789 | metastasis | 2 |
| 369 | 6.57084189 | dead | 3.258042 | metastasis | 2 |
| 370 | 14.3600274 | dead | 9.998631 | metastasis | 2 |
| 371 | 2.40657084 | dead | 1.968515 | metastasis | 2 |
| 373 | 7.77275839 | alive | 7.772758 | no metastasis | 2 |
| 374 | 5.75496236 | dead | 2.680356 | metastasis | 1 |
| 375 | 17.4209446 | alive | 17.42094 | no metastasis | 1 |
| 377 | 9.53045859 | dead | 8.528405 | metastasis | 1 |
| 378 | 13.9192334 | alive | 13.91923 | no metastasis | 1 |
| 379 | 13.8644764 | alive | 13.86448 | no metastasis | 1 |
| 380 | 12.7392197 | alive | 12.73922 | no metastasis | 2 |
| 381 | 12.2600958 | alive | 12.2601 | no metastasis | 2 |
| 383 | 11.08282 | alive | 11.08282 | no metastasis | 2 |
| 385 | 2.88843258 | dead | 1.946612 | metastasis | 1 |
| 387 | 8.21355236 | alive | 8.213552 | no metastasis | 2 |
| 388 | 7.22518823 | alive | 7.225188 | no metastasis | 2 |
| 389 | 4.94729637 | dead | 3.419576 | metastasis | 1 |
| 390 | 6.80355921 | alive | 6.803559 | no metastasis | 2 |
| 391 | 6.02053388 | alive | 6.020534 | no metastasis | 2 |
| 392 | 6.17111567 | alive | 6.171116 | no metastasis | 1 |
| 393 | 5.5742642 | alive | 5.574264 | no metastasis | 1 |
| 394 | 5.70841889 | alive | 5.708419 | no metastasis | 2 |
| 395 | 15.0773443 | alive | 11.2115 | metastasis | 2 |
| 396 | 10.2313484 | alive | 10.23135 | no metastasis | 1 |
| 397 | 8.77207392 | dead | 4.766598 | metastasis | 2 |
| 398 | 8.42436687 | alive | 8.424367 | no metastasis | 1 |
| 401 | 10.0314853 | alive | 1.527721 | metastasis | 1 |
| 402 | 7.37850787 | alive | 7.378508 | no metastasis | 1 |
| 403 | 6.75427789 | alive | 6.754278 | no metastasis | 2 |
| 404 | 7.57015743 | alive | 7.570157 | no metastasis | 2 |

### REFERENCES

1. Gupta, G. P. & Massague, J. Cancer metastasis: building a framework. Cell 127, 679-95 (2006).
2. Bernards, R. & Weinberg, R. A. A progression puzzle. Nature 418, 823 (2002).
3. Perou, C. M. et al. Molecular portraits of human breast tumours. Nature 406, 747-52 (2000).
4. Ramaswamy, S., Ross, K. N., Lander, E. S. & Golub, T. R. A molecular signature of metastasis in primary solid tumors. Nat Genet 33, 49-54 (2003).
5. van't Veer, L. J. et al. Gene expression profiling predicts clinical outcome of breast cancer. Nature 415, 530-6 (2002).
6. van de Vijver, M. J. et al. A gene-expression signature as a predictor of survival in breast cancer. N Engl J Med 347, 1999-2009 (2002).
7. Chin, K. et al. In situ analyses of genome instability in breast cancer. Nat Genet 36, 984-8 (2004).
8. Rudolph, K. L., Millard, M., Bosenberg, M. W. & DePinho, R. A. Telomere dysfunction and evolution of intestinal carcinoma in mice and humans. Nat Genet 28, 155-9 (2001).
9. Breslow, A. Thickness, cross-sectional areas and depth of invasion in the prognosis of cutaneous melanoma. Ann Surg 172, 902-8 (1970).
10. Gimotty, P. A. et al. Biologic and prognostic significance of dermal Ki67 expression, mitoses, and tumorigenicity in thin invasive cutaneous melanoma. J Clin Oncol 23, 8048-56 (2005).
11. Gimotty, P. A. et al. Identification of high-risk patients among those diagnosed with thin cutaneous melanomas. J Clin Oncol 25, 1129-34 (2007).
12. Chin, L. et al. Cooperative effects of INK4a and ras in melanoma susceptibility in vivo. Genes and Development 11, 2822-34 (1997).
13. Natali, P. G. et al. Expression of the c-Met/HGF receptor in human melanocytic neoplasms: demonstration of the relationship to malignant melanoma tumour progression. Br J Cancer 68, 746-50 (1993).
14. Chin, L. et al. Essential role for oncogenic Ras in tumour maintenance. Nature 400, 468-72 (1999).
15. Garraway, L. A. et al. Integrative genomic analyses identify MITF as a lineage survival oncogene amplified in malignant melanoma. Nature 436, 117-22 (2005).
16. Kim, M. et al. Comparative oncogenomics identifies NEDD9 as a melanoma metastasis gene. Cell 125, 1269-81 (2006).
17. Chariot, A. & Castronovo, V. Detection of HOXA1 expression in human breast cancer. Biochem Biophys Res Commun 222, 292-7 (1996).
18. Maeda, K. et al. Altered expressions of HOX genes in human cutaneous malignant melanoma. Int J Cancer 114, 436-41 (2005).
19. Abe, M. et al. Disordered expression of HOX genes in human non-small cell lung cancer. Oncol Rep 15, 797-802 (2006).
20. Schlaepfer, D. D. & Mitra, S. K. Multiple connections link FAK to cell motility and invasion. Curr Opin Genet Dev 14, 92-101 (2004).
21. Bierie, B. & Moses, H. L. Tumour microenvironment: TGFbeta: the molecular Jekyll and Hyde of cancer. Nat Rev Cancer 6, 506-20 (2006).
22. Weinberg, R. A. Mechanisms of Malignant Progression. Carcinogenesis (2008).
23. Winnepenninckx, V. et al. Gene expression profiling of primary cutaneous melanoma and clinical outcome. J Natl Cancer Inst 98, 472-82 (2006).
24. Rhodes, D. R. et al. ONCOMINE: a cancer microarray database and integrated data-mining platform. Neoplasia 6, 1-6 (2004).
25. Rhodes, D. R. & Chinnaiyan, A. M. Integrative analysis of the cancer transcriptome. Nat Genet 37 Suppl, S31-7 (2005).
26. Ren, B. et al. MCM7 amplification and overexpression are associated with prostate cancer progression. Oncogene 25, 1090-8 (2006).

We claim:

1. A method with a predetermined level of predictability for assessing a risk of development of a metastatic tumor in a subject comprising:
a. measuring the level of two or more DETERMINANTS selected from the group consisting of DETERMINANTS 1-25, 41, 61, 62, 63, 66, 74, 96, 99, 103, 126, 135, 137, 138, 177, 190, 210, 212, 217, 218, 227, 239, 261, and 271 in a sample from the subject, and
b. measuring a clinically significant alteration in the level of the two or more DETERMINANTS in the sample, wherein the alteration indicates an increased risk of developing a metastatic tumor in the subject.

2. The method of claim 1, further comprising measuring an effective amount of one or more DETERMINANTS selected from the group consisting of
DETERMINANTS 26-40, 42-60, 64, 65, 67-73, 75-95, 97, 98, 100-102, 104-125, 127-134, 136, 139-176, 178-189, 191-209, 211, 213-216, 219-226, 228-238, 240-260, 262-270, 272-360.

3. The method of claim 1 or 2, further comprising measuring at least one standard parameters associated with said tumor.

4. The method of claim 1, wherein the level of a DETERMINANT is measured electrophoretically or immunochemically.

5. The method of claim 4, wherein the immunochemical detection is by radioimmunoassay, immunofluorescence assay or by an enzyme-linked immunosorbent assay.

6. The method of claim 1, wherein the subject has a primary tumor, a recurrent tumor, or a metastatic tumor.

7. The method of claim 1, wherein the sample is a tumor biopsy.

8. The method of claim 1, wherein said biopsy is a core biopsy, an excisional tissue biopsy or an incisional tissue biopsy.

9. The method of claim 1, wherein the level of expression of five or more DETERMINANTS is measured.

10. A method with a predetermined level of predictability for assessing for assessing a risk of development of a metastatic tumor in a subject comprising:
a. measuring the level of two or more DETERMINANTS selected from the group consisting of DETERMINANTS 1-25, 41, 61, 62, 63, 66, 74, 96, 99, 103, 126, 135, 137, 138, 177, 190, 210, 212, 217, 218, 227, 239, 261, and 271 in a sample from the subject, and
b. comparing the level of the two or more DETERMINANTS to a reference value.

11. The method of claim 10, wherein the reference value is an index value.

12. A method with a predetermined level of predictability for assessing the progression of a tumor in a subject comprising:
a. detecting the level of two or more DETERMINANTS selected from the group consisting of DETERMINANTS 1-25, 41, 61, 62, 63, 66, 74, 96, 99, 103, 126, 135, 137, 138, 177, 190, 210, 212, 217, 218, 227, 239, 261, and 271 in a first sample from the subject at a first period of time;
b. detecting the level of two or more DETERMINANTS in a second sample from the subject at a second period of time;
c. comparing the level of the two or more DETERMINANTS detected in step (a) to the amount detected in step (b), or to a reference value.

13. The method of claim 12, wherein the first sample is taken from the subject prior to being treated for the tumor.

14. The method of claim 2, wherein the second sample is taken from the subject after being treated for the tumor.

15. A method with a predetermined level of predictability for monitoring the effectiveness of treatment for a metastatic tumor:
a. detecting the level of two or more DETERMINANTS selected from the group consisting of DETERMINANTS 1-25, 41, 61, 62, 63, 66, 74, 96, 99, 103, 126, 135, 137, 138, 177, 190, 210, 212, 217, 218, 227, 239, 261, and 271 in a first sample from the subject at a first period of time;
b. detecting the level of two or more DETERMINANTS in a second sample from the subject at a second period of time;
c. comparing the level of the two or more DETERMINANTS detected in step (a) to the amount detected in step (b), or to a reference value, wherein the effectiveness of treatment is monitored by a change in the level of two or more DETERMINANTS from the subject.

16. The method of claim 15, wherein the subject has previously been treated for the metastatic tumor.

17. The method of claim 15, wherein the first sample is taken from the subject prior to being treated for the metastatic tumor.

18. The method of claim 15, wherein the second sample is taken from the subject after being treated for the metastatic tumor.

19. A method with a predetermined level of predictability for selecting a treatment regimen for a subject diagnosed a tumor comprising:
a. detecting the level of an effective amount of two or more DETERMINANTS selected from the group consisting of DETERMINANTS 1-25, 41, 61, 62, 63, 66, 74, 96, 99, 103, 126, 135, 137, 138, 177, 190, 210, 212, 217, 218, 227, 239, 261, and 271 in a first sample from the subject at a first period of time;
b. optionally detecting the level of an effective amount of two or more DETERMINANTS in a second sample from the subject at a second period of time;
c. comparing the level of the two or more DETERMINANTS detected in step (a) to a reference value, or optionally, to the amount detected in step (b).

20. The method of claim 19, wherein the subject has previously been treated for the tumor.

21. The method of claim 19, wherein the first sample is taken from the subject prior to being treated for the tumor.

22. The method of claim 19, wherein the second sample is taken from the subject after being treated for the tumor.

23. A metastatic tumor reference expression profile, comprising a pattern of marker levels of an effective amount of two or more markers selected from the group consisting of DETERMINANTS 1-25, 41, 61, 62, 63, 66, 74, 96, 99, 103, 126, 135, 137, 138, 177, 190, 210, 212, 217, 218, 227, 239, 261, and 271

24. A kit comprising a plurality of DETERMINANT detection reagents that detect the corresponding DETERMINANTS selected from the group consisting of DETERMINANTS 1-25, 41, 61, 62, 63, 66, 74, 96, 99, 103, 126, 135, 137, 138, 177, 190, 210, 212, 217, 218, 227, 239, 261, and 271, sufficient to generate the profile of claim 20.

25. The kit of claim 24, wherein the detection reagent comprises one or more antibodies or fragments thereof.

26. The kit of claim 24, wherein the detection reagent comprises one or more oligonucleotides.

27. The kit of claim 24, wherein the detection reagent comprises one or more aptamers.

28. A machine readable media containing one or more metastatic tumor reference expression profiles according to claim 23, and optionally, additional test results and subject information.

29. A DETERMINANT panel comprising one or more DETERMINANTS that are indicative of a physiological or biochemical pathway associated metastasis.

30. The panel of claim 26, wherein the physiological or biochemical pathway comprises cell migration, angiogenesis, extracellular matrix degradation or anoikis.

31. A DETERMINANT panel comprising one or more DETERMINANTS that are indicative of the progression of a tumor.

32. A method of identifying a biomarker that is prognostic for a disease comprising:
a) identifying one or more genes that are differentially expressed in said disease compared to a control to produce a gene target list; and
b) identifying one or more genes on said target list that is associated with a functional aspect of the progression of said disease.
thereby identifying a biomarker that is prognostic for said disease.

33. The method of claim 32, further comprising the step of identifying one or more genes on said gene target list that comprise an evolutionarily conserved change to produce a second gene target list.

34. The method of claim 32, wherein said disease is cancer.

35. The method of claim 34, wherein said cancer is metastatic cancer.

36. The method of claim 32, wherein said functional aspect is cell migration, angiogenesis, extracellular matrix degradation or anoikis.

37. A method of identifying a compound that modulates the activity or expression of a DETERMINANT comprising
(a) providing a cell expressing the DETERMINANT;
(b) contacting the cell with a composition comprising a candidate compound; and
(c) determining whether the substance alters the expression or activity of the DETERMINANT;
whereby, if the alteration observed in the presence of the compound is not observed when the cell is contacted with a composition devoid of the compound, the compound identified modulates the activity or expression of a DETERMINANT.

38. The method of claim 37 wherein said cell is contacted in vivo, ex vivo or in vitro.

39. A method of treating a cancer in a subject comprising administering to said subject a compound that modulates the activity or expression of a DETERMINANT.

40. A method of treating a cancer in a subject comprising administering to said subject an agent that modulates the activity or expression of a compound that is modulated by a DETERMINANT.

41. The method of claim 40, wherein said compound is TGFβ or CXCR4

42. The method of claim 41, wherein said agent is a TGFβ inhibitor or a CXCR4 inhibitor.

43. A method of treating a patient with a tumor, comprising:
identifying a patient with a tumor, wherein two or more of DETERMINANTS 1-360 are altered in a clinically significant manner as measured in a sample from the tumor, and
treating the patient with a therapeutic regimen that prevents or reduces tumor metastasis.

44. A method of selecting a tumor patient in need of adjuvant treatment, comprising:
assessing the risk of metastasis in the patient by measuring two or more of DETERMINANTS 1-360, wherein clinically significant alteration of said two or more DETERMINANTS in a tumor sample from the patient indicates that the patient is in need of adjuvant treatment.

45. A method of informing a treatment decision for a tumor patient, comprising:
obtaining information on two or more of DETERMINANTS 1-360 in a tumor sample from the patient, and
selecting a treatment regimen that prevents or reduces tumor metastasis in the patient if said two or more DETERMINANTS are altered in a clinically significant manner.


SEQUENCE LISTING (12 sequences)

SEQ ID NO: 1 - DNA, 30 bp, Artificial Sequence
  Primer sequence
tctgttgccatcccaagacaacattgatgg

SEQ ID NO: 2 - DNA, 20 bp, Artificial Sequence
  Primer sequence
aaatctctggaggaggttgg

SEQ ID NO: 3 - DNA, 20 bp, Artificial Sequence
  Primer sequence
caaggccaaggagaaggtta

SEQ ID NO: 4 - DNA, 20 bp, Artificial Sequence
  Primer sequence
tttgaagttctcgggagtga

SEQ ID NO: 5 - DNA, 20 bp, Artificial Sequence
  Primer sequence
ccagaagccaatgcacctat

SEQ ID NO: 6 - DNA, 20 bp, Artificial Sequence
  Primer sequence
agcaataacagctcccacca

SEQ ID NO: 7 - DNA, 20 bp, Artificial Sequence
  Primer sequence
attctggcctccagttacca

SEQ ID NO: 8 - DNA, 20 bp, Artificial Sequence
  Primer sequence
ggcttcattcttggtgcttc

SEQ ID NO: 9 - DNA, 20 bp, Artificial Sequence
  Primer sequence
aacaacccttccacagatgc

SEQ ID NO: 10 - DNA, 20 bp, Artificial Sequence
  Primer sequence
tctccattaagggcgcatag

SEQ ID NO: 11 - DNA, 20 bp, Artificial Sequence
  Primer sequence
cttccgcaagttcacctacc

SEQ ID NO: 12 - DNA, 20 bp, Artificial Sequence
  Primer sequence
tacttgagggggatgaatcg